SEQUENCE LISTING (3 sequences)

SEQ ID NO: 1 - skipped

SEQ ID NO: 2 - protein, 14 aa, synthetic construct
KDDYAGGGAAGKDA

SEQ ID NO: 3 - skipped

(19) United States
(12) Patent Application Publication
(10) Pub. No.: US 2024/0426831 A1
(43) Pub. Date: Dec. 26, 2024

(54) STRUCTURAL PROFILING OF NATIVE PROTEINS USING FLUOROSEQUENCING, A SINGLE MOLECULE PROTEIN SEQUENCING TECHNOLOGY

(71) Applicants: BOARD OF REGENTS, THE UNIVERSITY OF TEXAS SYSTEM, Austin, TX (US); ERISYON, INC., Austin, TX (US)

(72) Inventors: Julia BROWN, Austin, TX (US); Emily BABCOCK, Austin, TX (US); Jagannath SWAMINATHAN, Austin, TX (US)

(21) Appl. No.: 18/697,605

(22) PCT Filed: Sep. 30, 2022
(86) PCT No.: PCT/US2022/077340

§ 371 Date: Apr. 1, 2024

Related U.S. Application Data
(60) Provisional application No. 63/251,512, filed on Oct. 1, 2021.

(51) Int. Cl. G01N 33/68; G01N 33/543; G01N 33/573

(52) U.S. Cl. CPC G01N 33/6824; G01N 33/54386; G01N 33/573; G01N 33/6848; G01N 33/6896

(57) ABSTRACT

The present disclosure provides methods, systems, and/or kits for analyzing a polypeptide. The method and systems may comprise a native protein coupled to a support, wherein the native protein comprises one or more internal amino acids and one or more external amino acids, and wherein the native protein comprises one or more labels coupled to said one or more external amino acids.

### CROSS-REFERENCE

This application claims the benefit of U.S. Application No. 63/251,512, filed Oct. 1, 2021, which is incorporated by reference herein in its entirety.

### BACKGROUND

Protein aggregation is a common characteristic of multiple diseases (e.g., neurodegenerative diseases). An abundance of misfolded proteins leading to aggregates and/or oligomers appears to be toxic to cells, leading to cell damage and eventually cell death. For example, accumulation of amyloid-forming misfolded proteins may lead to a wide range of diseases such as amyloidoses. Similarly, Alzheimer's disease (AD) neuropathology is characterized by accumulation of misfolded amyloid beta protein and/or neurofibrillary tangles comprising tau in the central nervous system, synaptic loss, and neuronal death. In these diseases, it can be difficult to detect the presence of misfolded proteins based on protein sequence information alone.

### SUMMARY

The present disclosure provides methods, systems, and/or kits for analyzing a protein and methods, systems, and/or kits for determining the structure of a protein.

In some aspect, the present disclosure provides a method, comprising: providing a native protein coupled to a support, wherein the native protein comprises a native structure, wherein the native protein comprises one or more internal amino acids and one or more external amino acids, wherein the native protein comprises one or more labels coupled to the one or more external amino acids.

In some embodiments, the method further comprises detecting the one or more labels coupled to the one or more external amino acids to determine the native structure of the native protein using the detected one or more labels. In some embodiments, the detecting comprises detecting a sequence pattern of the one or more labels coupled to the one or more external amino acids. In some embodiments, determining the native structure of the native protein comprises identifying a misfolded protein.

In some embodiments, the one or more external amino acids comprises a surface-exposed amino acid residue.

In some embodiments, the method further comprises coupling said one or more labels to said one or more external amino acids. In some embodiments, the method further comprises coupling the native protein to the support prior to coupling the one or more labels to the one or more external amino acids.

In some embodiments, the method further comprises detecting the one or more labels coupled to the one or more external amino acids to quantify the native protein.

In some embodiments, the method further comprises releasing the native protein from the support. In some embodiments, the method further comprises digesting the native protein into one or more peptides. In some embodiments, wherein each of the one or more peptides has from about 5 amino acids to about 50 amino acids. In some embodiments, the method further comprises coupling one or more additional labels to the one or more internal amino acids. In some embodiments, the method further comprises detecting the one or more labels coupled to the one or more external amino acids. In some embodiments, the method further comprises coupling the one or more labels to the one or more external amino acids. In some embodiments, the method further comprises detecting one or more additional labels coupled to the one or more internal amino acids.

In some embodiments, the method further comprises using the one or more labels and one or more additional labels to determine the native structure of the native protein.

In some embodiments, the native structure of the native protein comprises a tertiary structure of the native protein.

In some embodiments, the one or more labels comprise an optical label. In some embodiments, the optical label comprises a fluorescent dye.

In some embodiments, the method further comprises detecting the native structure of the native protein while the native protein is coupled to the support.

In some embodiments, the native protein comprising the native structure is immobilized to the support.

In some embodiments, the native protein is covalently-coupled to the support.

In some embodiments, the native protein is reversibly-coupled to said support.

In some embodiments, a surface of the support comprises one or more maleic anhydride groups.

In some embodiments, the one or more labels comprise an amino acid type-specific label. In some embodiments, the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

In one aspect, the present disclosure provides a system comprising: a native protein coupled to a support, wherein the native protein comprises a native structure, wherein the native protein comprises one or more internal amino acids and one or more external amino acids, wherein the native protein comprises one or more labels coupled to the one or more external amino acids.

In some embodiments, the native structure comprises a tertiary structure of the native protein.

In some embodiments, the one or more labels are covalently-coupled to the one or more external amino acids.

In some embodiments, the one or more labels comprise an optical label. In some embodiments, the optical label comprises a fluorescent dye.

In some embodiments, the native protein is covalently-coupled to the support.

In some embodiments, the native protein is reversibly-coupled to the support.

In some embodiments, a surface of the support comprises one or more maleic anhydride groups.

In some embodiments, the one or more labels comprise an amino acid type-specific label. In some embodiments, amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

In some embodiments, the one or more external amino acids comprises a surface-exposed amino acid residue.

In some embodiments, the system further comprises a detector configured to detect the one or more labels. In some embodiments, the detector comprises an intensified charge-couple device (CCD) detector or a complementary metal-oxide semiconductor (CMOS) detector. In some embodiments, the system further comprises a computer processor communicatively coupled to the detector, wherein the computer processor is programmed to detect one or more signals from the detector. In some embodiments, the one or more signals are from the one or more labels coupled to the one or more external amino acids. In some embodiments, the computer processor is programmed to determine the native structure of the native protein using the one or more labels detected by the detector. In some embodiments, the computer processor is programmed to detect fluorescence signals. In some embodiments, the computer processor is programmed to distinguish each of the one or more signals from the detector. In some embodiments, the computer processor is programmed to quantify the one or more signals from the detector.

One aspect of the present disclosure provides a method, comprising: providing a native protein, wherein the native protein comprises a native structure, wherein the native protein comprises one or more internal amino acids and one or more external amino acids, wherein the native protein comprises one or more optically detectable labels coupled to the one or more external amino acids.

In some embodiments, the method further comprises detecting the one or more optically detectable labels coupled to the one or more external amino acids to identify the native structure of the native protein using the detected one or more optically detectable labels. In some embodiments, the detecting comprises detecting a sequence pattern of the one or more labels coupled to the one or more external amino acids. In some embodiments, determining the native structure of the native protein comprises identifying a misfolded protein.

In some embodiments, the one or more external amino acids comprises a surface-exposed amino acid residue.

In some embodiments, the method further comprises coupling the one or more labels to the one or more external amino acids. In some embodiments, the method further comprises coupling the native protein to the support prior to coupling the one or more labels to the one or more external amino acids.

In some embodiments, the method further comprises detecting the one or more labels coupled to the one or more external amino acids to quantify the native protein.

In some embodiments, the method further comprises releasing the native protein from the support. In some embodiments, the method further comprises digesting the native protein into one or more peptides. In some embodiments, each of the one or more peptides has from about 5 amino acids to about 50 amino acids. In some embodiments, the method further comprises coupling one or more additional labels to the one or more internal amino acids. In some embodiments, the method further comprises detecting the one or more labels coupled to the one or more external amino acids. In some embodiments, the method further comprises coupling the one or more labels to the one or more external amino acids. In some embodiments, the method further comprises detecting one or more additional labels coupled to the one or more internal amino acids.

In some embodiments, the method further comprises using the one or more labels and one or more additional labels to determine the native structure of the native protein.

In some embodiments, the native structure of said native protein comprises a tertiary structure of the native protein.

In some embodiments, the one or more labels comprise an optical label. In some embodiments, the optical label comprises a fluorescent dye.

In some embodiments, the method further comprises detecting the native structure of the native protein while the native protein is coupled to the support.

In some embodiments, the native protein comprising the native structure is immobilized to the support.

In some embodiments, the native protein is covalently-coupled to the support.

In some embodiments, the native protein is reversibly-coupled to the support.

In some embodiments, a surface of the support comprises one or more maleic anhydride groups.

In some embodiments, the one or more labels comprise an amino acid type-specific label. In some embodiments, the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

One aspect of the present disclosure provides a system, comprising: a native protein, wherein the native protein comprises a native structure, wherein the native protein comprises one or more internal amino acids and one or more external amino acids, wherein the native protein comprises one or more optically detectable labels coupled to one or more external amino acids.

In some embodiments, the native structure comprises a tertiary structure of the native protein.

In some embodiments, the one or more labels are covalently-coupled to the one or more external amino acids.

In some embodiments, the one or more labels comprise an optical label. In some embodiments, the optical label comprises a fluorescent dye.

In some embodiments, the native protein is covalently-coupled to the support.

In some embodiments, the native protein is reversibly-coupled to the support.

In some embodiments, a surface of the support comprises one or more maleic anhydride groups.

In some embodiments, the one or more labels comprise an amino acid type-specific label. In some embodiments, the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

In some embodiments, the one or more external amino acids comprises a surface-exposed amino acid residue.

In some embodiments, the system further comprises a detector configured to detect the one or more optically detectable labels. In some embodiments, the detector comprises an intensified charge-couple device (CCD) detector or a complementary metal-oxide semiconductor (CMOS) detector. In some embodiments, the system further comprises a computer processor communicatively coupled to the detector, wherein the computer processor is programmed to detect one or more signals from the detector. In some embodiments, the one or more signals are from the one or more optically detectable labels detected by the detector. In some embodiments, the computer processor is programmed to determine the native structure of the native protein using the one or more optically detectable labels detected by the detector. In some embodiments, the computer processor is programmed to detect fluorescence signals. In some embodiments, the computer processor is programmed to distinguish each of the one or more signals from the detector. In some embodiments, the computer processor is programmed to quantify the one or more signals from the detector.

Another aspect of the present disclosure provides a non-transitory computer readable medium comprising machine executable code that, upon execution by one or more computer processors, implements any of the methods above or elsewhere herein.

Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement methods provided herein.

FIG. 2 schematically illustrates an example of a method for capturing, labeling, and/or detecting a protein and/or peptide;

FIG. 3 illustrates an example of a method for capturing and/or releasing a protein and/or peptide; and

FIG. 4 schematically illustrates an example of a method of capturing, labeling, and/or detecting a protein structure.

### DETAILED DESCRIPTION

Protein aggregation is a common characteristic of multiple diseases (e.g., neurodegenerative diseases). An abundance of misfolded proteins leading to aggregates and/or oligomers appears to be toxic to cells, leading to cell damage and eventually cell death. For example, accumulation of amyloid-forming misfolded proteins may lead to a wide range of diseases such as amyloidoses. Similarly, Alzheimer's disease (AD) neuropathology is characterized by accumulation of misfolded amyloid beta protein and/or neurofibrillary tangles comprising tau in the central nervous system, synaptic loss, and neuronal death. In these diseases, it can be difficult to detect the presence of misfolded proteins based on protein sequence information alone.

Methods for determining structural conformers of proteins range from generating atomic resolution structures (e.g., cryo-Electron microscopy, x-ray crystallography, and/or nuclear magnetic resonance (NMR) spectroscopy) to predictions using low resolution methods (e.g., circular dichroism, Fourier-transform infrared spectroscopy (FTIR), and/or solvent accessibility). These methods are ideally suited for generating and understanding protein structures produced through recombinant approaches and analyzed at a population level (e.g., in bulk). While newer methods using cross-linking and/or covalent protein painting are being developed to determine structural changes in native proteomes, they are bound by the detection limits of mass-spectrometry and the associated challenges in assessing significant proteoforms in low abundance. Thus, a method for profiling the structural heterogeneity of proteins present at low abundances and/or in their native biological milieu is required.

Provided herein are methods, systems, and kits for analyzing (e.g., detecting presence of a biological molecule, quantifying a native protein, non-native protein, non-native polypeptide, and/or non-native peptide, determining the structure of a biological molecule, detecting presence of a misfolded protein) a biological molecule (e.g., a protein, a biological aggregate, a polypeptide, or a polypeptide complex). Also provided herein are methods, systems, and kits for detecting a disease or disorder by analyzing a biological molecule (e.g., a protein, a biological aggregate, a polypeptide, or a polypeptide complex).

The effect of mutations observed in DNA (e.g., single nucleotide polymorphisms (SNP), frame-shifts) and heterogeneity observed in the RNA transcripts (e.g., transcriptional errors and/or splicing events) on translated proteins and their structure may not be known. While some types of mutations can have minimal effect on the protein's structure, others may be cytotoxic and/or cause protein aggregation. Complicating this correlation is the fact that proteoforms that may have a higher tendency to aggregate with native forms, leading to disease progressions (e.g., Alzheimer's Disease and/or Parkinson's Disease) are often in low abundance. Thus, along with detecting mutated genomes and/or low level altered transcriptomes, quantifying the low abundance proteoforms and/or characterizing their structure may best represent the functional state of protein in a sample.

Improvements in diagnostic techniques, such as, for example, assays for detecting misfolded proteins and/or aggregates of misfolded proteins may advance methods for treating and/or managing diseases or disorders. As recognized herein, improved detection methods may be used for detecting protein aggregates and/or misfolded proteins that may be toxic to cells and/or that may cause diseases such as neurodegenerative diseases. Methods to accurately detect and/or quantify misfolded protein structures and/or quantify the number of misfolded protein exist within a protein structure may be used to diagnose, identify or determine the disease stage, and/or find or optimize a treatment for these disease(s).

For example, changes in protein folding may lead to misfunctioning and/or nonfunctional proteins. These misfunctioning and/or nonfunctional proteins can lead to development of a protein confirmational diseases or disorders, such as, for example, Alzheimer's, to protein accumulation or aggregates in form of amorphous, oligomers, amyloid fibrils, etc. The extent (e.g., quantity, type, and/or quality) of protein aggregation may be correlated with progression or state of a protein conformational diseases or disorders, such as, for example, in prion diseases (e.g., Taupathies, synucleinopathies, etc). Therefore, techniques that may identify or determine the presence or absence of proteins formations and/or misfolded proteins and/or precisely measure the extent of protein misfolding (e.g., number of monomer units in an oligomer, quantify number of misfolded proteins) may be instrumental in diagnosing and/or treating such diseases. These techniques may help diagnose, identify or determine the stage of the disease, track progression of the disease, measure effectiveness of various treatments, or optimize treatment regiments. These techniques can also be applied to determine of unknown protein structures. These techniques can be applied to determine at least an unknown portion of a protein structure. These techniques may be used to determine the structure of an unknown protein from a sample from a subject, without the additional processing (e.g., protein crystallization, protein solubilization, production of diffraction pattern, incubation with isotopic buffers) and/or analytical operations (e.g., analysis of diffraction pattern, analysis of peaks of N-NMR spectroscopy) required in other protein structure assay (e.g., X-ray crystallography, nuclear magnetic resonance (NMR)).

While various embodiments of the methods, systems, and/or kits have been shown and described herein, it will be obvious to those skilled in the art that such embodiments are provided by way of example only. Numerous variations, changes, and substitutions may occur to those skilled in the art without departing from the methods, systems, and/or kits. It may be understood that various alternatives to the embodiments of the methods, systems, and/or kits described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “analyte” or “analytes,” as used herein, generally refers to a molecule whose presence or absence may be measured or identified. An analyte can be a molecule for which a detectable probe or assay exists or can be produced. For example, an analyte can be a macromolecule, such as, for example, a nucleic acid, a polypeptide, a carbohydrate, a small organic, an inorganic compound, or an element, for example, gold, iron, or lead. An analyte can be part of a sample that contains other components, or can be the sole or the major component of the sample. An analyte can be a component of a whole cell or tissue, a cell or tissue extract, a fractionated lysate thereof or a substantially purified molecule. In some embodiments, the target analyte is a polypeptide.

The terms “polypeptide” and “protein,” as used herein, generally refer to a polymer of amino acids in which an amino acid may be linked to another amino acid by a peptide bond. In some examples, a polypeptide is a protein. The amino acid may be a naturally occurring amino acid or a non-naturally occurring amino acid (e.g., amino acid analogue). The polymer can be branched and/or can include modified amino acids, and/or may be interrupted by non-amino acids. The polymer may include a plurality of amino acids and/or may have a secondary and/or tertiary structure (e.g., protein). In some examples, the polymer comprises from about 2 to about 50 amino acids, from about 50 to about 100 amino acids, from about 100 to about 1,000 amino acids, from about 1,000 to about 10,000 amino acids, from about 10,000 to about 100,000 amino acids, or more. In some examples, the polymer comprises at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 20 amino acids, at least about amino acids, at least about 40 amino acids, at least about 50 amino acids, at least about 100 amino acids, at least about 1,000 amino acids, at least about 10,000 amino acids, or more amino acids. In some examples, the polymer comprises at most about 10,000 amino acids, at most about 1,000 amino acids, at most about 100 amino acids, at most about 50 amino acids, at most about 40 amino acids, at most about 30 amino acids, at most about 20 amino acids, at most about 10 amino acids, at most about 9 amino acids, at most about 8 amino acids, at most about 7 amino acids, at most about 6 amino acids, at most about 5 amino acids, at most about 4 amino acids, at most about 3 amino acids, at most about 2 amino acids, or less. In some examples, the polymer comprises about 2 amino acids, about 3 amino acids, about 4 amino acids, about 5 amino acids, about 6 amino acids, about 7 amino acids, about 8 amino acids, about 9 amino acids, about 10 amino acids, about 20 amino acids, about 30 amino acids, about 40 amino acids, about 50 amino acids, about 100 amino acids, about 1,000 amino acids, or about 10,000 amino acids. The polymer can have a two-dimensional folded structure and/or three-dimensional folded structure. The protein can comprise one portion that is linear (e.g., primary protein structure) and another portion that is non-linear (e.g., secondary, tertiary, and/or quaternary protein structure). In some cases, the one portion that is linear can comprise from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or about 95% to about 99% of the protein. In some cases, the one portion that is linear can comprise at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more of the protein. In some cases, the one portion that is linear can comprise at most about 99%, at most about 98%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 35%, at most about 30%, at most about 25%, at most about 20%, at most about 15%, at most about 10%, at most about 5%, at most about 1%, or less. In some cases, the one portion that is linear can comprise about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, or about 99%. The protein can be in the form of a polypeptide complex. The polypeptide complex may be a structure formed from two or more polypeptides. The two or more polypeptides in the polypeptide complex may be the same and/or different polypeptides. The protein can be a naturally-occurring protein, a synthetic protein, and/or a recombinant protein. The protein can be naturally-processed in a subject. The protein can be artificially-processed outside a subject. In some cases, the protein can be artificially-processed outside the subject by denaturing the protein (e.g., unfolding the protein). In some cases, the protein can be artificially-processed outside the subject by digesting the protein (e.g., cleaving the protein).

The term “peptide,” as used herein, generally refers to a polymer of amino acids in which an amino acid may be linked to another amino acid by a peptide bond. A protein/polypeptide may be made up of one or more peptides. A peptide can be generated from a protein/polypeptide by cleavage (e.g., digestion). The peptide can be a linear chain of linked amino acids. The peptide may include a plurality of amino acids and/or may have a primary structure. The amino acid may be a naturally occurring amino acid or a non-naturally occurring amino acid (e.g., amino acid analogue). In some examples, the polymer comprises from about 2 to about 50 amino acids, from about 50 to about 100 amino acids, from about 100 to about 1,000 amino acids, from about 1,000 to about 10,000 amino acids, from about 10,000 to about 100,000 amino acids, or more. In some examples, the polymer comprises at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, at least about 20 amino acids, at least about 30 amino acids, at least about 40 amino acids, at least about 50 amino acids, at least about 100 amino acids, at least about 1,000 amino acids, at least about 10,000 amino acids, or more amino acids. In some examples, the polymer comprises at most about 10,000 amino acids, at most about 1,000 amino acids, at most about 100 amino acids, at most about 50 amino acids, at most about 40 amino acids, at most about 30 amino acids, at most about 20 amino acids, at most about 10 amino acids, at most about 9 amino acids, at most about 8 amino acids, at most about 7 amino acids, at most about 6 amino acids, at most about 5 amino acids, at most about 4 amino acids, at most about 3 amino acids, at most about 2 amino acids, or less. In some examples, the polymer comprises about 2 amino acids, about 3 amino acids, about 4 amino acids, about 5 amino acids, about 6 amino acids, about 7 amino acids, about 8 amino acids, about 9 amino acids, about 10 amino acids, about 20 amino acids, about 30 amino acids, about 40 amino acids, about 50 amino acids, about 100 amino acids, about 1,000 amino acids, or about 10,000 amino acids.

The term “amino acid,” as used herein, generally refers to a naturally occurring or non-naturally occurring amino acid (amino acid analogue). The non-naturally occurring amino acid may be a synthesized amino acid. The terms “amino acid sequence,” “peptide sequence,” and “polypeptide sequence,” as used herein, generally refer to at least two amino acids or amino acid analogs that are covalently linked by a peptide (amide) bond or an analog of a peptide bond. The term peptide includes oligomers and/or polymers of amino acids or amino acid analogs. The amino acids of the peptide may be L-amino acids or D-amino acids. A peptide, polypeptide, or protein may be synthetic, recombinant, or naturally occurring. A synthetic peptide and/or protein may be a peptide and or/protein that is produced by artificial approaches in vitro.

As used herein, the term “side chains” or “R” generally refers to unique structures attached to the alpha carbon (attaching the amine and carboxylic acid groups of the amino acid) that render uniqueness to each type of amino acid. R groups have a variety of shapes, sizes, charges, and reactivities, such as charged polar side chains, either positively or negatively charged, such as lysine (+), arginine (+), histidine (+), aspartate (−), and glutamate (−): amino acids can also be basic, such as lysine, or acidic, such as glutamic acid; uncharged polar side chains have hydroxyl, amide, or thiol groups, such as cysteine having a chemically reactive side chain, e.g., a thiol group that can form bonds with another cysteine, serine (Ser) and threonine (Thr), that have hydroxyl R side chains of different sizes; asparagine (Asn), glutamine (Gln), and tyrosine (Tyr); non-polar hydrophobic amino acid side chains include the amino acid glycine, alanine, valine, leucine, and isoleucine having aliphatic hydrocarbon side chains ranging in size from a methyl group for alanine to isomeric butyl groups for leucine and isoleucine; methionine (Met) has a thiol ether side chain; proline (Pro) has a cyclic pyrrolidine side group. Phenylalanine (with its phenyl moiety) (Phe) and tryptophan (Trp) (with its indole group) contain aromatic side chains, which are characterized by bulk as well as lack of polarity.

As used herein, the terms “external amino acid,” “external amino acid residues,” or “exposed amino acid” refers to an amino acid that is solvent-accessible in the native structure of the native protein, non-native protein, non-native polypeptide, and/or non-native peptide. As used herein, the term “internal amino acid,” “internal amino acid residue,” or “buried amino acid” refers to an amino that is solvent-inaccessible in the native structure of the native protein, non-native protein, non-native polypeptide, and/or non-native peptide.

The term “cleavable unit,” as used herein, generally refers to a molecule that can be split into at least two molecules. Non-limiting examples of cleavage reagents and conditions to split a cleavable unit include: enzymes, nucleophilic or basic reagents, reducing agents, photo-irradiation, electrophilic or acidic reagents, organometallic or metal reagents, and oxidizing reagents.

The term “sample,” as used herein, generally refers to a sample containing or suspected of containing a polypeptide. For example, a sample can be a biological sample containing one or more polypeptides. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and food samples (e.g., dairy products, vegetable products, and meat products). The sample may be processed prior to loading into a microfluidic device. For example, the sample may be processed to purify the polypeptides and/or to include reagents.

As used herein, sequencing of peptides “at the single molecule level” generally refers to amino acid sequence information obtained from individual (e.g., single) peptide molecules in a mixture of diverse peptide molecules. The amino acid sequence information may be obtained from an entirety of an individual peptide molecule or one or more portion of the individual peptide molecule, such as a contiguous amino acid sequence of at least a portion of the individual peptide molecule. Alternatively, partial amino acid sequence information may be obtained, which may allow for identification of the peptide or protein. Partial amino acid sequence information, including for example, the pattern of a specific amino acid residue (e.g., lysine) within individual peptide molecules, may be sufficient to uniquely identify or determine an individual peptide molecule. For example, a pattern of amino acids may comprise a plurality of identified positions (e.g., identified as a particular amino acid type, such as lysine, or identified as a particular set of amino acids, such as the set of carboxylate side chain-containing amino acids), and a plurality of unidentified positions. The sequence of identified positions may be searched against a known proteome of a given organism to identify or determine the individual peptide molecule. In some examples, sequencing of a peptide at the single molecule level may identify or determine a pattern of a certain type of amino acid (e.g., lysine) in an individual peptide molecule. Such information may be used to identify or determine a macromolecule (e.g., protein) from which the peptide was derived, without identifying or determining all amino acids of the peptide and/or protein.

As used herein, the term “Edman degradation” generally refers to methods comprising chemical removal of amino acids from peptides or proteins. In some cases, Edman degradation denotes terminal (e.g., N- or C-terminal) amino acid removal. In specific cases, Edman degradation refers to N-terminal amino acid removal through isothiocyanate (e.g., phenyl isothiocyanate) coupling and cyclization with the terminal amine group of an N-terminal residue, such that the N-terminal amino acid is removed from a peptide and/or protein. In some cases, Edman degradation broadly encompasses N-terminal amino acid functionalizations leading to N-terminal amino acid removal. In some cases, Edman degradation encompasses C-terminal amino acid removal. In some cases, Edman degradation comprises terminal amino acid functionalization (e.g., N-terminal amino acid isothiocyanate functionalization) followed by enzymatic removal (e.g., by an ‘Edmanase’ with specificity for chemically derivatized N-terminal amino acids).

As used herein, the term “single molecule sensitivity” generally refers to the ability to acquire data (including, for example, amino acid sequence information) from individual peptide and/or protein molecules in a mixture of diverse peptide and/or protein molecules. In one non-limiting example, the mixture of diverse peptide and/or protein molecules may be immobilized on a solid surface (including, for example, a glass slide, or a glass slide whose surface has been chemically modified). This may include the ability to simultaneously record the fluorescent intensity of multiple individual (e.g., single) peptide and/or protein molecules distributed across the glass surface. Optical devices are commercially available that can be applied in this manner. For example, a conventional microscope equipped with total internal reflection illumination and an intensified charge-couple device (CCD) detector is available. Imaging with a high sensitivity CCD camera allows the instrument to simultaneously record the fluorescent intensity of one or more (e.g., single) peptide and/or protein molecules distributed across a surface. Image collection may be performed using an image splitter that directs light through two band pass filters (one suitable for each fluorescent molecule) to be recorded as two side-by-side images on the CCD surface or two different cameras. In some cases, the imagine is performed with a fluorescent microscope with total internal reflection illuminated and a complementary metal-oxide semiconductor (CMOS) camera. Using a motorized microscope stage with automated focus control to image multiple stage positions in the flow cell may allow millions of one or more single peptides and/or proteins to be sequenced in one experiment.

As used herein, the term “support” generally refers to an entity to which a substance (e.g., molecular construct) can be immobilized. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, a carboxylic acid, or an aldehyde dithiolane. The functional group may couple specifically to an N-terminus or a C-terminus of a peptide and/or protein. The functional group may couple specifically to an amino acid side chain. The functional group may couple to a side chain of an amino acid (e.g., the acid of a glutamate or aspartate, the thiol of a cysteine, the amine of a lysine, or the amide of a glutamine, or asparagine). The functional group may couple specifically to a reactive group on a particular species, such as a label. In some examples of functionalized beads, the functional motif can be reversibly coupled and/or cleaved. A functional motif can also irreversibly couple to a molecule.

As used herein, the term “array” generally refers to a population of sites. Such populations of sites can be differentiated from one another according to relative location. Different molecules that are at different sites of an array can be differentiated from each other according to the locations of the sites in the array. An individual site of an array can include one or more molecules of a particular type. For example, a site can include a single polypeptide and/or peptide having a particular sequence or a site can include several polypeptides and/or peptides having the same sequence. The sites of an array can be different features located on the same substrate. Such features may include, without limitation, wells in a substrate, beads (or other particles) in or on a substrate, projections from a substrate, ridges on a substrate or channels in a substrate. The sites of an array can be separate substrates each bearing at least one molecule. Different molecules attached to separate substrates can be identified according to the locations of the substrates on a surface to which the substrates are associated or according to the locations of the substrates in a liquid or gel. Such different molecules may have the same or different sequences. An array may include one or more wells, and/or an well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group, such as an amino acid side chain, C-terminal carboxylate, or N-terminal amine. The label may comprise at least one reactive group (e.g., a first reactive group and/or a second reactive group). The at least one reactive group may be configured to couple to a polypeptide and/or peptide. The at least one reactive group may be configured to couple to a support. The at least one reactive group may be coupled to or configured to couple to a detectable moiety. A label may provide a measurable signal.

As used herein, the term “polymer matrix” generally refers to a continuous phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and/or crosslinked polymer units. A polymer matrix may also contain non-polymeric species intercalated within its interstitial spaces not occupied by polymer chains. The intercalated species may be solid, liquid or gaseous species. For example, the term ‘polymer matrix’ may encompass desiccated hydrogels, hydrated hydrogels, and/or hydrogels containing glass fibers.

Peptide and/or protein sequence information may be obtained from a polypeptide molecule or from one or more portions of the polypeptide molecule. Peptide and/or protein sequencing may provide complete or partial amino acid sequence information for a peptide and/or a protein sequence or a portion of a peptide and/or a protein sequence. At least a portion of the peptide and/or the protein sequence may be determined at the single molecule level. In some cases, partial amino acid sequence information, including for example, the relative positions of a specific type of amino acid (e.g., lysine) within a peptide and/or a protein or portion of a peptide and/or a protein, may be sufficient to uniquely identify or determine an individual peptide and/or protein molecule. For example, a pattern of amino acids, such as, for example, X-X-X-Lys-X-X-X-X-Lys-X-Lys, which indicates the distribution of lysine molecules within an individual peptide and/or protein molecule, may be searched against a known proteome of a given organism to identify or determine the individual peptide and/or protein molecule. Such information may be used to identify a macromolecule (e.g., protein) from which the peptide was derived, without identifying or determining all amino acids of the peptide and/or protein

Peptide and/protein sequencing may be used to acquire information (including, for example, amino acid sequence information) from individual peptide and/or protein molecules in a mixture of diverse peptide and/or protein molecules. In a non-limiting example, a plurality of peptides and/or proteins may be immobilized on a solid surface (including, for example, a glass slide, or a glass slide whose surface has been chemically modified, a plastic slide, a multi-well plate, a cassette), amino acids from the plurality of peptides and/or proteins may be coupled to fluorescent reporter moieties, and the fluorescent reporter moieties may be optically detected.

In an aspect, the present disclosure provides solutions to the aforementioned challenges by providing expeditious and facile methods for analyzing a polypeptide. Additionally, some aspects of the present disclosure provide compositions that facilitate effective peptide and/or peptide characterization and/or analysis (e.g., determination of protein structure, determination of protein folding). Furthermore, in some aspects, the present disclosure provides kits which enable effective polypeptide analysis.

Method, Systems, and/or Kits

Proteins are the molecular machines of living organisms. When proteins are expressed in the right amounts and/or are folded properly, they may carry on the functions they have in the body. Misfolded proteins that are expressed in a biologically inappropriate amounts may not carry their biological functions and/or lead to diseases. A family of diseases associated directly with misfolding of proteins is proteopathy, also referred to as proteinopathies, protein conformational disorders, or protein misfolding diseases. In proteopathy, often proteins fail to fold into their normal configuration; in this misfolded sate, the proteins can become toxic in some way (e.g., a gain of toxic function) and/or they can lose their normal function.

Protein misfolding may lead to abnormally sticky surfaces on a protein that can interact with other proteins or other misfolded proteins forming aggregates and/or protein complexes. For example, misfolded proteins may have hydrophobic surfaces on their exposed surfaces while hydrophobic moieties may normally be in the core of the proteins. These abnormal protein complexes, interactions, and/or aggregates may render the misfolded protein toxic to the cell, tissue, and/or eventually organs and/or the entire body. For example, in neuronal cells, protein clearance is critical for the maintenance of the integrity of the neurons; abnormal aggregates of misfolded proteins in these cells (e.g., alpha-synuclein, or amyloid beta) may be resistant to protein degradation and/or recycling (e.g., via ubiquitin/proteasome system or autophagy-lysosomal pathway).

In proteopathies, early detection of misfolded proteins, protein aggregates, abnormal protein interactions and/or protein complexes in a patient may help diagnose early onset of the disease. Additionally, quantifying the number of different variations of misfolded proteins, abnormal complexes and/or aggregates can be instrumental in predicting a stage of the disease, and/or identifying or determining appropriate treatments (e.g., choice of drug(s), intensity, or frequency of the treatment).

The present disclosure provides methods, systems, and/or kits for analyzing proteins. Proteins can be in the form of a polypeptide complex (e.g., two or more polypeptides) or one or more polypeptides. Methods, systems, and/or kits of the present disclosure may be used to identify or determine the structure of the one or more polypeptides (e.g., unprocessed proteins, folded proteins). Methods, systems, and/or kits of the present disclosure may be used to identify or determine the two or more polypeptides in the polypeptide complex. The present methods, systems, and/or kits may be used to quantify an amount of the polypeptide complex or the one or more polypeptides. The present methods, systems, and/or kits may also be used to determine the structure (e.g., folding) of the polypeptide complex or the individual polypeptide molecule. Proteins that remain unprocessed (e.g., homogenized, fragmented, digested, denatured, lysed) can be native proteins. Native protein can be folded (e.g., a three-dimensional structure). Native proteins can be properly folded (e.g., functional proteins, healthy) and/or misfolded (e.g., mis-functional, nonfunctional, diseased) As described elsewhere herein, detecting the structure of the polypeptide complex or the individual polypeptide molecule may be used in detecting one or more diseases or disorders (e.g., proteopathies) as well as monitoring their progression and/or treatment.

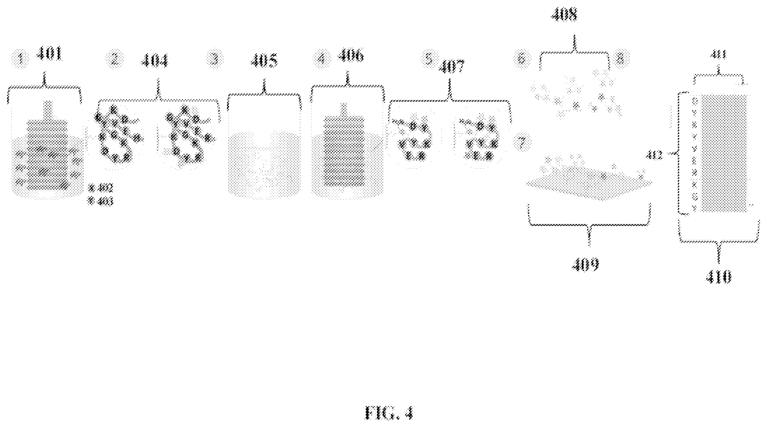

For example, the method of the present disclosure can be depicted by FIG. 4. In some cases, the native protein, non-native protein, non-native polypeptide, and/or non-native peptide can be coupled to a solid 401. The native protein, non-native protein, non-native polypeptide, and/or non-native peptide can comprise one or more external amino acids 402 and/or one or internal amino acids 403. While coupled to the support, the one or more external amino acids on the native protein, non-native protein, non-native polypeptide, and/or non-native peptide can be coupled to one or more labels 404. Subsequent to labeling the one or more external amino acids, the native protein, non-native protein, non-native polypeptide, and/or non-native peptide can be released (e.g., uncoupled) from the support and digested to form one or more peptides and/or proteins 405. The one or more peptides and/or proteins can be coupled to an additional support 406. While coupled to the additional support, the one or more internal amino acids can be labeled with one or more additional labels 407. Subsequent to labeling the one or more additional labels, the one or more peptides and/or proteins can be released from the additional support 408. The one or more peptides and/or proteins are then subsequently analyzed through fluorosequencing 409. The fluorosequencing results can be analyzed to infer the native structure of the native protein, non-native protein, non-native polypeptide, and/or non-native peptide 410. In the analysis, two different data points may be determined for each amino acid residue. The first data point may be the specific amino acid type (e.g., cysteine, lysine, tyrosine, histidine, glutamic acid, aspartic acid) 411. The second data point may be if the amino acid residue was coupled to the one or more labels or the one or more additional labels 412. With the combination of the two data points, the native structure of the native protein, non-native protein, non-native polypeptide, and/or non-native peptide can be determined.

The method, systems, and/or kits described herein may comprise analyzing a biological sample. The biological sample may comprise a molecule (e.g., misfolded protein, properly folded protein) whose presence or absence may be measured or identified. Not meant to be limiting, the biological sample may comprise a macromolecule, such as, for example, a polypeptide or a protein. The biological sample may comprise one or more components (e.g., different polypeptides, heterogenous sample from a cerebrospinal fluid (CSF) of a proteopathy patient). The biological sample may comprise a component of a cell or tissue, a cell or tissue extract, or a fractionated lysate thereof. The biological sample may be purified to have molecules of a single entity (e.g., polypeptide, an oligomer, different oligomers of a polypeptide molecule).

Methods, systems, and/or kits of the present disclosure may comprise isolating, enriching, or purifying a biomolecule, a cell, or tissue from a biological sample. A method, system, and/or kit may utilize a biological sample as a source for a biological species of interest. For example, an assay may derive a protein, such as alpha synuclein, and/or a cell, such as a circulating tumor cell (CTC), from a blood or plasma sample. A method may derive multiple, distinct biological species from a biological sample, such as two separate types of cells. In such cases, the distinct biological species may be separated for analysis (e.g., differently sized alpha synuclein clusters may be segregated for separate analyses) or pooled for common analysis. A biological species may be homogenized, fragmented, or lysed prior to analysis. In some instances, a species or plurality of species from the homogenate, fragmentation products, or lysate may be collected for analysis. For example, a method may comprise collecting CTCs from a buffy coat, optionally isolating individuals CTCs, lysing the CTCs, isolating alpha synuclein clusters from the resulting homogenate, and/or determining the size of the alpha synuclein clusters. A biological species may be artificially unprocessed (e.g., homogenized, fragmented, denatured, digested, or lysed) prior to analysis.

In some cases, one or more polypeptides and/or polypeptide complexes in a sample may be visually detected using a system with a method, system, and/or kit comprising capturing one or more polypeptide complex or polypeptide molecule, labeling the one or more polypeptide complex or polypeptide molecule, and/or detecting the labeled polypeptides.

Numerous commercially available optical devices can be applied in this manner. For example, conventional microscopes equipped with total internal reflection illumination and intensified charge-couple device (CCD) detectors may be adapted for sequencing methods disclosed herein. A high sensitivity CCD camera may be configured to simultaneously record the fluorescence intensity of one or more (e.g., single) peptide and/or protein molecules distributed across a surface, and may be coupled to an image splitter to facilitate the simultaneous collection of multiple, distinct images (e.g., a first image comprising light of a first wavelength and a second image comprising light of a second wavelength). Using a motorized microscope stage with automated focus control to image multiple stage positions in the flow cell may allow thousands or more (e.g., millions) of one or more peptides and/or proteins to be sequenced in a single experiment.

### Protein Structural Analysis

The present disclosure provides methods, systems, and/or kits for analyzing a native protein, non-native protein, non-native polypeptide, and/or non-native peptide (e.g., a native structure). In an aspect, the present disclosure provides a method, comprising: providing a native protein, non-native protein, non-native polypeptide, and/or non-native peptide coupled to a support. In some cases, one or more labels coupled to one or more amino acids of the native protein is detected to identify or determine a native structure of the native protein, non-native protein, non-native polypeptide, and/or non-native peptide using the one or more labels.

The methods provided herein may comprise providing a native protein, —non-native protein, non-native polypeptide, and/or non-native peptide coupled to a support (e.g., solid support). The native protein, non-native protein, non-native polypeptide, and/or non-native peptide can have buried amino acid residues (e.g., internal amino acid residues). In some cases, the buried amino acid residues in the native protein, non-native protein, non-native polypeptide, and/or non-native peptide may not be solvent accessible. The native protein, non-native protein, non-native polypeptide, and/or non-native peptide can have external amino acid residues. In some cases, the external amino acid residues in the native protein, non-native protein, non-native polypeptide, and/or non-native peptide can be solvent accessible. The native protein, non-native protein, non-native polypeptide, and/or non-native peptide may be unprocessed, undigested, and/or non-denatured. The native protein, non-native protein, non-native polypeptide, and/or non-native peptide can be a protein that is not artificially processed (e.g., digested and/or denatured external to a subject). Processing a native protein, non-native protein, non-native polypeptide, and/or non-native peptide can result in the breakage of hydrogen bonding, ionic bonding, dipole-dipole interaction, London dispersion forces, and/or disulfide bonds within the native protein, non-native protein, non-native polypeptide, and/or non-native peptide. An unprocessed protein may be a protein that has not been purified, stained, and/or quantified. For example, a processed protein may have been purified from a sample and/or quantified via colorimetric staining. An undigested protein may be a protein which has not been cleaved. For example, an undigested protein may not have been subjected to conditions (e.g., heat, chemicals, proteases) that may result in cleavage of the protein. A non-denatured protein may be a protein that retains a two-dimensional (2D) structure and/or three-dimensional (3D) structure. In some cases, the non-denatured protein can have a primary protein structure and/or a secondary protein structure. In some cases, the two-dimensional structure and/or the three-dimensional structure can be a folded structure. In some cases, the non-denatured protein can have a tertiary protein structure and/or a quaternary protein sequence. For example, a non-denatured protein may not have been subjected to conditions (e.g., denaturing agents) that cause proteins to unfold. Non-limiting examples of denaturing agents include heat, surface action, ultraviolet light, high pressure, acids, alkalis, heavy metal salts, urea, ethanol, and guanidine detergents The native protein can be folded. The native protein, non-native protein, non-native polypeptide, and/or non-native peptide can be misfolded. In some cases, the native protein, non-native protein, non-native polypeptide, and/or non-native peptide may be a biomarker. The native protein, non-native protein, non-native polypeptide, and/or non-native peptide can be a protein that is naturally processed (e.g., digested and/or denatured internal to a subject). The native protein, non-native protein, non-native polypeptide, and/or non-native peptide can be processed within a subject but not external to a subject. In some cases, the native protein, non-native protein, non-native polypeptide, and/or non-native peptide may not be processed within a subject and/or external to a subject.

The native structure may be a biomarker. —In some cases, the biomarker may be indicative of a disease or a disorder. In some cases, the disease or disorder is a neurodegenerative disease, a synucleinopathy, and/or Tauopathies. In some cases, the disease or disorder may include Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob disease. In some cases, the disease or disorder may include a synucleinopathy associated with aggregation of misfolded alpha-synuclein or formation of misfolded alpha-synuclein oligomers within cells.

In some cases, the disease or disorder may be a cancer. Aberrant α-, β-, γ-synuclein expression can manifest in a wide range of cancers, including a wide range of carcinomas, gangliogliomas, medulloblastomas, neurocytomas, breast, and/or esophageal cancers. Synuclein expression can also contribute to metastasis, and thus can serve as a useful marker for cancer progression. Accordingly, a method, system, and/or kit of the present disclosure may comprise analyzing a cell or tissue sample to identify or determine a cancer state in a subject.

For example, as depicted by FIG. 4, the method herein may comprise a native protein, non-native protein, non-native polypeptide, and/or non-native peptide coupled to a support [401]. The support may be a solid support or a semi-solid support. The solid support or semi-support may be a bead. The bead may be a gel bead. The bead may be a polymer bead. The support may be a resin. The support may comprise, for example, agarose, Sepharose, polystyrene, polyethylene glycol (PEG), or any combination thereof. The support may be a polystyrene bead. The support may be a PEGA resin. The support may be an amino PEGA resin. The support may be a metal core. The bead may be a polymer magnetic bead. The polymer magnetic bead may comprise a metal-oxide. The support may comprise at least one iron oxide core.

The support may be coupled to the support holder at a first position of the support holder. The support holder may be coupled to the support at the first position of the support holder and/or to an additional support comprising an additional biomolecule at a second position of the support holder, or a combination thereof. The coupling may comprise use of a support as described elsewhere herein. The support holder may comprise a lantern bar as described elsewhere herein. The support holder may be configured to couple to a plurality of supports, which supports are each coupled to different additional biomolecules. The support holder may be configured to couple to a plurality of supports, which supports are each coupled to additional biomolecules (e.g., same biomolecules). The support holder may be configured to couple to one support. The support may be coupled to the support holder at a first position of the support holder, the support holder may be coupled to a plurality of additional supports comprising a plurality of additional biomolecules at a plurality of positions of the support holder. The plurality of support may comprises from about 1 to about 2 substrates, about 2 to about 10 substrates, about 10 substrates to about 50 substrates, from about 50 substrates to about 100 substrates, from about 100 substrates to about 200 substrates, from about 200 substrates to about 300 substrates, from about 300 substrates to about 400 substrates, from about 400 substrates to about 500 substrates, from about 500 substrates to about 600 substrates, from about 600 substrates to about 700 substrates, from about 700 substrates to about 800 substrates, from about 800 substrates to about 900 substrates, from about 900 substrates to about 1,000 substrates, or from about 1,000 substrates to about 1,500 substrates. The plurality of supports may comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 32, at least about 35, at least about 40, at least about 45, at least about 48, at least about 50, at least about 60, at least about 70, at least about 80, at least about 90, at least about 96, at least about 100, at least about 150, at least about 192, at least about 200, at least about 250, at least about 300, at least about 350, at least about 384, at least about 400, at least about 500, at least about 600, at least about 700, at least about 800, at least about 900, at least about 1,000, at least about 1,250, at least about 1,500, at least about 1,536, or more substrates. The plurality of supports may comprise at most about 1,536, at most about 1,500, at most about 1,250, at most about 1,000, at most about 900, at most about 800, at most about 700, at most about 600, at most about 500, at most about 400, at most about 384, at most about 350, at most about 300, at most about 250, at most about 200, at most about 192, at most about 150, at most about 100, at most about 96, at most about 90, at most about 80, at most about 70, at most about 60, at most about 50, at most about 48, at most about 45, at most about 40, at most about 35, at most about 32, at most about 30, at most about 25, at most about 20, at most about 19, at most about 18, at most about 17, at most about 16, at most about 15, at most about 14, at most about 13, at most about 12, at most about 11, at most about 10, at most about 9, at most about 8, at most about 7, at most about 6, at most about 5, at most about 4, at most about 3, at most about 2, or fewer substrates. The plurality of support may comprise about 2, about 3, about 4, about 5, about 6, about 7, about 8, about 9, about 10, about 11, about 12, about 13, about 14, about 15, about 16, about 17, about 18, about 19, about 20, about 25, about 30, about 32, about 35, about 40, about 45, about 48, about 50, about 60, about 70, about 80, about 90, about 96, about 100, about 150, about 192, about 200, about 250, about 300, about 350, about 384, about 400, about 500, about 600, about 700, about 800, about 900, about 1,000, about 1,250, about 1,500, or about 1,536. The support may couple to one biomolecule. In some cases, the one biomolecule is a protein. In some cases, the one biomolecule is one or more peptides and/or proteins. The support may couple to one or more biomolecules. In some cases, the one or more biomolecules is one or more proteins and/or one or more peptides.

The support may be a lantern. The lantern may comprise one or more surface linkers as described elsewhere herein. For example, the lantern can be configured with a linker linking the lantern to a biomolecule. In some cases, the lantern can be a synphase lantern. The lantern may comprise a plurality of surfaces held together to increase the surface are available on the lantern. For example, a plurality of rings can be placed in a stacked configuration with small gaps between the rings, and the rings can be linked together in the stack. The lantern may comprise polymers (e.g., plastics, polyethylene, polytetrafluoroethylene, etc.), metals (e.g., iron, steel, aluminum, zinc, etc.), alloys, or the like, or any combination thereof. The lantern may be configured with one or more capture reagents as described elsewhere herein. For example, the lantern can be configured to capture a protein. The lantern may be a substrate. The substrate may comprise a support (e.g., a solid support). The support (e.g., solid support) may, for example a slide, a bead, a well, a pore, or the like, or any combination thereof. The substrate may comprise a polyethylene glycol (PEG) linker, polyacrylate, polyamide, polystyrene, polyethylene, tetrafluoroethylene, or the like, or any combination thereof.

The support may be chemically modified with a capture reagent. The capture reagent can comprise a N-terminal capture reagent. In some cases, the capture reagent binds to the primary amine group on the N-terminal amino acid residue on the native protein, non-native protein, non-native polypeptide, and/or non-native peptide. In some cases, the capture reagent binds to the primary amine group on lysines amino acid residues and/or the thiol group on cysteine amino acid residues on the native protein, non-native protein, non-native polypeptide, and/or non-native peptide. The capture reagent can comprise a C-terminal capture reagent. In some cases, the capture reagent binds to the carboxylic acid on the C-terminal amino acid residue and/or the α-carbon on the C-terminal amino acid residue of the native protein, non-native protein, non-native polypeptide, and/or non-native peptide. The capture reagent can comprise a non-terminal amino acid residue capture reagent. In some cases, the capture reagent binds to the side chains (e.g., R groups) on the non-terminal amino acid residue on the native protein, non-native protein, non-native polypeptide, and/or non-native peptide. In some cases, the capture reagent can react with amine groups on amino acid residues on the native protein, non-native protein, non-native polypeptide, and/or non-native peptide. In some cases, the capture reagent can form an amide bond, a thiourea, and/or cyclic imidazolidinones with the amine groups on amino acid residues on the native protein, non-native protein, non-native polypeptide, and/or non-native peptide. In some cases, the capture reagent can react with thiol groups on amino acid residues on the native protein, non-native protein, non-native polypeptide, and/or non-native peptide. In some cases, the capture reagent can form a thioether bond with the thiol groups on amino acid residues on the native protein, non-native protein, non-native polypeptide, and/or non-native peptide. The capture reagent can comprise a reversal capture reagent (e.g., couple and couple without cleavage). The capture reagent can comprise a nonreversible capture reagent. The capture reagent can be a substituted anhydride, an aldehyde group, an isothiocyanate group, a maleimide group, a methylene pyrrolone group, a succinimidyl ester group, polyacrylate, polyamide, and/or polystyrene. The capture reagent may be a substituted anhydride. The substituted anhydride can be dialkyl maleic anhydride, maleic anhydride, and/or alkenyl succinic anhydride. The capture reagent can be an aldehyde (e.g., isomeric pyridinaldehyde). The aldehyde can be pyridinecarbaldehyde (PCA), pyridine-3-carboxaldehyde and/or pyridine-4-carboxaldehyde. The capture reagent may be an isothiocyanate group. The isothiocyanate can be benzyl isothiocyanate, allyl isothiocyanate, cyclohexyl isothiocyanate, and/or phenyl isothiocyanate.

In some cases, the capture reagent couples to the native protein, non-native protein, non-native polypeptide, and/or non-native peptide through a reaction between an acid anhydride and an amine. The support may comprise the acid anhydride coupled to an amine group on the native protein, non-native protein, non-native polypeptide, and/or non-native peptide. The native protein, non-native protein, non-native polypeptide, and/or non-native peptide may comprise at the amine at terminal and/or non-terminal amino acid residues. In some cases, the reaction between the acid anhydride and the amine can form an amide. In some cases, the reaction between the acid anhydride and the amine can be an aminolysis of an acid anhydride. In some cases, the aminolysis of the acid anhydride can be an aminolysis of a cyclic anhydride. In some cases, the aminolysis of the acid anhydride can be an aminolysis of a 2,5-furanedionyl moiety (e.g., maleic anhydride). In some cases, the 2,5-furanedionyl moiety can be substituted or unsubstituted. In some cases, the aminolysis of the acid anhydride can be an aminolysis of a citraconic anhydride moiety. The reaction between the acid anhydride and the amine can be an anhydride-like reaction In some cases, the anhydride-like reaction can be a nucleophilic reaction. In some cases, the anhydride-like reaction can further comprise deprotonation and leaving group removal. The anhydride-like reaction can reversibly couple the capture reagent to the native protein, non-native protein, non-native polypeptide, and/or non-native peptide. In some cases, the anhydride-like reaction can be a citraconic anhydride-like reaction. In some cases, the support can be modified to express a spacer. In some cases, the spacer couples the capture reagent to the support. In some cases, the spacer can comprise bond (e.g., covalent and/or non-covalent). In some cases, the spacer can comprise a cleavable linker. The cleavable linker may be an enzymatically cleavable linker (e.g., Mal-PEG1-Val-Cit-PAB-OH, Alkyne-Val-Cit-PAB-FAM). The cleavable linker can comprise a disulfide linker (e.g., aminoethyl-SS-ethylalcohol, Azido-SS-PEG2-acid, azido-phenyl-amido-S—S-Sulfo-NHS, propargyl-PEG1-SS-PEG1-propargyl, DBCO-S-S-acidtrifluoroacetamidoethyl-SS-propionic NHS ester). The cleavable linker may be a chemically cleavable linker. Non-limiting examples of chemically cleavage linkers include Trityl (e.g., Chlorotrityl), 4-(hydroxymethyl)benzoic acid (HMBA), Dde linker (N-1-(4,4-dimethyl-2,6-dioxocyclohex-1-ylidene)-3-ethyl), carboxamide, N-alkylamide, ester, thioester, hydrazide, alcohols, and aldehydes. The cleavable linker may be a photocleavable linker (e.g., 2-nitrobenzyl, α-thioacetophenone, 4-azide-TFP-Amide-SS-propionic acid, PC Azido-PEG3-NHS carbonate ester). The cleavable linker may be capable of being cleaved by a change in pH. The cleavable linker may comprise an aldehyde. The aldehyde linker may be pyridinecarbaldehyde (PCA) linker or a derivative of PCA. The caboxamide linker may be a Rink amide linker and/or a sieber amide linker. The N-alkylamide linker may be a methyl indole AM linker. The ester linker may be a HMBA-PEGA linker. The thiol linker may be a 4-sulfamylbutyryl linker. The alcohol linker may be a HMBA-PEGA linker.

The spacer can be flexible, rigid, and/or cleavable. The spacer can comprise a polymeric spacer, a polyether spacer, and/or an alkyl spacer (e.g., methane, ethane, propane, butane). In some cases, the spacer can comprise a bond (e.g., a covalent bond). The spacer can comprise polyethylene glycol (PEG) groups, alkyl groups, and/or amino acid residues. In some cases, the spacer can have from about 1 PEG group to about 10 PEG groups. In some cases, the spacer can have at least about 1 PEG group, at least about 2 PEG groups, at least about 3 PEG groups, at least about 4 PEG groups, at least about 5 PEG groups, at least about 6 PEG groups, at least about 7 PEG groups, at least about 8 PEG groups, at least about 9 PEG groups, at least about 10 PEG groups, or more. In some cases, the spacer can have at most about 10 PEG groups, at most about 9 PEG groups, at most about 8 PEG groups, at most about 7 PEG groups, at most about 6 PEG groups, at most about 5 PEG groups, at most about 4 PEG groups, at most about 3 PEG groups, at most about 2 PEG groups, at most about 1 PEG groups, or less. In some cases, the spacer can have about 1 PEG group, about 2 PEG groups, about 3 PEG groups, about 4 PEG groups, about 5 PEG groups, about 6 PEG groups, about 7 PEG groups, about 8 PEG groups, about 9 PEG groups, or about 10 PEG groups. In some cases, the spacer can have from about 1 to about 10 alkyl groups. In some cases, the spacer can have at least about 1 alkyl group, at least about 2 alkyl groups, at least about 3 alkyl groups, at least about 4 alkyl groups, at least about 5 alkyl groups, at least about 6 alkyl groups, at least about 7 alkyl groups, at least about 8 alkyl groups, at least about 9 alkyl groups, at least about 10 alkyl groups, or more. In some cases, the spacer can have at most about 10 alkyl groups, at most about 9 alkyl groups, at most about 8 alkyl groups, at most about 7 alkyl groups, at most about 6 alkyl groups, at most about 5 alkyl groups, at most about 4 alkyl groups, at most about 3 alkyl groups, at most about 2 alkyl groups, at most about 1 alkyl group, or less. In some cases, the spacer can have about 1 alkyl group, about 2 alkyl groups, about 3 alkyl groups, about 4 alkyl groups, about 5 alkyl groups, about 6 alkyl groups, about 7 alkyl groups, about 8 alkyl groups, about 9 alkyl groups, or about 10 alkyl groups. In some cases, the spacer can have from about 1 amino acid residue to about 10 amino acid residues. In some cases, the spacer can have at least about one amino acid residues, at least about two amino acid residues, at least about three amino acid residues, at least about four amino acid residues, at least about five amino acid residues, at least about six amino acid residues, at least about seven amino acid residues, at least about eight amino acid residues, at least about nine amino acid residues, at least about ten amino acid residues, or more. In some cases, the spacer can have at most about ten amino acid residues, at most about nine amino acid residues, at most about eight amino acid residues, at most about seven amino acid residues, at most about six amino acid residues, at most about five amino acid residues, at most about four amino acid residues, at most about three amino acid residues, at most about two amino acid residues, at most about one amino acid residue, or less. In some cases, the spacer can have about one amino acid residue, about two amino acid residues, about three amino acid residues, about four amino acid residues, about five amino acid residues, about six amino acid residues, about seven amino acid residues, about eight amino acid residues, about nine amino acid residues, or about ten amino acid residues. The amino acid residues in the spacer can be any of the amino acid residues of the present disclosure.

In some cases, the cleavable linker may be attached to the native protein, non-native protein, non-native polypeptide, and/or non-native peptide by a first linking group. The first linking group can comprise a capture reagent. In some cases, the spacer may be attached to the native protein, non-native protein, non-native polypeptide, and/or non-native peptide and/or support by a second linking group. In some cases, the spacer may be attached to the native protein, non-native protein, non-native polypeptide, and/or non-native peptide and/or support by a third linking group. In some cases, the cleavable linker may be attached to the native protein, non-native protein, non-native polypeptide, and/or non-native peptide and/or support by a fourth linking group. In some cases, the cleavable linker may be attached to the native protein, non-native protein, non-native polypeptide, and/or non-native peptide and/or support by a fifth linking group. In some cases, the cleavable linker may be attached to the native protein, non-native protein, non-native polypeptide, and/or non-native peptide and/or support by a sixth linking group. In some cases, the cleavable linker may be attached to the native protein, non-native protein, non-native polypeptide, and/or non-native peptide and/or support by a seventh linking group. In some cases, the cleavable linker may be attached to the native protein, non-native protein, non-native polypeptide, and/or non-native peptide and/or support by an eighth linking group. In some cases, the cleavable linker may be attached to the native protein, non-native protein, non-native polypeptide, and/or non-native peptide and/or support by a ninth linking group. In some cases, the cleavable linker may be attached to the native protein, non-native protein, non-native polypeptide, and/or non-native peptide and/or support by a tenth linking group. The first linking group, the second linking group, the third linking group, the fourth linking group, the fifth linking group, the sixth linking group, the seventh linking group, the eighth linking group, the ninth linking group, and/or the tenth linking group can be an additional spacer.

The methods provided herein may comprise providing a native protein, non-native protein, non-native polypeptide, and/or non-native peptide coupled to a support, wherein the native protein, non-native protein, non-native polypeptide, and/or non-native peptide comprises a native structure. The native structure can comprise a secondary protein structure, such as, for example, α-helices and/or β-sheets. The native structure can comprise a tertiary protein structure (e.g., a three-dimensional arrangement of a polypeptide chain). The tertiary protein structure may involve hydrophobic interactions, hydrogen bonds, salt bridges, and/or sulfur-sulfur covalent bonds. In some cases, the tertiary structure is maintained at physiological conditions (e.g., temperature from about 20 to about 40° Celsius (C), pH from about 6 to about 8, and/or atmospheric oxygen concentration). In some cases, the physiological conditions can be a temperature. In some cases, the temperature can be from about 20° C. to about 25° C., from about 25° C. to about 30° C., from about 30° C. to about 35° C., or from about 35° C. to about 40° C. In some cases, the temperature can be at least about 20° C., at least about 25° C., at least about 30° C., at least about 35° C., at least about 40° C., or more. In some cases, the temperature can be at most about 40° C., at most about 35° C., at most about 30° C., at most about 25° C., at most about 20° C., or less. In some cases, the temperature can be about 20° C., about 25° C., about 30° C., about 35° C., or about 40° C. In some cases, the physiological conditions can be a pH. In some cases, the pH can be from about 6 to about 6.5, from about 6.5 to about 7, from about 7 to about 7.5, or from about 7.5 to about 8. In some cases, the pH can be at least about 6, at least about 6.25, at least about 6.5, at least about 6.75, at least about 7, at least about 7.25, at least about 7.5, at least about 7.75, at least about 8, or more. In some cases, the pH can be at most about 8, at most about 7.75, at most about 7.5, at most about 7.25, at most about 7, at most about 6.75, at most about 6.5, at most about 6.25, at most about 6, or less. In some cases, the pH can be about 6, about 6.25, about 6.5, about 6.75, about 7, about 7.25, about 7.5, about 7.75, or about 8. In some cases, the physiological conditions can be atmospheric oxygen concentration. In some cases, the atmospheric oxygen concentration can be from about 15% to about 20%, from about 20% to about 25%, or from about 25% to about 30%. In some cases, the atmospheric oxygen concentration can be at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, or more. In some cases, the atmospheric oxygen concentration can be at most about 30%, at most about 29%, at most about 28%, at most about 27%, at most about 26%, at most about 25%, at most about 24%, at most about 23%, at most about 22%, at most about 21%, at most about 20%, at most about 19%, at most about 18%, at most about 17%, at most about 16%, at most about 15%, or less. In some cases, the atmospheric oxygen concentration can be about 15%, about 16%, about 17%, about 18%, about 19%, about 20%, about 21%, about 22%, about 23%, about 24%, about 25%, about 26%, about 27%, about 28%, about 29%, or about 30%.

The native structure can comprise a quaternary structure (e.g., two or more proteins associated with each other). The two or more proteins may be the same protein (e.g., fibrils or aggregates). The two or more proteins may be different proteins and/or the two or more different proteins may form a protein complex. The quaternary structure may be stabilized through cross-linking between the two or more proteins (e.g., intermolecular disulfide bonds and/or intramolecular disulfide bonds).

The native structure can be present in solution at neutral pH (e.g., pH from about 6 to about 8) and/or physiological conditions. The neutral pH can be from about 6 to about 7 or from about 7 to about 8. The neutral pH can be at least about 6, at least about 6.25, at least about 6.5, at least about 6.75, at least about 7, at least about 7.25, at least about 7.5, at least about 7.75, at least about 8, or more. The neutral pH can be at most about 8, at most about 7.75, at most about 7.5, at most about 7.25, at most about 7, at most about 6.75, at most about 6.5, at most about 6.25, at most about 6, or less. The neutral pH can be about 6, about 6.25, about 6.5, about 6.75, about 7, about 7.25, about 7.5, about 7.75, or about 8. The native protein, non-native protein, non-native polypeptide, and/or non-native peptide can retain a biologically active structure (e.g., enzymatic and/or catalytic activity). The biologically active structure may retain structural form (e.g., a fibrillar structure). Non-limiting examples of proteins with fibrillar structure include collagen, α-keratin, elastin, resilin, fibrinogen, and myosin heavy chain.

The methods provided herein may comprise providing a native protein, non-native protein, non-native polypeptide, and/or non-native peptide coupled to a surface, wherein the native protein, non-native protein, non-native polypeptide, and/or non-native peptide can comprise one or more internal amino acids (e.g., buried amino acid residues) and/or one or more external amino acids (e.g., surface and/or surface-exposed amino acid residues), for example, as depicted in [403] in FIG. 4. The surface-exposed amino acid residues may comprise amino acids with R groups (e.g., amino acid side chains) accessible to a solvent. The buried amino acid residues may comprise amino acids with R groups inaccessible to the solvent. Accessibility of the one or more internal amino acids and/or the one or more external amino acids on the native protein, non-native protein, non-native polypeptide, and/or non-native peptide can be determined in solution phase under physiological conditions. Accessibility of amino acid residues in native protein, non-native protein, non-native polypeptide, and/or non-native peptide can be approximated through a solvent accessibility model, such as, for example, as described in Moelbert, Susanne, Eldon Emberly, and Chao Tang. “Correlation between sequence hydrophobicity and surface-exposure pattern of database proteins.” Protein Science 13.3 (2004): 752-762, which is hereby incorporated by reference. In some cases, the solvent accessibility model quantifies the degree to which the hydrophobicity sequence of a protein correlates with its pattern of surface exposure based on the known protein structure.

The native protein, non-native protein, non-native polypeptide, and/or non-native peptide can comprise a plurality of amino acids. The native protein, non-native protein, non-native polypeptide, and/or non-native peptide may comprise amino acids that are L-amino acids or D-amino acids. A native protein, non-native protein, non-native polypeptide, and/or non-native peptide may be synthetic, recombinant, or naturally occurring. A synthetic native protein, non-native protein, non-native polypeptide, and/or non-native peptide may be a native protein, non-native protein, non-native polypeptide, and/or non-native peptide that is produced by artificial approaches in vitro. A recombinant native protein, non-native protein, non-native polypeptide, and/or non-native peptide may be native protein, non-native protein, non-native polypeptide, and/or non-native peptide that is encoded by a gene (e.g., recombinant DNA) that has been cloned into a system that supports expression of the gene and/or translation of messenger RNA (e.g, mRNA). A naturally occurring native protein, non-native protein, non-native polypeptide, and/or non-native peptide may be a native protein, non-native protein, non-native polypeptide, and/or non-native peptide that is produced in vivo from a subject's genome. In some cases, the native protein, non-native protein, non-native polypeptide, and/or non-native peptide can comprise at least one natural amino acid. A natural amino acid can comprise an amino acid that is incorporated into a protein and/or peptide through the translation process. In some cases, the natural amino acid can comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. At least one amino acid of the plurality of amino acids may be selected from the group consisting of lysine, cysteine, glutamic acid, aspartic acid, tyrosine, arginine, histidine, threonine, serine, glutamine, asparagine and tryptophan. The plurality of amino acids may comprise one or more amino acids, the one or more amino acid selected from the group consisting of lysine, cysteine, glutamic acid, aspartic acid, tyrosine, arginine, histidine, threonine, serine, glutamine, asparagine and tryptophan. The native protein, non-native protein, non-native polypeptide, and/or non-native peptide may comprise one amino acid selected from the group consisting of lysine, cysteine, glutamic acid, aspartic acid, tyrosine, arginine, histidine, threonine, serine, glutamine, asparagine and tryptophan. In some cases, the native protein, non-native protein, non-native polypeptide, and/or non-native peptide can comprise at least one non-natural amino acid. A non-natural amino acid can be an amino acid that is not naturally incorporated into proteins through the translation process. Non-limiting examples of non-natural amino acids include hydroxyproline, beta-alanine, citrulline, ornithine, norleucine, 3-nitrotyrosine, nitroarginine, pyroglutamic acid, 4-aminobenzoic acid, gamma-aminobutyric acid, aminoisobutyric acid, dehydroalanine, cystine, cystathionine, lanthionine, diaminopimelic acid, alloisoleucine, norvaline, and sarcosine. The plurality of amino acids may comprise a non-natural amino acid. The plurality of amino acids may comprise a D-amino acid.

The native protein, non-native protein, non-native polypeptide, and/or non-native peptide can comprise all natural amino acids. The native protein, non-native protein, non-native polypeptide, and/or non-native peptide can comprise all non-natural amino acids. The native protein, non-native protein, non-native polypeptide, and/or non-native peptide can comprise a portion of natural amino acids and another portion of non-natural amino acids. In some cases, the native protein, non-native protein, non-native polypeptide, and/or non-native peptide can comprise from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100% natural amino acids. In some cases, the native protein, non-native protein, non-native polypeptide, and/or non-native peptide can comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more natural amino acids. In some cases, the native protein, non-native protein, non-native polypeptide, and/or non-native peptide can comprise at most about 99%, at most about 98%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, at most about 1%, or less natural amino acids. In some cases, the native protein, non-native protein, non-native polypeptide, and/or non-native peptide can comprise about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, or about 100% natural amino acids.

In some cases, the native protein, non-native protein, non-native polypeptide, and/or non-native peptide can comprise from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100% non-natural amino acids. In some cases, the native protein, non-native protein, non-native polypeptide, and/or non-native peptide can comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more non-natural amino acids. In some cases, the native protein, non-native protein, non-native polypeptide, and/or non-native peptide can comprise at most about 99%, at most about 98%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, at most about 1%, or less non-natural amino acids. In some cases, the native protein, non-native protein, non-native polypeptide, and/or non-native peptide can comprise about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99%, or about 100% non-natural amino acids.

The methods provided herein may comprise providing a native protein, non-native protein, non-native polypeptide, and/or non-native peptide coupled to a support, wherein the native protein, non-native protein, non-native polypeptide, and/or non-native peptide comprises one or more labels. The one or more labels may be coupled to a terminal end of the native protein, non-native protein, non-native polypeptide, and/or non-native peptide. The terminal end can comprise the N-terminal amino acid residue and/or the C-terminal amino acid residue. The one or more labels may be coupled to a non-terminal amino acid on the native protein, non-native protein, non-native polypeptide, and/or non-native peptide. The non-terminal amino acid can comprise any residue on the native protein, non-native protein, non-native polypeptide, and/or non-native peptide excluding the N-terminal residue and/or the C-terminal residue. In some cases, the one or more external amino acids can comprise terminal amino acid residues (e.g., N-terminal amino acid residue and/or C-terminal amino acid residue) and/or non-terminal amino acids. In some cases, the one or more external amino acids can comprise the C-terminal residue. In some cases, the one or more external amino acids can comprise the N-terminal residue. In some cases, the one or more external amino acids can comprise the N-terminal residue and/or the C-terminal residue. In some cases, the one or more external amino acids can comprise non-terminal amino acids. In some cases, the one or more labels may not alter the native structure of the native protein, non-native protein, non-native polypeptide, and/or non-native peptide when coupled to the one or more external amino acids.

The one or more labels may emit a detectable signal. A signal may be optical, chemical (e.g., change in pH), radiometric (e.g., isotopic signals), electronic (e.g., disruption of ionic current), informational (e.g., binary patterns), or a combination thereof. An optical signal may be luminescent (e.g., chemiluminescent, bioluminescent, electroluminescent, sonoluminescent, photoluminescent, radioluminescent, or thermoluminescent). Some examples of photoluminescent optical signals include fluorescent or phosphorescent signals. An optical signal may come from a chromophore (e.g., a fluorophore, fluorescent dye). An optical signal may be any molecule, macromolecule, or molecular construct capable of emitting photons. Optical signals may be emitted in response to excitation. Optical signals may be differentiable from one another, such as by color. In some examples, multiple optical signals can be used within a single system, method, or kit. For example, one or more of fluorophores, some or all of which being capable of emitting a differentiable optical signal can be provided. A plurality of optical signals may include, for example, multiple colors. In some cases, fluorescent dyes that produce one color, two colors, three colors, four colors, five colors, or more can be provided. In some cases, fluorescent dyes that produce 20 colors or more can be provided. Fluorophores may comprise one or more classes of dyes such as rhodamine, cyanine or carbopyronine dye (Atto647N). Fluorophores may include, for example, a fluorophore-iodoacetamide (e.g., Atto647-Iodoacetamide); a fluorophore-succinimidyl ester) e.g., Atto647N-NHS), a fluorophore-amine (e.g., Atto6-(7N-Amine), a dithiolane-fluorophore (e.g., a custom synthesized fluorophore, an oxidized dithiolane-fluorophore, a reduce dithiolane-fluorophore), a fluorophore-Azide (e.g., Atto647N-Azide), Oregon Green (OG)-iodoacetamide, OG488-NHS, OG488-Tetrazine, OG514-NHS, Janelia Fluor (JF)-NHS, JF-FreeAcid, JF-Azide, JF-Dithiolane, Atto647N-Alkyne, Atto647N-FreeAcid, Atto425-NHS, Atto425-FreeAcid, Atto425-Amine, Atto425-Azide, Atto425-DBCO, SF554-NHS, azide-DBCO, methyltetrazine-norbornene, aldehyde-dithiolane, aryl iodide-boronic acid, or TexasRed-NHS. Optical signals may also comprise an absence or a loss of an optical signal or a change in optical signal (e.g., FRET, BRET, homo-FRET, or other energy transfer luminescence, such as Alexa fluors, BODIPY dyes, Xanthene dues, or Cyanine dyes).

The native protein, non-native protein, non-native polypeptide, and/or non-native peptide can comprise one or more labels coupled to the one or more external amino acids on the native protein, non-native protein, non-native polypeptide, and/or non-native peptide, for example, as depicted by in FIG. 4. In some cases, the native protein, non-native protein, non-native polypeptide, and/or non-native peptide can comprise one or more labels coupled to the one or more external amino acids and the one or more internal amino acids on the native protein non-native protein, non-native polypeptide, and/or non-native peptide. The one or more labels may comprise an optical label (e.g., fluorescent protein or peptide and/or fluorescent dye). The one or more labels may be directly coupled to the one or more external amino acids on the native protein non-native protein, non-native polypeptide, and/or non-native peptide, such as, for example, through a covalent bond (e.g., a peptide bond). The one or more labels may be directly coupled to the one or more external amino acids on the native protein non-native protein, non-native polypeptide, and/or non-native peptide, such as, for example through a non-covalent bond (e.g., an electrostatic interaction, a hydrogen bond, a van der Waals interaction, and/or a hydrophobic interaction). The one or more labels may be irreversibly or reversibly coupled to the one or more external amino acids on the native protein non-native protein, non-native polypeptide, and/or non-native peptide. The one or more labels may be indirectly coupled to the one or more external amino acids on the native protein non-native protein, non-native polypeptide, and/or non-native peptide. The one or more external amino acids may be coupled to a first reactive group (e.g., Click). The first reactive group can be directly coupled to the side chain of the one or more external amino acids on the native protein non-native protein, non-native polypeptide, and/or non-native peptide, such as, for example, through a covalent bond (e.g., a peptide bond). The first reactive group can be directly coupled to the side chain of the one or more external amino acids on the native protein non-native protein, non-native polypeptide, and/or non-native peptide, such as, for example, through a non-covalent bond (e.g., an electrostatic interaction, a hydrogen bond, a van der Waals interaction, and/or a hydrophobic interaction). The first reactive group may be irreversibly or reversibly coupled to the one or more external amino acids on the native protein non-native protein, non-native polypeptide, and/or non-native peptide. In some cases, the first reactive group is coupled to the one or more labels (e.g., Clack, a second reactive group). The first reactive group may be directly coupled to the one or more labels, such as, for example, through a covalent bond (e.g., peptide bond). The first reactive group may be directly coupled to the one or more labels, such as, for example, through a non-covalent bond (e.g., an electrostatic interaction, a hydrogen bond, a van der Waals interaction, and/or a hydrophobic interaction. The first reactive group can be irreversibly or reversibly coupled to the one or more labels.

The one or more labels can couple to acidic amino acid residues (e.g., glutamic acid and/or aspartic acid), histidine amino acid residues, and/or tyrosine amino acid residues. In some cases, the coupling the one or more labels to the one or more external amino acids on the native protein, non-native protein, non-native polypeptide, and/or non-native peptide is subsequent to providing a native protein, non-native protein, non-native polypeptide, and/or non-native peptide coupled to a support. In some cases, the coupling the one or more labels to the one or more external amino acids on the native protein, non-native protein, non-native polypeptide, and/or non-native peptide is prior to providing a native protein, non-native protein, non-native polypeptide, and/or non-native peptide coupled to a support.

In some cases, the native protein, non-native protein, non-native polypeptide, and/or non-native peptide may be coupled to the support prior to coupling the one or more labels to the one or more external amino acids of the native protein, non-native protein, non-native polypeptide, and/or non-native peptide. In some cases, the native protein, non-native protein, non-native polypeptide, and/or non-native peptide may be coupled to the support subsequent to coupling the one or more labels to the one or more external amino acids of the native protein, non-native protein, non-native polypeptide, and/or non-native peptide. The native protein, non-native protein, non-native polypeptide, and/or non-native peptide may be coupled directly to the support, such as, for example through a covalent bond (e.g., a peptide bond). The native protein, non-native protein, non-native polypeptide, and/or non-native peptide may be coupled directly to the support, such as, for example, through a non-covalent bond (e.g., an electrostatic interaction, a hydrogen bond, a van der Waals interaction, and/or a hydrophobic interaction).

Alternatively, the native protein, non-native protein, non-native polypeptide, and/or non-native peptide can be cross-linked in solution. In some cases, the cross-linking of the native protein, non-native protein, non-native polypeptide, and/or non-native peptide can allow the native protein, non-native protein, non-native polypeptide, and/or non-native peptide to be inert in the solution. In some cases, the native protein, non-native protein, non-native polypeptide, and/or non-native peptide can be cross-linked with formaldehyde, bismaleimidohexane (BMH), sulfosuccinimidyl 6-[3′-(2-pyridyldithio) propionamido]hexanoate (Sulfo-LC-SPDP), Sulfo-SMCC, N-hydroxysuccinimide esters (NHS esters), N-hydroxysuccinimide imidoester, glutaraldehyde, dimethylpimelimidate, disuccinimidyl sufoxide (DSSO), and/or disuccinimidyl dibutyric urea (USBU). The one or more external amino acids on the native protein, non-native protein, non-native polypeptide, and/or non-native peptide can be coupled to the one or more labels while the native protein, non-native protein, non-native polypeptide, and/or non-native peptide is cross-linked. In some cases, the cross-linking can be reversed with heat (e.g., about 70° C. to about 100° C.). In some cases, the cross-linking can be reversed at a temperature from about 70° C. to about 80° C., from about 80° C. to about 90° C., or from about 90° C. to about 100° C. In some cases, the cross-linking can be reversed at a temperature of at least about 70° C., at least about 75° C., at least about 80° C., at least about 85° C., at least about 90° C., at least about 95° C., at least about 100° C., or more. In some cases, the cross-linking can be reversed at a temperature of at most about 100° C., at most about 95° C., at most about 90° C., at most about 85° C., at most about 80° C., at most about 75° C., at most about 70° C., or less. In some cases, the cross-linking can be reversed at a temperature of about 70° C., about 75° C., about 80° C., about 85° C., about 90° C., about 95° C., or about 100° C.

The method can further comprise one or more washing operations. In some cases, the one or more washing operations can eliminate non-target species (e.g., lipids, sugars, minerals, vitamins, nucleic acids) and/or metabolites. In some cases, the one or more washing operations can remove excess reagents and/or labels. In some cases, the method can comprise from about one to about two wash operations, from about two to about three wash operations, from about three to about four wash operations, from about four wash operations to about five wash operations, from about five wash operations to about six wash operations, from about six wash operations to about seven wash operations, from about seven wash operations to about eight wash operations, from about eight wash operations to about nine wash operations, or from about nine wash operations to about ten wash operations. In some cases, the method can comprise at least about one wash operation, at most about two wash operations, at least about three wash operations, at least about four wash operations, at least about five wash operations, at least about six wash operations, at least about seven wash operations, at least about eight wash operations, at least about nine wash operations, at least about ten wash operations or more. In some cases, the method can comprise at most about ten wash operations, at most about nine wash operations, at most about eight wash operations, at most about seven wash operations, at most about six wash operations, at most about five wash operations, at most about four wash operations, at most about three wash operations, at most about two wash operations, at most about one wash operation, or less. In some cases, the method can comprise about one wash operation, about two wash operations, about three wash operations, about four wash operations, about five wash operations, about six wash operations, about seven wash operations, about eight wash operations, about nine wash operations, or about ten wash operations.

In some cases, the native protein, non-native protein, non-native polypeptide, and/or non-native peptide comprising the one or more external amino acids coupled to the one or more labels may be released from the support (e.g., subsequent to providing a native protein, non-native protein, non-native polypeptide, and/or non-native peptide coupled to a support), for example, as depicted as in FIG. 4. A low pH (e.g., pH from about 2 to about 5) can release the native protein, non-native protein, non-native polypeptide, and/or non-native peptide from the support. In some cases, the low pH can be from about 2 to about 3, from about 3 to about 4, from about 4 to about 5. In some cases, the low pH can be at least about 2, at least about 2.1, at least about 2.2, at least about 2.3, at least about 2.4, at least about 2.5, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, at least about 5, or more. In some cases, the low pH can be at most about 5, at most about 4.9, at most about 4.8, at most about 4.7, at most about 4.6, at most about 4.5, at most about 4.4, at most about 4.3, at most about 4.2, at most about 4.1, at most about 4, at most about 3.9, at most about 3.8, at most about 3.7, at most about 3.6, at most about 3.5, at most about 3.4, at most about 3.3, at most about 3.2, at most about 3.1, at most about 3, at most about 2.9, at most about 2.8, at most about 2.7, at most about 2.6, at most about 2.5, at most about 2.4, at most about 2.3, at most about 2.2, at most about 2.1, at most about 2, or less. In some cases, the low pH can be about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5. A high pH (e.g., pH from about 9 to about 14) can release the native protein, non-native protein, non-native polypeptide, and/or non-native peptide from the support. In some cases, the high pH can be from about 9 to about 10, from about 10 to about 11, from about 11 to about 12, from about 12 to about 13, from about 13 to about 14. In some cases, the high pH can be at least about 9, at least about 9.1, at least about 9.2, at least about 9.3, at least about 9.4, at least about 9.5, at least about 9.6, at least about 9.7, at least about 9.8, at least about 9.9, at least about 10, at least about 10.1, at least about 10.2, at least about 10.3, at least about 10.4, at least about 10.5, at least about 10.6, at least about 10.7, at least about 10.8, at least about 10.9, at least about 11, at least about 11.1, at least about 11.2, at least about 11.3, at least about 11.4, at least about 11.5, at least about 11.6, at least about 11.7, at least about 11.8, at least about 11.9, at least about 12, at least about 12.1, at least about 12.2, at least about 12.3, at least about 12.4, at least about 12.5, at least about 12.6, at least about 12.7, at least about 12.8, at least about 12.9, at least about 12, at least about 12.1, at least about 12.2, at least about 12.3, at least about 12.4, at least about 12.5, at least about 12.6, at least about 12.7, at least about 12.8, at least about 12.9, at least about 13, at least about 13.1, at least about 13.2, at least about 13.3, at least about 13.4, at least about 13.5, at least about 13.6, at least about 13.7, at least about 13.8, at least about 13.9, at least about 14, or more. In some cases, the high pH can be at most about 14, at most about 13.9, at most about 13.8, at most about 13.7, at most about 13.6, at most about 13.5, at most about 13.4, at most about 13.3, at most about 13.2, at most about 13.1, at most about 13, at most about 12.9, at most about 12.8, at most about 12.7, at most about 12.6, at most about 12.5, at most about 12.4, at most about 12.3, at most about 12.2, at most about 12.1, at most about 12, at most about 11.9, at most about 11.8, at most about 11.7, at most about 11.6, at most about 11.5, at most about 11.4, at most about 11.3, at most about 11.2, at most about 11.1, at most about 11, at most about 10.9, at most about 10.8, at most about 10.7, at most about 10.6, at most about 10.5, at most about 10.4, at most about 10.3, at most about 10.2, at most about 10.1, at most about 10, at most about 9.9, at most about 9.8, at most about 9.7, at most about 9.6, at most about 9.5, at most about 9.4, at most about 9.3, at most about 9.2, at most about 9.1, at most about 9, or less. In some cases, the high pH can be about 9, about 9.1, about 9.2, about 9.3, about 9.4, about 9.5, about 9.6, about 9.7, about 9.8, about 9.9, about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 12.6, about 12.7, about 12.8, about 12.9, about 13, about 13.1, about 13.2, about 13.3, about 13.4, about 13.5, about 13.6, about 13.7, about 13.8, about 13.9, or about 14. A chemical group can release the native protein, non-native protein, non-native polypeptide, and/or non-native peptide from the support. The chemical group can be an acid and/or a base. The chemical group can be an alcohol. The chemical group can be pnictigen hydride, trifluoroacetic acid (TFA), tetrafluoroethylene (TFE), Copper acetate (CuOAc₂), pyridine, N,N-diisopropylethylamine (DIPEA), dimethylformamide (DMF), trimethylsilyldiazomethane (TMS-CHN₂), a thiol, p-nitro phenyl chloroformate (p-NO₂ PhOCOCI), hydrazine (NH₂NH₂), sodium borohydride (NABH₄), ethanol, dichloromethane (DCM), methanol, acetic acid, or any combination thereof.

In some cases, the native protein, non-native protein, non-native polypeptide, and/or non-native peptide may be released from the support (e.g., via cleavage). In some cases, the native protein, non-native protein, non-native polypeptide, and/or non-native peptide is released from the support by cleavage of a cleavable linker. The cleavable linker may be cleavable by an enzyme.

In some cases, the native protein, non-native protein, non-native polypeptide, and/or non-native peptide may be denatured subsequent to release from the support. The native protein, non-native protein, non-native polypeptide, and/or non-native peptide can be denatured by temperature, chemicals, detergents, and/or mechanical methods. In some cases, the native protein, non-native protein, non-native polypeptide, and/or non-native peptide can be denatured at a temperature from about 30° C. to about 40° C., from about 40° C. to about 50° C., or about 50° C. to about 60° C. In some cases, the native protein, non-native protein, non-native polypeptide, and/or non-native peptide can be denatured at a temperature of at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., or more. In some cases, the native protein, non-native protein, non-native polypeptide, and/or non-native peptide can be denatured at a temperature of at most about 60° C., at most about 59° C., at most about 58° C., at most about 57° C., at most about 56° C., at most about 55° C., at most about 54° C., at most about 53° C., at most about 52° C., at most about 51° C., at most about 50° C., at most about 49° C., at most about 48° C., at most about 47° C., at most about 46° C., at most about 45° C., at most about 44° C., at most about 43° C., at most about 42° C., at most about 41° C., at most about 40° C., at most about 39° C., at most about 38° C., at most about 37° C., at most about 36° C., at most about 35° C., at most about 34° C., at most about 33° C., at most about 32° C., at most about 31° C., at most about 30° C., or less. In some cases, the native protein, non-native protein, non-native polypeptide, and/or non-native peptide can be denatured at a temperature of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In some cases, the native protein, non-native protein, non-native polypeptide, and/or non-native peptide can be denatured chemically. In some cases, the native protein, non-native protein, non-native polypeptide, and/or non-native peptide can be denatured by an acid (e.g., trifluoroacetic acid, hydrochloric acid) and/or an alcohol (e.g., monohydric methanol, ethanol). In some cases, the native protein, non-native protein, non-native polypeptide, and/or non-native peptide can be denatured by a detergent (e.g., urea, guanidinium chloride). In some cases, the native protein, non-native protein, non-native polypeptide, and/or non-native peptide can be denatured mechanically (e.g., shaking).

In some cases, the native protein, non-native protein, non-native polypeptide, and/or non-native peptide may be digested into one or more peptide and/or one or more proteins (e.g., subsequent to releasing the native protein, non-native protein, non-native polypeptide, and/or non-native peptide from the support), for example, as depicted by in FIG. 4. In some cases, the capture reagent releases the native protein, non-native protein, non-native polypeptide, and/or non-native peptide through a hydrolysis (e.g., carboxyl-substituted amide). In some cases, the hydrolysis of the carboxyl-substituted amide can further result in condensation of the resulting diacid to reform the acid anhydride. In some cases, the acid anhydride can be a cyclic anhydride. In some cases, the cyclic anhydride can be a 2,5-furanedionyl moiety. In some cases, the 2,5-furanedionyl moiety can be a citraconic anhydride moiety. In some cases, the hydrolysis of the capture reagent results in the release of the native protein, non-native protein, non-native polypeptide, and/or non-native peptide from the support. In some cases, the hydrolysis can result in the reformation of the amine group on the native protein, non-native protein, non-native polypeptide, and/or non-native peptide. In some cases, the amine group can be reformed on a terminal amino acid residue and/or non-terminal amino acid residue. In some cases, the hydrolysis can result in the reformation of the capture reagent on the support. The native protein, non-native protein, non-native polypeptide, and/or non-native peptide can be digested by cleavage. In some cases, the cleavage may be an enzymatic cleavage. The enzymatic cleavage can comprise the use of a single protease, a series of protease (e.g., provided in a specific order), or a combination of proteases. In some cases, the enzymatic cleavage can be performed by one or more proteases. The one or more proteases can be a serine protease, a cysteine protease, a threonine protease, as aspartic protease, a glutamic protease, a metalloprotease, and/or as asparagine peptide lyase. Non-limiting examples of proteases and the one or more protease-associated cleavage sites in proteins are shown in TABLE 1. In some cases, the enzymatic cleavage is performed by an ArgC enzyme and/or a proalanase enzyme.

TABLE 1
Examples of Proteases

| Protease | Cleavage Site |
|---|---|
| Carboxypeptidase A | C-terminal exopeptidase |
| Carboxypeptidase B | C-terminal exopeptidase; specific for lysine |
| | or arginine |
| Carboxypeptidase P | C-terminal exopeptidase |
| Carboxypeptidase Y | C-terminal exopeptidase |
| Cathepsin C | N-terminal exopeptidase; removes N-terminal |
| | dipeptide (except when N-terminal amino acid |
| | is lysine or arginine, or when 2n^{d} or 3^{rd} amino |
| | acid from N-terminal is proline) |
| Chymotrypsin | C-terminal exopeptidase ; specific for |
| | phenylalanine, tryptophan, and tyrosine |
| Clostripain | Arginine |
| Elastase | Alanine, Valine, Serine, Glycine, Leucine, or |
| | Isoleucine |
| Endoproteinase Arg-C | Arginine |
| Endoproteinase Glu-C | Glutamic Acid |
| Endoproteinase Lys-C | Lysine |
| Glutamyl | Glutamic acid |
| endopeptidase | |
| Kallikrein (Plasma) | Lysine or Arginine |
| Papain | Lysine or Arginine followed by a hydrophobic |
| | residue |
| Pepsin | Leucine, Phenylalanine, Tryptophan or Tyrosine |
| Proteinase K | Aliphatic and aromatic amino acids |
| Subtilisin | Hydrophobic amino acids |
| TEV Protease | Specific for the sequences Glutamic acid- |
| | Asparagine-Leucine-Tyrosine-Phenylalanine- |
| | Glutamine-Glycine and Glutamic acid- |
| Thermolysin | Isoleucine, Methionine, Phenylalanine, |
| | Tryptophan, Tyrosine, or Valine |
| Trypsin | Lysine or Arginine |

In some cases, the cleavage may a chemical cleavage. The chemical cleavage can involve an inorganic compound. The inorganic compound can comprise cyanogen bromide, hydroxylamine, and/or hydrochloric acid (HCl). The chemical cleavage can involve an organic compound. The organic compound can comprise BNPS-skatole, formic acid, and/or 2-nitro-5-thiocyanobenzoic acid. In some cases, the chemical cleavage can occur at a temperature from 30° C. to about 60° C. In some cases, the chemical cleavage can occur at a temperature of at least about 30° C., at least about 35° C., at least about 40° C., at least about 45° C., at least about 50° C., at least about 55° C., at least about 60° C., or more. In some cases, the chemical cleavage can occur at a temperature of at most about 60° C., at most about 55° C., at most about 50° C., at most about 45° C., at most about 40° C., at most about 35° C., at most about 30° C., or less. In some cases, the chemical cleavage can occur at a temperature of about 30° C., about 35° C., about 40° C., about 45° C., about 50° C., about 55° C., or about 60° C. In some cases, the cleavage may be a combination (e.g., parallel or sequential use) of chemical and enzymatic cleavage reagents. In some cases, the cleavage may be a mechanical cleavage. A mechanical cleavage can involve sonication, liquid nitrogen grinding, bead beaters, and/or physical grinding (e.g., mortar and pestle).

In some cases, the method can comprise one or more cleavage operations. In some cases, the method can comprise from about one to about two cleavage operations, from about two to about three cleavage operations, from about three to about four cleavage operations, from about four to about five cleavage operations, from about five to about six cleavage operations, from about six cleavage operations to about seven cleavage operations, from about seven to about eight cleavage operations, from about eight to about nine cleavage operations, or from about nine to about ten cleavage operations. In some cases, the method can comprise at least about one cleavage operation, at least about two cleavage operations, at least about three cleavage operations, at least about four cleavage operations, at least about five cleavage operations, at least about six cleavage operations, at least about seven cleavage operations, at least about eight cleavage operations, at least about nine cleavage operations, at least about ten cleavage operations, or more. In some cases, the method can comprise at most about 10 cleavage operations, at most about nine cleavage operations, at most about eight cleavage operations, at most about seven cleavage operations, at most about six cleavage operations, at most about five cleavage operations, at most about four cleavage operations, at most about three cleavage operations, at most about two cleavage operations, at most about one cleavage operation, or less. In some cases, the method can comprise about one cleavage operation, about two cleavage operations, about three cleavage operations, about four cleavage operations, about five cleavage operations, about six cleavage operations, about seven cleavage operations, about eight cleavage operations, about nine cleavage operations, or about ten cleavage operations.

Protein cleavage conditions may be achieved with a solvent. The solvent may be an aqueous solvent, an organic solvent, or a combination or mixture thereof. The solvent may be an organic solvent. The organic solvent may comprise a miscibility with water. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or a polar protic solvent (e.g., isopropanol (IPA), ethanol, methanol, acetic acid, water, etc.). The solvent may be DMF. The solvent may be a C₁-C₁₂ haloalkane. The C₁-C₁₂ haloalkane may be DCM. The solvent may be a mixture of two or more solvents. The mixture of two or more solvents may be a mixture of a polar aprotic solvent and a C₁-C₁₂ haloalkane. The mixture of two or more solvents may be a mixture of DMF and DCM. The mixture of solvents may be any combination thereof.

The native protein, non-native protein, non-native polypeptide, and/or non-native peptide can be digested to form one or more peptides and/or one or more proteins. Each of the one or more peptides and/or one or more proteins may comprise from about 5 amino acids to about 200 amino acids. In some cases, the one or more peptides and/or one or more proteins may be from about 5 amino acids to about 20 amino acids, from about 5 amino acids to about 50 amino acids, from about 5 amino acids to about 5 amino acids to about 100 amino acids, from about 5 amino acids to about 150 amino acids, or from about 5 amino acids to about 200 amino acids. In some cases, the one or more peptides and/or one or more proteins are at least about 5 amino acids, at least about 10 amino acids, at least about 15 amino acids, at least about 20 amino acids, at least about 25 amino acids, at least about 30 amino acids, at least about 35 amino acids, at least about 40 amino acids, at least about 50 amino acids, at least about 75 amino acids, at least about 100 amino acids, at least about 125 amino acids, at least about 150 amino acids, at least about 175 amino acids, at least about 200 amino acids or more.

In some cases, each of the one or more peptides and/or one or more proteins may be at most about 200 amino acids, at most about 175 amino acids, at most about 150 amino acids, at most about 125 amino acids, at most about 100 amino acids, at most about 75 amino acids, at most about 50 amino acids, at most about 45 amino acids, at most about 40 amino acids, at most about 35 amino acids, at most about 30 amino acids, at most about 25 amino acids, at most about 20 amino acids, at most about 15 amino acids, at most about 10 amino acids, at most about 5 amino acids, or less.

In some cases, each of the one or more peptides and/or one or more proteins may be about 5 amino acids, about 10 amino acids, about 15 amino acids, about 20 amino acids, about 25 amino acids, about 30 amino acids, about 35 amino acids, about 40 amino acids, about 45 amino acids, about 50 amino acids, about 75 amino acids, about 100 amino acids, about 125 amino acids, about 150 amino acids, about 175 amino acids, or about 200 amino acids.

In some cases, the native protein, non-native protein, non-native polypeptide, and/or non-native peptide is digested to form from about two peptides and/or proteins to about 10,000 peptides and/or proteins. In some cases, the native protein, non-native protein, non-native polypeptide, and/or non-native peptide is digested to form from about two peptides and/or proteins to about 50 peptides and/or proteins, from about 50 peptides and/or proteins to about 100 peptides and/or proteins, from about 100 peptides and/or proteins to about 200 peptides and/or proteins, from about 200 peptides and/or proteins to about 300 peptides and/or proteins, from about 300 peptides and/or proteins to about 400 peptides and/or proteins, from about 400 peptides and/or proteins to about 500 peptides and/or proteins, from about 500 peptides and/or proteins to about 600 peptides and/or proteins, from about 600 peptides and/or proteins to about 700 peptides and/or proteins, from about 700 peptides and/or proteins to about 800 peptides and/or proteins, from about 800 peptides and/or proteins to about 900 peptides and/or proteins, from about 900 peptides and/or proteins to about 1,000 peptides and/or proteins, from about 1,000 peptides and/or proteins to about 2,000 peptides and/or proteins, from about 2,000 peptides and/or proteins to about 3,000 peptides and/or proteins, from about 3,000 peptides and/or proteins to about 4,000 peptides and/or proteins, from about 4,000 peptides and/or proteins to about 5,000 peptides and/or proteins, from about 5,000 peptides and/or proteins to about 6,000 peptides and/or proteins, from about 6,000 peptides and/or proteins to about 7,000 peptides and/or proteins, from about 7,000 peptides and/or proteins to about 8,000 peptides and/or proteins, from about 8,000 peptides and/or proteins to about 9,000 peptides and/or proteins, or from about 9,000 peptides and/or proteins to about 10,000 peptides and/or proteins. In some cases, the native protein, non-native protein, non-native polypeptide, and/or non-native peptide is digested to form at least about two peptides and/or proteins, at least about ten peptides and/or proteins, at least about 20 peptides and/or proteins, at least about 30 peptides and/or proteins, at least about 40 peptides and/or proteins, at least about 50 peptides and/or proteins, at least about 60 peptides and/or proteins, at least about 70 peptides and/or proteins, at least about 80 peptides and/or proteins, at least about 90 peptides and/or proteins, at least about 100 peptides and/or proteins, at least about 150 peptides and/or proteins, at least about 200 peptides and/or proteins, at least about 250 peptides and/or proteins, at least about 300 peptides and/or proteins, at least about 350 peptides and/or proteins, at least about 400 peptides and/or proteins, at least about 450 peptides and/or proteins, at least about 500 peptides and/or proteins, at least about 550 peptides and/or proteins, at least about 600 peptides and/or proteins, at least about 650 peptides and/or proteins, at least about 700 peptides and/or proteins, at least about 750 peptides and/or proteins, at least about 800 peptide and/or proteins, at least about 850 peptides and/or proteins, at least about 900 peptides and/or proteins, at least about 950 peptides and/or proteins, at least about 1,000 peptides and/or proteins, at least about 1,100 peptides and/or proteins, at least about 1,200 peptides and/or proteins, at least about 1,300 peptides and/or proteins, at least about 1,400 peptides and/or proteins, at least about 1,500 peptides and/or proteins, at least about 1,600 peptides and/or proteins, at least about 1,700 peptides and/or proteins, at least about 1,800 peptides and/or proteins, at least about 1,900 peptides and/or proteins, at least about 2,000 peptides and/or proteins, at least about 2,100 peptides and/or proteins, at least about 2,200 peptides and/or proteins, at least about 2,300 peptides and/or proteins, at least about 2,400 peptides and/or proteins, at least about 2,500 peptides and/or proteins, at least about 2,600 peptides and/or proteins, at least about 2,700 peptides and/or proteins, at least about 2,800 peptides and/or proteins, at least about 2,900 peptides and/or proteins, at least about 3,000 peptides and/or proteins, at least about 3,100 peptides and/or proteins, at least about 3,200 peptides and/or proteins, at least about 3,300 peptides and/or proteins, at least about 3,400 peptides and/or proteins, at least about 3,500 peptides and/or proteins, at least about 3,600 peptides and/or proteins, at least about 3,700 peptides and/or proteins, at least about 3,800 peptides and/or proteins, at least about 3,900 peptides and/or proteins, at least about 4,000 peptides and/or proteins, at least about 4,200 peptides and/or proteins, at least about 4,300 peptides and/or proteins, at least about 4,400 peptides and/or proteins, at least about 4,500 peptides and/or proteins, at least about 4,600 peptides and/or proteins, at least about 4,700 peptides and/or proteins, at least about 4,800 peptides and/or proteins, at least about 4,900 peptides and/or proteins, at least about 5,000 peptides and/or proteins, at least about 5,100 peptides and/or proteins, at least about 5,200 peptides and/or proteins, at least about 5,300 peptides and/or proteins, at least about 5,400 peptides and/or proteins, at least about 5,500 peptides and/or proteins, at least about 5,600 peptides and/or proteins, at least about 5,700 peptides and/or proteins, at least about 5,800 peptides and/or proteins, at least about 5,900 peptides and/or proteins, at least about 6,000 peptides and/or proteins, at least about 6,100 peptides and/or proteins, at least about 6,200 peptides and/or proteins, at least about 6,300 peptides and/or proteins, at least about 6,400 peptides and/or proteins, at least about 6,500 peptides and/or proteins, at least about 6,600 peptides and/or proteins, at least about 6,700 peptides and/or proteins, at least about 6,800 peptides and/or proteins, at least about 6,900 peptides and/or proteins, at least about 7,000 peptides and/or proteins, at least about 7,100 peptides and/or proteins, at least about 7,200 peptides and/or proteins, at least about 7,300 peptides and/or proteins, at least about 7,400 peptides and/or proteins, at least about 7,500 peptides and/or proteins, at least about 7,600 peptides and/or proteins, at least about 7,700 peptides and/or proteins, at least about 7,800 peptides and/or proteins, at least about 7,900 peptides and/or proteins, at least about 8,000 peptides and/or proteins, at least about 8,100 peptides and/or proteins, at least about 8,200 peptides and/or proteins, at least about 8,300 peptides and/or proteins, at least about 8,400 peptides and/or proteins, at least about 8,500 peptides and/or proteins, at least about 8,600 peptides and/or proteins, at least about 8,700 peptides and/or proteins, at least about 8,800 peptides and/or proteins, at least about 8,900 peptides and/or proteins, at least about 9,000 peptides and/or proteins, at least about 9,100 peptides and/or proteins, at least about 9,200 peptides and/or proteins, at least about 9,300 peptides and/or proteins, at least about 9,400 peptides and/or proteins, at least about 9,500 peptides and/or proteins, at least about 9,600 peptides and/or proteins, at least about 9,700 peptides and/or proteins, at least about 9,800 peptides and/or proteins, at least about 9,900 peptides and/or proteins, at least about 10,000 peptides and/or proteins, or more. In some cases, the native protein, non-native protein, non-native polypeptide, and/or non-native peptide is digested to form at most about 10,000 peptides and/or proteins, at most about 9,900 peptides and/or proteins, at most about 9,800 peptides and/or proteins, at most about 9,700 peptides and/or proteins, at most about 9,600 peptides and/or proteins, at most about 9,500 peptides and/or proteins, at most about 9,400 peptides and/or proteins, at most about 9,300 peptides and/or proteins, at most about 9,200 peptides and/or proteins, at most about 9,100 peptides and/or proteins, at most about 9,000 peptides and/or proteins, at most about 8,900 peptides and/or proteins, at most about 8,800 peptides and/or proteins, at most about 8,700 peptides and/or proteins, at most about 8,600 peptides and/or proteins, at most about 8,500 peptides and/or proteins, at most about 8,400 peptides and/or proteins, at most about 8,300 peptides and/or proteins, at most about 8,200 peptides and/or proteins, at most about 8,100 peptides and/or proteins, at most about 8,000 peptides and/or proteins, at most about 7,900 peptides and/or proteins, at most about 7,800 peptides and/or proteins, at most about 7,700 peptides and/or proteins, at most about 7,600 peptides and/or proteins, at most about 7,500 peptides and/or proteins, at most about 7,400 peptides and/or proteins, at most about 7,300 peptides and/or proteins, at most about 7,200 peptides and/or proteins, at most about 7,100 peptides and/or proteins, at most about 7,000 peptides and/or proteins, at most about 6,900 peptides and/or proteins, at most about 6,800 peptides and/or proteins, at most about 6,700 peptides and/or proteins, at most about 6,600 peptides and/or proteins, at most about 6,500 peptides and/or proteins, at most about 6,400 peptides and/or proteins, at most about 6,300 peptides and/or proteins, at most about 6,200 peptides and/or proteins, at most about 6,100 peptides and/or proteins, at most about 6,000 peptides and/or proteins, at most about 5,900 peptides and/or proteins, at most about 5,800 peptides and/or proteins, at most about 5,700 peptides and/or proteins, at most about 5,600 peptides and/or proteins, at most about 5,500 peptides and/or proteins, at most about 5,400 peptides and/or proteins, at most about 5,300 peptides and/or proteins, at most about 5,200 peptides and/or proteins, at most about 5,100 peptides and/or proteins, at most about 5,000 peptides and/or proteins, at most about 4,900 peptides and/or proteins, at most about 4,800 peptides and/or proteins, at most about 4,700 peptides and/or proteins, at most about 4,600 peptides and/or proteins, at most about 4,500 peptides and/or proteins, at most about 4,400 peptides and/or proteins, at most about 4,300 peptides and/or proteins, at most about 4,200 peptides and/or proteins, at most about 4,100 peptides and/or proteins, at most about 4,000 peptides and/or proteins, at most about 3,900 peptides and/or proteins, at most about 3,800 peptides and/or proteins, at most about 3,700 peptides and/or proteins, at most about 3,600 peptides and/or proteins, at most about 3,500 peptides and/or proteins, at most about 3,400 peptides and/or proteins, at most about 3,300 peptides and/or proteins, at most about 3,200 peptides and/or proteins, at most about 3,100 peptides and/or proteins, at most about 3,000 peptides and/or proteins, at most about 2,900 peptides and/or proteins, at most about 2,800 peptides and/or proteins, at most about 2,700 peptides and/or proteins, at most about 2,600 peptides and/or proteins, at most about 2,500 peptides and/or proteins, at most about 2,400 peptides and/or proteins, at most about 2,300 peptides and/or proteins, at most about 2,200 peptides and/or proteins, at most about 2,100 peptides and/or proteins, at most about 2,000 peptides and/or proteins, at most about 1,900 peptides and/or proteins, at most about 1,800 peptides and/or proteins, at most about 1,700 peptides and/or proteins, at most about 1,600 peptides and/or proteins, at most about 1,500 peptides and/or proteins, at most about 1,400 peptides and/or proteins, at most about 1,300 peptides and/or proteins, at most about 1,200 peptides and/or proteins, at most about 1,100 peptides and/or proteins, at most about 1,000 peptides and/or proteins, at most about 950 peptides and/or proteins, at most about 900 peptides and/or proteins, at most about 850 peptides and/or proteins, at most about 800 peptides and/or proteins, at most about 750 peptides and/or proteins, at most about 700 peptides and/or proteins, at most about 650 peptides and/or proteins, at most about 600 peptides and/or proteins, at most about 550 peptides and/or proteins, at most about 500 peptides and/or proteins, at most about 450 peptides and/or proteins, at most about 400 peptides and/or proteins, at most about 350 peptides and/or proteins, at most about 300 peptides and/or proteins, at most about 250 peptides and/or proteins, at most about 200 peptides and/or proteins, at most about 150 peptides and/or proteins, at most about 100 peptides and/or proteins, at most about 90 peptides and/or proteins, at most about 80 peptides and/or proteins, at most about 70 peptides and/or proteins, at most about 60 peptides and/or proteins, at most about 50 peptides and/or proteins, at most about 40 peptides and/or proteins, at most about 30 peptides and/or proteins, at most about 20 peptides and/or proteins, at most about 10 peptides and/or proteins, at most about 2 peptides and/or proteins, or less. In some cases, the native protein, non-native protein, non-native polypeptide, and/or non-native peptide is digested to form about two peptides and/or proteins, about 10 peptides and/or proteins, about 20 peptides and/or proteins, about 30 peptides and/or proteins, about 40 peptides and/or proteins, about 50 peptides and/or proteins, about 60 peptides and/or proteins, about 70 peptides and/or proteins, about 80 peptides and/or proteins, about 90 peptides and/or proteins, about 100 peptides and/or proteins, about 150 peptides and/or proteins, about 200 peptides and/or proteins, about 250 peptides and/or proteins, about 300 peptides and/or proteins, about 250 peptides and/or proteins, about 300 peptides and/or proteins, about 350 peptides and/or proteins, about 400 peptides and/or proteins, about 450 peptides and/or proteins, about 500 peptides and/or proteins, about 550 peptides and/or proteins, about 600 peptides and/or proteins, about 650 peptides and/or proteins, about 700 peptides and/or proteins, about 750 peptides and/or proteins, about 800 peptides and/or proteins, about 850 peptides and/or proteins, about 900 peptides and/or proteins, about 950 peptides and/or proteins, about 1,000 peptides and/or proteins, about 1,100 peptides and/or proteins, about 1,200 peptides and/or proteins, about 1,300 peptides and/or proteins, about 1,400 peptides and/or proteins, about 1,500 peptides and/or proteins, about 1,600 peptides and/or proteins, about 1,700 peptides and/or proteins, about 1,800 peptides and/or proteins, about 1,900 peptides and/or proteins, about 2,000 peptides and/or proteins, about 2,100 peptides and/or proteins, about 2,200 peptides and/or proteins, about 2,300 peptides and/or proteins, about 2,400 peptides and/or proteins, about 2,500 peptides and/or proteins, about 2,600 peptides and/or proteins, about 2,700 peptides and/or proteins, about 2,800 peptides and/or proteins, about 2,900 peptides and/or proteins, about 3,000 peptides and/or proteins, about 3,100 peptides and/or proteins, about 3,200 peptides and/or proteins, about 3,300 peptides and/or proteins, about 3,400 peptides and/or proteins, about 3,500 peptides and/or proteins, about 3,600 peptides and/or proteins, about 3,700 peptides and/or proteins, about 3,800 peptides and/or proteins, about 3,900 peptides and/or proteins, about 4,000 peptides and/or proteins, about 4,100 peptides and/or proteins, about 4,200 peptides and/or proteins, about 4,300 peptides and/or proteins, about 4,400 peptides and/or proteins, about 4,500 peptides and/or proteins, about 4,600 peptides and/or proteins, about 4,700 peptides and/or proteins, about 4,800 peptides and/or proteins, about 4,900 peptides and/or proteins, about 5,000 peptides and/or proteins, about 5,100 peptides and/or proteins, about 5,200 peptides and/or proteins, about 5,300 peptides and/or proteins, about 5,400 peptides and/or proteins, about 5,500 peptides and/or proteins, about 5,600 peptides and/or proteins, about 5,700 peptides and/or proteins, about 5,800 peptides and/or proteins, about 5,900 peptides and/or proteins, about 6,000 peptides and/or proteins, about 6,100 peptides and/or proteins, about 6,200 peptides and/or proteins, about 6,300 peptides and/or proteins, about 6,400 peptides and/or proteins, about 6,500 peptides and/or proteins, about 6,600 peptides and/or proteins, about 6,700 peptides and/or proteins, about 6,800 peptides and/or proteins, about 6,900 peptides and/or proteins, about 7,000 peptides and/or proteins, about 7,100 peptides and/or proteins, about 7,200 peptides and/or proteins, about 7,300 peptides and/or proteins, about 7,400 peptides and/or proteins, about 7,500 peptides and/or proteins, about 7,600 peptides and/or proteins, about 7,700 peptides and/or proteins, about 7,800 peptides and/or proteins, about 7,900 peptides and/or proteins, about 8,000 peptides and/or proteins, about 8,100 peptides and/or proteins, about 8,200 peptides and/or proteins, about 8,300 peptides and/or proteins, about 8,400 peptides and/or proteins, about 8,500 peptides and/or proteins, about 8,600 peptides and/or proteins, about 8,700 peptides and/or proteins, about 8,800 peptides and/or proteins, about 8,900 peptides and/or proteins, about 9,000 peptides and/or proteins, about 9,100 peptides and/or proteins, about 9,200 peptides and/or proteins, about 9,300 peptides and/or proteins, about 9,400 peptides and/or proteins, about 9,500 peptides and/or proteins, about 9,600 peptides and/or proteins, about 9,700 peptides and/or proteins, about 9,800 peptides and/or proteins, about 9,900 peptides and/or proteins, or about 10,000 peptides and/or proteins.

In some cases, the one or more labels can be coupled to the one or more external amino acids on the native protein, non-native protein, non-native polypeptide, and/or non-native peptide subsequent to digesting the native protein, non-native protein, non-native polypeptide, and/or non-native peptide. In some cases, the first reactive group may be coupled to the one or more external amino acids on the native protein, non-native protein, non-native polypeptide, and/or non-native peptide prior to digesting the native protein, non-native protein, non-native polypeptide, and/or non-native peptide. In some cases, the second reactive group may be coupled to the one or more internal amino acids on the one or more peptides and/or the one or more proteins prior to the coupling of the one or more additional labels. In some cases, the first reactive group and/or the second reactive group can be Click molecules.

The methods provided herein may comprise coupling the one or more peptides, one or more proteins, and/or the native protein, non-native protein, non-native polypeptide, and/or non-native peptide to an additional support, for example, as depicted by [406] in FIG. 4. The one or more peptides and/or one or more proteins can be directly coupled to the additional support through, such as, for example, a covalent bond (e.g., a peptide bond). The one or peptides and/or the one or more proteins can be directly coupled to the additional support through, such as, for example, a non-covalent bond (e.g., hydrophobic interactions, hydrogen bonds, electrostatic interactions, van der Waal interactions). The one or more peptides and/or the one or more proteins can be indirectly coupled to the additional support through, such as, for example, a spacer. In some cases, the additional support is different from the support. In some cases, the additional support is the same as the support.

In some cases, the one or more peptides and/or the one or more proteins can be released from the additional support. In some cases, the one or more peptides and/or the one or more protein can be digested into another one or more peptides and/or another one or more proteins. The another one or more peptides and/or the another one or more proteins can be coupled to another additional support subsequent to the digestion of the one or more peptides and/or the one or more proteins. The another additional support can be the same as the support and/or the additional support. The another additional support can be different from the support and/or the additional support. In some cases, the process of releasing the one or more peptides and/or one or more proteins from an additional support, digesting the one or more peptides and/or one or more proteins, and/or coupling to another additional support can be repeated from about one time to about ten times. In some examples, the process can be repeated from about one time to about two times, from about two times to about three times, from about three times to about four times, from about four times to about five times, from about five times to about six times, from about six times to about seven times, from about seven times to about eight times, from about eight times to about nine times, or from about nine times to about ten times. In some examples, the process can be repeated at least about one time, at least about two times, at least about three times, at least about four times, at least about five times, at least about six times, at least about seven times, at least about eight times, at least about nine times, at least about ten times, or more. In some examples, the process can be repeated at most about ten times, at most about nine times, at most about eight times, at most about seven times, at most about six times, at most about five times, at most about four times, at most about three times, at most about two times, at most about one time, or less. In some examples, the process can be repeated about one time, about two times, about three times, about four times, about five times, about six times, about seven times, about eight times, about nine times, or about ten times.

In some cases, the process can be repeated until the one or more peptides and/or one or more proteins are a length from about 5 amino acids to about 200 amino acids. In some cases, the process can be repeated until the one or more peptides and/or one or more proteins are a length from about 5 amino acids to about 10 amino acids, from about 10 amino acids to about 20 amino acids, from about 20 amino acids to about 30 amino acids, from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 100 amino acids, from about 100 amino acids to about 110 amino acids, from about 110 amino acids to about 120 amino acids, from about 120 amino acids to about 130 amino acids, from about 130 amino acids to about 140 amino acids, from about 140 amino acids to about 150 amino acids, from about 150 amino acids to about 160 amino acids, from about 160 amino acids to about 170 amino acids, from about 170 amino acids to about 180 amino acids, from about 180 amino acids to about 190 amino acids, or from about 190 amino acids to about 200 amino acids. In some cases, the process can be repeated until the one or more peptides and/or one or more proteins are a length of at least about 5 amino acids, at least about 10 amino acids, at least about 15 amino acids, at least about 20 amino acids, at least about 25 amino acids, at least about 30 amino acids, at least about 35 amino acids, at least about 40 amino acids, at least about 45 amino acids, at least about 50 amino acids, at least about 55 amino acids, at least about 60 amino acids, at least about 65 amino acids, at least about 70 amino acids, at least about 75 amino acids, at least about 80 amino acids, at least about 85 amino acids, at least about 90 amino acids, at least about 95 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, or more. In some cases, the process can be repeated until the one or more peptides and/or one or more proteins are a length of at most about 200 amino acids, at most about 190 amino acids, at most about 180 amino acids, at most about 170 amino acids, at most about 160 amino acids, at most about 150 amino acids, at most about 140 amino acids, at most about 130 amino acids, at most about 120 amino acids, at most about 110 amino acids, at most about 100 amino acids, at most about 95 amino acids, at most about 90 amino acids, at most about 85 amino acids, at most about 80 amino acids, at most about 75 amino acids, at most about 70 amino acids, at most about 65 amino acids, at most about 60 amino acids, at most about 55 amino acids, at most about 50 amino acids, at most about 45 amino acids, at most about 40 amino acids, at most about 35 amino acids, at most about 30 amino acids, at most about 25 amino acids, at most about 20 amino acids, at most about 15 amino acids, at most about 10 amino acids, at most about 5 amino acids, or less. In some cases, the process can be repeated until the one or more peptides and/or one or more proteins are a length of about 5 amino acids, about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 110 amino acids, about 120 amino acids, about 130 amino acids, about 140 amino acids, about 150 amino acids, about 160 amino acids, about 170 amino acids, about 180 amino acids, about 190 amino acids, or about 200 amino acids.

The methods provided herein may comprise coupling one or more additional labels to the one or more internal amino acids on the one or more peptides and/or the one or more proteins. For example, as depicted by [407] in FIG. 4, the one or more additional labels can be coupled to the one or more internal amino acids on the one or more peptides and/or one or more proteins. In some cases, the one or more internal amino acids on the native protein, non-native protein, non-native polypeptide, and/or non-native peptide may be solvent accessible on the one or more peptides and/or the one or more proteins. In some cases, the one or more internal amino acids may be coupled to one or more additional labels prior to coupling the one or more peptides and/or the one or more proteins to an additional support. The additional support can comprise the support that couples to the native protein, non-native protein, non-native polypeptide, and/or non-native peptide. The additional support can comprise a different support. In some cases, the one or more internal amino acids on the one or more peptides and/or the one or more proteins may be coupled to one or more additional labels subsequent to coupling the one or more peptides and/or the one or more proteins to the additional support.

The one or more additional labels may be directly coupled to the side group of the one or more internal amino acids on the one or more peptides and/or the one or more proteins, such as, for example, through a covalent bond (e.g., a peptide bond). The one or more additional labels may be directly coupled to the side group of the one or more internal amino acids on the one or more peptides and/or the one or more proteins, such as, for example through a non-covalent bond (e.g., an electrostatic interaction, a hydrogen bond, a van der Waals interaction, and/or a hydrophobic interaction). In some cases, the one or more internal amino acids can comprise terminal amino acid residues and/or non-terminal amino acids residues. In some cases, the one or more internal amino acid residues can comprise a terminal amino acid. In some cases, the one or more internal amino acid can comprise the N-terminal amino acid residue and/or the C-terminal amino acid residue. In some cases, the one or more internal amino acid residues can comprise non-terminal amino acid residues. In some cases, the one or more internal amino acid residues can comprise terminal amino acids and/or non-terminal amino acids. In some cases, the one or more internal amino acids can comprise the N-terminal amino acid residue. In some cases, the one or more internal amino acids can comprise the C-terminal amino acid residue. In some cases, the one or more internal amino acids can comprise non-terminal amino acids. In some cases, the one or more additional labels may not alter the native structure of the native protein, non-native protein, non-native polypeptide, and/or non-native peptides when coupled to the one or more internal amino acids.

The one or more additional labels may be coupled to a terminal end of the native protein, non-native protein, non-native polypeptide, and/or non-native peptide. The terminal end can comprise the N-terminal amino acid residue and/or the C-terminal amino acid residue. The one or more additional labels may be coupled to a non-terminal amino acid residue on the one or more peptides and/or one or more proteins. The non-terminal amino acid can comprise any residue on the one or more peptides and/or one or more proteins excluding the N-terminal residue and/or the C-terminal residue. In some cases, the one or more additional labels may be coupled to a terminal amino acid residue and/or a non-terminal amino acid residue. The one or more additional labels may be irreversibly or reversibly coupled to the side group of the one or more internal amino acids on the one or more peptides and/or the one or more proteins. The one or more additional labels may be indirectly coupled to the one or more internal amino acids on the one or more peptides and/or the one or more proteins. The side group of the one or more internal amino acids on the one or more peptides and/or the one or more proteins may be coupled to a first reactive group (e.g., Click). The first reactive group can be directly coupled to the side group of the one or more internal amino acids on the one or more peptides and/or the one or more proteins, such as, for example, through a covalent bond (e.g., a peptide bond). The first reactive group can be directly coupled to the side group of the one or more internal amino acids on the one or more peptides and/or the one or more proteins, such as, for example, through a non-covalent bond (e.g., an electrostatic interaction, a hydrogen bond, a van der Waals interaction, and/or a hydrophobic interaction). The first reactive group may be irreversibly or reversibly coupled to the side group of the one or more internal amino acids on the one or more peptides and/or the one or more proteins. In some cases, the first reactive group is coupled to the one or more additional labels (e.g., Clack, a second reactive group). The first reactive group may be directly coupled to the one or more additional labels, such as, for example, through a covalent bond (e.g., peptide bond). The first reactive group may be directly coupled to the one or more additional labels, such as, for example, through a non-covalent bond (e.g., an electrostatic interaction, a hydrogen bond, a van der Waals interaction, and/or a hydrophobic interaction. The first reactive group can be irreversibly or reversibly coupled to the one or more additional labels.

In some cases, the one or more additional labels can couple to acidic amino acid residues (e.g., aspartic acid and/or glutamic acid), histidine amino acid residues, and/or tyrosine amino acid residues.

In some cases, the one or more peptides and/or the one or more proteins can be released from the additional support, for example, as depicted by in FIG. 4. The one or more peptides and/or the one or more proteins can be released from the additional support using pH, chemical, and/or enzymatic methods as disclosed herein.

In some cases, the one or more labels coupled to one or more external amino acids on the native protein, non-native protein, non-native polypeptide, and/or non-native peptide and/or the one or more additional labels coupled to the one or more internal amino acids on the one or more peptides and/or the one or more proteins can be detected. The one or more labels and/or one or more additional labels can be detected by fluorescent microscope. The one or more labels coupled to the one or more external amino acids on the native protein, non-native protein, non-native polypeptide, and/or non-native peptide and/or the one or more additional labels coupled to the one or more internal amino acids on the one or more peptides and/or the one or more proteins can be detected by fluorosequencing, for example, as depicted by [409] in FIG. 4. In some cases, the fluorosequencing can detect a pattern of positions of the one or more labels and/or a pattern of positions of the one or more additional labels. In some cases, the one or more labels and/or the one or more additional labels may be detected by a protein sequencing method (e.g., Edman degradation, mass spectrometry). The one or more labels and/or the one or more additional labels can be used to determine the sequence of protein and/or peptide and/or the one or more proteins, for example, as depicted by [412] in FIG. 4. The one or more labels and/or the one or more additional labels can be used to determine which amino acid residues are external amino acid residues and/or internal amino acids residues, for example, as depicted by [411] in FIG. 4. In some embodiments, the detecting the one or more external amino acids on the native protein, non-native protein, non-native polypeptide, and/or non-native peptide to identify or determine the native structure of the native protein, non-native protein, non-native polypeptide, and/or non-native peptide using the one or more labels may be subsequent to the providing a native protein, non-native protein, non-native polypeptide, and/or non-native peptide coupled to a support.

In some cases, the one or more labels and/or the one or more additional labels can be used to determine the native structure of the native protein, non-native protein, non-native polypeptide, and/or non-native peptide, for example, as depicted by [410] in FIG. 4. Mapping the pattern of the one or more external amino acids on the native protein, non-native protein, non-native polypeptide, and/or non-native peptide and/or the pattern of the one or more internal amino acids to a reference protein sequence can infer residues or stretches of the native protein, non-native protein, non-native polypeptide, and/or non-native peptide that has buried amino acid residues and/or surface-exposed amino acid residues. The reference protein sequence may be from a pathogen. Non-limiting examples of pathogens include bacteria, viruses, fungi, parasites, and helminths. The reference protein sequence may be from lab strain of a bacteria, virus, and/or yeast. The reference protein sequence may be from yeast. The reference sequence can be from an invertebrate. The reference sequence can be from a vertebrate. The reference sequence can be from a mammal (e.g., mouse, rat, rabbit, non-human primate, human). The reference sequence can be from a subject. The reference sequence can be from a subject taken at an earlier time point.

The native structure of the native protein, non-native protein, non-native polypeptide, and/or non-native peptide can be determined using a partial sequence of the native protein, non-native protein, non-native polypeptide, and/or non-native peptide. In some cases, the native structure of the native protein, non-native protein, non-native polypeptide, and/or non-native peptide can be generated using a combination of a partial sequence of the native protein, non-native protein, non-native polypeptide, and/or non-native peptide and a database of folded protein structure (e.g., pdb database). In some cases, the partial sequence of the native protein, non-native protein, non-native polypeptide, and/or non-native peptide can be generated using any of the methods disclosed herein. In some cases, the partial sequence of the native protein, non-native protein, non-native polypeptide, and/or non-native peptide can be generated using fluorosequencing. The native structure of the native protein, non-native protein, non-native polypeptide, and/or non-native peptide can be generated computationally. In some cases, the native structure of the native protein, non-native protein, non-native polypeptide, and/or non-native peptide can be generated using a hidden Markov model and/or a protein structure database. In some cases, the hidden Markov model and/or protein structure database can predict the probability of a labeled amino acid residue being an external amino acid residue and/or an internal amino acid residue.

In some cases, the native structure of the native protein, non-native protein, non-native polypeptide, and/or non-native peptide can be detected while native protein, non-native protein, non-native polypeptide, and/or non-native peptide is coupled to the surface. In some cases, the native structure of the native protein, non-native protein, non-native polypeptide, and/or non-native peptide may be detected while the one or more peptides and/or the one or more proteins are coupled to the additional surface.

Labels with Multiple Reactive Groups

A two-operation labeling chemistry (e.g., click-clack) can be used to label the one or more external amino acids and/or the one or more internal amino acids. The one or more external amino acids and/or the one or more internal amino acids can be labeled first through a first reactive group (e.g., bi-functional small-molecule reagent, amino acid labeling moiety, click moiety, click). The first reactive group may be coupled with a second reactive group (e.g., functionalized fluorophore, fluorophore, clack moiety, clack).

Aspects of the present disclosure provide amino acid labels comprising a first reactive group for coupling to an amino acid of a protein and/or peptide (or a portion thereof, such as a reactive functional group of an amino acid side chain) and/or a second reactive group for coupling to a reporter moiety or a protecting group. Such a system may be referred to as a “click-clack” labeling system, wherein a “click” reagent refers to a label configured to couple to an amino acid, and a “clack” reagent refers to a reporter moiety or protecting group configured to couple to the “click” reagent. The second reactive group of a label may be configured to reversibly or irreversibly couple to a reporter moiety, a protecting group, or any combination thereof. The second reactive group may be reversibly coupled to a protecting group, decoupled from the protecting group, and/or then coupled to a reporter moiety. For example, the label may be provided with a protecting group coupled to its first or second reactive group (e.g., a diol coupled to an aldehyde reactive group of the label). Such a modular labeling process may enable multi-amino acid labeling schemes with diminished cross-reactivity between amino acid and/or label types. Such a labeling process may also enable the use of chemically sensitive reporter moieties (e.g., pH sensitive or chemically quenchable dyes), by allowing their attachment following amino acid labeling operations. For example, a method may comprise selectively labeling cysteine residues of a peptide and/or a protein with a first label, selectively labeling lysine residues of the peptide and/or protein with a second label, selectively labeling carboxylate-containing residues (e.g., aspartate and/or glutamate) of the peptide and/or the protein with a third label, selectively labeling arginine residues of the peptide and/or protein with a fourth label, chemically modifying (e.g., oxidizing) methionine residues of the peptide and/or protein, selectively labeling the chemically modified methionine residues of the peptide and/or protein with a fifth label, and coupling different reporter moieties (e.g., different color dyes) to each of the first, second, third, fourth, and fifth labels in a single operation (e.g., upon addition of all labeling reagents simultaneously). It is also conceivable that one or more reporter fluorophores may directly label the amino acids on the protein and/or peptide. A bifunctional label of the present disclosure may prevent cross-reactivity between a first reactive group of a label and a reporter moiety. For example, the use of bifunctional labels may permit use of reporter moieties which are cross-reactive with a first reactive group of a label, such as an iodoacetamide-reactive dye and a label comprising a cysteine reactive iodoacetamide group. In some cases, the first reactive group is covalently coupled to the amino acid. In some cases, the second reactive group is covalently coupled the reporter moiety, the protecting group, or any combination thereof.

A label of the present disclosure may be used to crosslink two biological species, such as two amino acid residues. For example, a method may comprise coupling a lysine selective label to a first peptide and/or protein and a cysteine selective label to a second peptide and/or protein, and then cross-linking the lysine and cysteine selective labels. The cross-linking may directly couple (e.g., through a chemical bond) the lysine and cysteine selective labels, or may comprise a linker, such as a “clack” reagent configured to couple to second reactive groups on the lysine and cysteine selective labels.

Examples of amino acid selective labels comprising second reactive groups, as well as example reagent pairs for their syntheses, are provided in TABLE 2. A cysteine- and lysine-selective “Click” label may comprise an iodoacetamide as a first reactive group (e.g., for coupling to cysteine or lysine) and an azide as a second reactive group (e.g., for coupling to a “Clack” reporter moiety or protecting group), such as the iodoacetamide PEG azide compound shown in Row A of TABLE 2. A cysteine-selective “Click” label may comprise an iodoacetamide as a first reactive group and a norbornene as a second reactive group, such as the reactant shown in Row B of TABLE 2. Such a reagent may be synthesized by coupling a norbornene amine with an iodoacetamide N-hydroxysuccinamide ester. A cysteine-selective “Click” label may comprise an iodoacetamide as a first reactive group and an aldehyde as a second reactive group, such as 2-iodo-N-(3-oxopropyl) acetamide (as shown in Row C of TABLE 2). Such a compound may be generated by coupling an N-hydroxysuccinamide ester with an amine comprising a geminal diether configured to hydrolyze to an aldehyde. A cysteine-selective label may comprise a first reactive group for coupling to cysteine but lack a second reactive group (e.g., the label may be a “dummy” label), and therefore be unable to couple to a “Clack” reporter moiety or protecting group) reagent. An example of such a reagent may be iodoacetamide, as shown in TABLE 2 Row D.

A lysine-selective “Click” label may comprise an N-hydroxysuccinamide ester as a first reactive group and a norbornene as a second reactive group, such as the reagent shown in Row F of TABLE 2. A lysine-selective “Click” label may comprise an N-hydroxysuccinamide ester as a first reactive group and a geminal diether as a second reactive group, such as the reagent shown in Row G of TABLE 2. Such a reagent may be generated by coupling 1-hydroxypyrrolidine-2,5-dione to the carboxylic acid of a compound comprising a geminal diether. A lysine-selective label may comprise a first reactive group for coupling to lysine but lack a second reactive group for coupling to a “Clack” reporter moiety or protecting group. An example of such a reagent may be an activated ester, such as the compound shown in Row H of TABLE 2.

A carboxylate-selective (e.g., selective for aspartate and glutamate side chain carboxylates) “Click” label may comprise an amine as a first reactive group and an azide as a second reactive group, such as the reagent shown in Row I of TABLE 2. A carboxylate-selective “Click” label may comprise an amine as a first reactive group a norbornene as a second reactive group, such as the reagent shown in Row J of TABLE 2. A carboxylate-selective “Click” label may comprise an amine as a first reactive group a geminal diether as a second reactive group such as the reagent shown in Rows K and L of TABLE 2. A carboxylate-selective label may comprise a first reactive group for coupling to a carboxylate but lack a second reactive group for coupling to a “Clack” reporter moiety or protecting group. An example of such a reagent may be an alkyl amine, such as the compound shown in Row M of TABLE 2. A phosphoserine-, phosphothreonine-, and/or glycosylation-selective “Click” reagent may comprise a disulfide as a first reactive group and an azide, a norbornene, a geminal diether, or an aldehyde as a second reactive group, as shown in Rows N-R of TABLE 2. A phosphoserine-, phosphothreonine-, and/or glycosylation-selective “Click” reagent may comprise a disulfide as a first reactive group and may lack a second reactive group.

TABLE 2
Examples of “Click” Labels

| “CLICK” | | | |
|---|---|---|---|
| LABEL | | | COMMERCIAL AVAILABILITY & |
| TYPE | ROW | “CLICK” LABEL | REAGENTS FOR “CLICK” LABEL SYNTHESIS |
| CYSTEINE- “CLICK” LABELS | A | 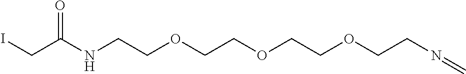 [SMILES: C=NCCOCCOCCOCCNC(=O)CI] | COMMERCIALLY AVAILABLE |
| | B | 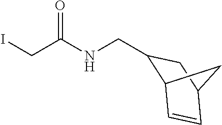 [SMILES: O=C(CI)NCC1CC2C=CC1C2] | 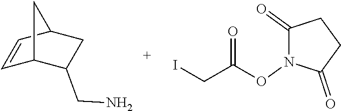 [SMILES: NCC1CC2C=CC1C2 ; O=C(CI)ON1C(=O)CCC1=O] |
| | C | 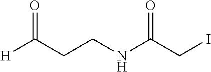 | 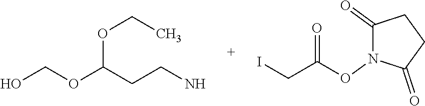 |
| | D | 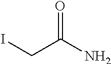 | COMMERCIALLY AVAILABLE |
| LYSINE- “CLICK” LABELS | E | 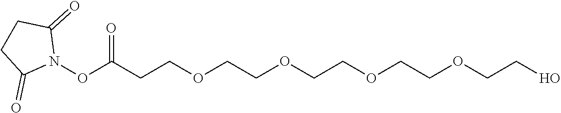 [SMILES: O=C(CCOCCOCCOCCOCCO)ON1C(=O)CCC1=O] | COMMERCIALLY AVAILABLE |
| | F | 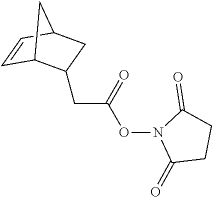 [SMILES: O=C(CC1CC2C=CC1C2)ON1C(=O)CCC1=O] | COMMERCIALLY AVAILABLE |
| | G | 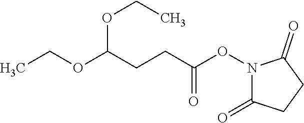 [SMILES: CCOC(CCC(=O)ON1C(=O)CCC1=O)OCC] | 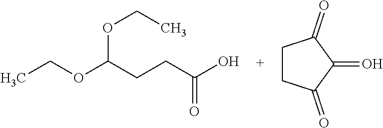 |
| | H | 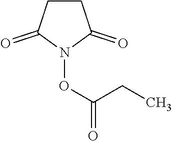 | COMMERCIALLY AVAILABLE |
| CARBOXYLATE- “CLICK” LABELS | I | 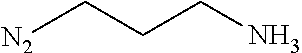 | COMMERCIALLY AVAILABLE |
| | J | 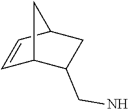 | COMMERCIALLY AVAILABLE |
| | K | 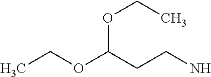 | COMMERCIALLY AVAILABLE |
| | L | | COMMERCIALLY AVAILABLE |
| | M | 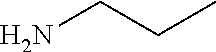 | COMMERCIALLY AVAILABLE |
| PHOSPHOSERINE, PHOSPHOTHREONINE, & | N | 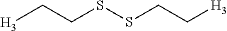 | COMMERCIALLY AVAILABLE |
| GLYCOSYLATION- | | | |
| “CLICK” LABELS | | | |
| | O | 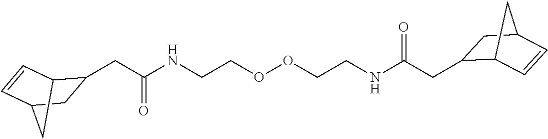 [SMILES: O=C(CC1CC2C=CC1C2)NCCOOCCNC(=O)CC1CC2C=CC1C2] | 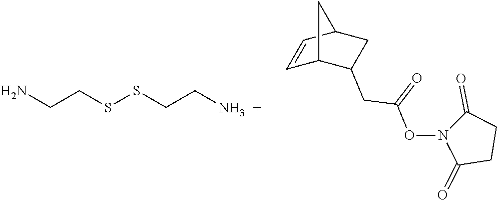 [SMILES: NCCSSCCN ; O=C(CC1CC2C=CC1C2)ON1C(=O)CCC1=O] |
| | P | 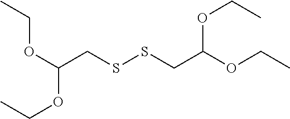 [SMILES: CCOC(CSSCC(OCC)OCC)OCC] | COMMERCIALLY AVAILABLE |
| | Q | 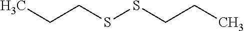 | COMMERCIALLY AVAILABLE |
| | R | | COMMERCIALLY AVAILABLE |

Sample preparation may be improved by labeling a plurality of amino acid residues through series of sequential operations. The present disclosure provides a range of systems to facilitate labeling of multiple amino types. The system may minimize cross-reactivity of amino acids, reporter moieties (e.g., fluorescent molecules (e.g., dyes)), or the decomposition of, for example, sensitive reporter moieties (e.g., fluorescent molecules (e.g., dyes)).

In another aspect, provided herein is a system comprising a peptide and/or protein, wherein said peptide and/or protein: is immobilized to at least one support; and comprises an amino acid coupled to a label, wherein said label comprises (i) a first reactive group that is configured to couple to a second reactive group that is coupled to a reporter moiety configured to emit a signal or (ii) a protecting group configured to prevent coupling between said label and said second reactive group.

In another aspect, provided herein is a system for processing or analyzing a peptide and/or protein (e.g., native and/or non-native), comprising a peptide and/or protein comprising an amino acid coupled to a first reactive group and a support coupled to a second reactive group, wherein the first reactive group is configured to couple to the second reactive group to immobilize the peptide and/or the protein adjacent to the support. In some embodiments, the system is configured to couple a peptide and/or a protein to a support (e.g., a surface). In some embodiments, the system is configured to couple an amino acid residue of a peptide and/or protein to a support (e.g., a surface). The support (e.g., the surface) may comprise a reactive group configured to couple to a functional group coupled to the amino acid residue of the peptide and/or the protein. The peptide and/or the protein may comprise a plurality of amino acids. The peptide and/or the protein may be an oligomer or polymer comprising amino acids or amino acid analogues. The peptide and/or the protein may comprise amino acids that are L-amino acids or D-amino acids. A peptide and/or the protein may be synthetic, recombinant, or naturally occurring. A synthetic peptide and/or synthetic protein may be a peptide and/or a protein that is produced by artificial approaches in vitro. At least one amino acid of the plurality of amino acids may be selected from the group consisting of lysine, cysteine, glutamic acid, aspartic acid, tyrosine, arginine, histidine, threonine, serine, glutamine, asparagine and tryptophan. The plurality of amino acids may comprise one or more amino acids, the one or more amino acid selected from the group consisting of lysine, cysteine, glutamic acid, aspartic acid, tyrosine, arginine, histidine, threonine, serine, glutamine, asparagine and tryptophan. The peptide and/or the protein may comprise one amino acid selected from the group consisting of lysine, cysteine, glutamic acid, aspartic acid, tyrosine, arginine, histidine, threonine, serine, glutamine, asparagine and tryptophan. The plurality of amino acids may comprise a non-natural amino acid. The plurality of amino acids may comprise a D-amino acid.

At least one amino acid of the plurality of amino acids may be coupled to a label. The plurality of amino acids may comprise at least two or more amino acid types. The at least two amino acid or more types may comprise a first amino acid type and a second amino acid type. The first amino acid type may be coupled to a first label. The second amino acid type may be coupled to a second label. The first amino acid type may be coupled to a first label and the second amino acid type may be coupled to a second label. The first label and the second label may each be coupled to a different reporter moiety. The plurality of amino acids may comprise at least one, two, three, four, five, six, seven, eight, nine, ten, eleven, or more amino acid types. The plurality of amino acids may comprise between two and twenty amino acid types. The plurality of amino acids may comprise between 4 and 18 amino acid types. The plurality of amino acids may comprise between 6 and 16 amino acid types. The plurality of amino acids may comprise between 8 and 14 amino acid types. The plurality of amino acids may comprise between 9 and 11 amino acid types. Less than all of the amino acid types of the plurality of amino acids may labelled. Each amino acid type of the at least two amino acid types may be coupled to a different label. The peptide and/or the protein may comprise at least four amino acid types, wherein each amino acid type of said at least four amino acid types are coupled to a different label. Less than all of the plurality of amino acids may be labelled. Each of the plurality of amino acids may be labelled. The plurality of amino acids may comprise at least two amino acid types, and each amino acid type of the at least two amino acid types may be coupled to a different label. The peptide and/or the protein may comprise at least three amino acid types, wherein each amino acid type of said at least three amino acid types are coupled to a different label. The peptide and/or the protein may comprise at least four amino acid types, wherein each amino acid type of said at least four amino acid types are coupled to a different label. The peptide and/or the protein may comprise at least five or six amino acid types, wherein each amino acid type of said at least five or six amino acid types are coupled to a different label. The peptide and/or the protein may comprise at least eight amino acid types, wherein each amino acid type of said at least eight amino acid types are coupled to a different label. The peptide and/or the protein may comprise at least ten amino acid types, wherein each amino acid type of the at least ten amino acid types are coupled to a different label. The peptide and/or protein may comprise of about ten or more amino acid types, wherein each amino acid type of the ten or more amino acid types are coupled to a different label. Each label coupled to a different amino acid type may independently be coupled to a reporter moiety configured to emit a signal corresponding to each amino acid type. In some cases, the majority of the plurality of amino acids are labelled. In some cases, the number of amino acids that are labeled can be from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. In some cases, the number of amino acids that are labeled can be at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more. In some cases, the number of amino acids that are labeled can be at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, or less. In some cases, the number of amino acids that are labeled can be about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some cases, the majority of the plurality of amino acids are unlabeled. In some cases, the number of amino acids that are unlabeled can be from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. In some cases, the number of amino acids that are unlabeled can be at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more. In some cases, the number of amino acids that are unlabeled can be at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 1%, or less. In some cases, the number of amino acids that are unlabeled can be about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The amino acid that can be coupled to a label may be an amino acid selected from the group consisting of lysine, cysteine, glutamic acid, aspartic acid, tyrosine, arginine, histidine, threonine, serine, proline, asparagine, glutamine, and tryptophan. The amino acid that is coupled to a label may comprise a post-translational modification. The post translational modification can be glycosylation, acetylation, alkylation, biotinylation, glutamylation, glycosylation, isoprenylation, phosphorylation, lipolation, phosphopantetheinylation, sulfation, selenation, amidation, ubiquitination, hydroxylation, nitration, nitrosylation, citrullination, cyclization (such as N-terminal glutamate or glutamine cyclization), and SUMOylation.

The peptide and/or the protein may comprise a plurality of amino acids coupled to a plurality of labels. The plurality of amino acids may comprise a plurality of amino acids coupled to a plurality of labels. The plurality of amino acids coupled to a plurality of labels may comprise a first amino acid coupled to a first label and a second amino acid coupled to a second label. The plurality of amino acids may comprise a plurality of first amino acids coupled to a plurality of first labels. The plurality of amino acids may comprise a plurality of second amino acids coupled to a plurality of second labels. The plurality of amino acids may comprise a plurality of third amino acids coupled to a plurality of third labels. The plurality of amino acids may comprise a plurality of fourth amino acids coupled to a plurality of fourth labels. The plurality of amino acids may comprise a plurality of fifth amino acids coupled to a plurality of fifth labels. The plurality of amino acids may comprise a plurality of sixth amino acids coupled to a plurality of sixth labels. The plurality of amino acids may comprise a plurality of seventh amino acids coupled to a plurality of seventh labels. The plurality of amino acids may comprise a plurality of eighth amino acids coupled to a plurality of eighth labels. The plurality of amino acids may comprise a plurality of ninth amino acids coupled to a plurality of ninth labels. The plurality of amino acids may comprise a plurality of ten or more amino acids coupled to a plurality of ten or more labels. The plurality of amino acids may comprise (i) a plurality of first amino acids coupled to a plurality of first labels, (ii) a plurality of second amino acids coupled to a plurality of second labels, (iii) a plurality of third amino acids coupled to a plurality of third labels, (iv) a plurality of fourth amino acids coupled to a plurality of fourth labels, (v) a plurality of fifth amino acids coupled to a plurality of fifth labels, (vi) a plurality of sixth amino acids coupled to a plurality of sixth labels, (vii) a plurality of seventh amino acids coupled to a plurality of seventh labels, (viii) a plurality of eighth amino acids coupled to a plurality of eighth labels, (ix) a plurality of ninth amino acids coupled to a plurality of ninth labels, and/or a (x) a plurality of ten or more amino acids coupled to a plurality of ten or more labels. The first label, or the plurality thereof, may couple to the first amino acid, or the plurality thereof. The second label, or the plurality thereof, may couple to the second amino acid, or the plurality thereof. The third label, or the plurality thereof, may couple to the third amino acid, or the plurality thereof. The fourth label, or the plurality thereof, may couple to the fourth amino acid, or the plurality thereof. The fifth label, or the plurality thereof, may couple to the fifth amino acid, or the plurality thereof. The sixth label, or the plurality thereof, may couple to the sixth amino acid, or the plurality thereof. The seventh label, or the plurality thereof, may couple to the seventh amino acid, or the plurality thereof. The eighth label, or the plurality thereof, may couple to the eighth amino acid, or the plurality thereof. The ninth label, or the plurality thereof, may couple to the ninth amino acid, or the plurality thereof. The ten or more labels, or the plurality thereof, may couple to the ten or more amino acids, or the plurality thereof. At least one label of the plurality of labels may be coupled to a specific amino acid type of the plurality of amino acids. For example, one label of the plurality of labels may be coupled to a lysine, a cysteine, a glutamic acid, an aspartic acid, a tyrosine, an arginine, a histidine, a threonine, a serine, a glutamine, an asparagine, or a tryptophan. A label may comprise a first reactive group that is configured to couple to a second reactive group. The first reactive group may be selected from the group consisting of an azide, an alkyne, an alkene, an aldehyde, a ketone, a tetrazine, a thiol, a dithiol, a cyclooctene, and norbornene. The second reactive group may be selected from the group consisting of an azide, an alkyne, an alkene, an aldehyde, a ketone, a tetrazine, a thiol, a dithiol, a cyclooctene, and norbornene. The first reactive group may be selected from the group consisting of an azide, an alkene, an aldehyde, a ketone, and a tetrazine. The first reactive group may be a strained alkyne. The second reactive group may be selected from the group consisting of an azide, a thiol, a dithiol, a cyclooctene, an alkene, an aldehyde, a ketone, a tetrazine, a norbornene, and an alkyne. The second reactive group may be a strained alkyne.

At least one label of the plurality of labels may be configured to react with a specific second reactive group coupled to a specific reporter moiety. The first reactive group may be selected from the group consisting of an alkyne, a thiol, a dithiol, and a cyclooctene, and the second reactive group may be selected from the group consisting of an alkyne, an azide, a thiol, a dithiol, a cyclooctene, an alkene, an aldehyde, a ketone, a tetrazine, and a norbornene. The first reactive group may be configured to react to a particular second reactive group. For example, the first reactive group may be an azide and the second reactive group may be an alkyne, the first reactive group may be an alkyne and the second reactive group may be an azide, the first reactive group may be an alkene and the second reactive group may be a thiol, the first reactive group may comprise an carbonyl (e.g., a ketone or an aldehyde) and the second reactive group may be an dithiol, the first reactive group may be a tetrazine and the second reactive group may be a cyclooctene (e.g., trans-cyclooctene).

The at least one label that couples to the amino acid, or plurality thereof, may be coupled to a reporter moiety. The reporter moiety may be configured to emit a signal upon excitation. The signal can be a detectable signal. For example, the signal can be an optical signal, such as a fluorescent or phosphorescent signal. The optical signal may be produced by a dye. The reporter moieties may also produce non-optically detectable signals. For example, a reporter moiety may produce an electrical signal, a radioactive signal or a chemical signal. The reporter moiety may be coupled to a spacer. The spacer may adjoin a reporter moiety and a second reactive group. A reporter may be configured to react with the label. The reporter may comprise a reporter moiety and a reactive group (e.g., a second reactive group). The reporter may comprise a reporter moiety, a reactive group (e.g., a second reactive group), and a spacer.

Alternatively, in some cases, the label can be detected by coupling to an additional reporter moiety. In some cases, the additional reporter moiety can be an antibody. In some cases, the additional reporter moiety can be a nucleic acid (e.g., DNA and/or RNA) molecule. In some cases, the nucleic acid molecule can be a barcode.

The at least one support may comprise a bead, a polymer matrix, an array, or any combination thereof. The at least one support may comprise a bead, a polymer matrix, or an array. The at least one support may comprise a bead and an array. The at least one support can be a bead. The at least one support can be an array. The array can be a surface. The array can be a slide. The slide can be a microscopic slide. The at least one support can be a microscopic slide. The at least one support can be a polymer matrix.

The support may comprise a solid support or a semi-solid support. The solid support or semi-solid support may be a bead. The bead may be a gel bead. The bead may be a polymer bead. The support may be a resin. Non-limiting supports may comprise, for example, agarose, sepharose, polystyrene, polyethylene glycol (PEG), or any combination thereof. The support may be a polystyrene bead. The support may include functional groups, such as, for example, amines, sulfhydryls, acids, alcohols, bromides, maleamides, succinimidyl esters (NHS), sulfosuccinimidyl esters, disulfides, azides, alkynes, isothiocyanates (ITC), or combinations thereof. The support may be a PEGA resin. The support may be an amino PEGA resin. The support may comprise an amine group. The support may include protected functional groups, such as, for example, Boc, Fmoc, alkyl ester, Cbz, or combinations thereof. The bead may contain a metal core. The bead may be a polymer magnetic bead. The polymer magnetic bead may comprise a metal-oxide. The support may comprise at least one iron oxide core.

An N-terminus, a C-terminus, a non-terminal amino acid, or any combination thereof, of the peptide and/or the protein can be coupled to the at least one support. The N-terminus and the C-terminus of the peptide and/or the protein can be coupled to the at least one support. The N-terminus of the peptide may be coupled to one support and the C-terminus of the peptide and/or the protein can be coupled to another support. The N-terminus of the peptide and/or the protein may be coupled to a bead. The C-terminus of the peptide and/or the protein may be coupled to a slide. The N-terminus of the peptide and/or the protein may be coupled to a bead and the C-terminus may not be coupled to a support. The C-terminus of the peptide and/or the protein may be coupled to a slide and the N-terminus of the peptide and/or the protein may not be coupled to a support. The N-terminus of the peptide and/or the protein may be coupled to a bead and the C-terminus of the peptide and/or the protein may be coupled to a slide.

The N-terminus of the peptide and/or the protein can be coupled to the at least one support. The N-terminus of the peptide and/or the protein can be coupled to a cleavable unit. The cleavable unit can be coupled to the at least one support. The cleavable unit may comprise at least one of (i) a cleavable moiety, (ii) an aldehyde, (iii) said at least one support, or (iv) a spacer. The cleavable unit may comprise a cleavable moiety. The cleavable moiety may comprise a rink group. The cleavable unit may comprise an aldehyde. The aldehyde may be a pyridinecarboxaldehyde (PCA), or any derivative thereof. The cleavable unit may comprise a spacer. The cleavable unit may comprise at least two of (i) a cleavable moiety, (ii) an aldehyde, (iii) said at least one support, or (iv) a spacer. The cleavable unit may comprise a cleavable moiety and an aldehyde. The cleavable unit may comprise the at least one support and an aldehyde. The cleavable unit may comprise at least three of (i) a cleavable moiety, (ii) an aldehyde, (iii) said at least one support, or (iv) a spacer. The cleavable unit may comprise an aldehyde, the at least one support, and a spacer. The cleavable unit may comprise an aldehyde, the at least one support, and a cleavable moiety. The cleavable unit may comprise a spacer, the at least one support, and a cleavable moiety. The cleavable unit may comprise (i) a cleavable moiety, (ii) an aldehyde, (iii) said at least one support, and (iv) a spacer. The cleavable can be as described in WO2020072907A1. The aldehyde, the spacer, the cleavable moiety, or any combination thereof can be as described in WO2020072907A1.

The C-terminus of the peptide and/or the protein can be modified with an agent configured to couple the C-terminus to at least one support. The agent may comprise an alkyne or an azide, either of which may be configured to couple to at least one support. The C-terminus may comprise an acidic amino acid. The C-terminus may comprise a first acidic residue and a second acidic residue. The first acidic residue may be a C-terminal carboxylic acid. The second acidic residue may be an aspartic acid side chain or a glutamic acid side chain. The first acidic residue and second acidic residue of the C-terminus may be modified. In cases where the C-terminus of the peptide and/or the protein contains two acidic residues, both the first and second acidic residues may be modified by an agent comprising an alkyne or an azide, either of which may be configured to couple to at least one support.

A reporter (or a reporter moiety) for use in the system may, by way of a non-limiting example, emit a detectable or an optical signal (e.g., from a fluorescent dye). However, any number of reporters (or a reporter moieties) as described herein may be used for their various features. As an additional example, a reporter (or a reporter moiety) may emit a radiometric signal, which may be detected by an ionization chamber, a gaseous ionization detector, a Geiger counter, a photodetector, a scintillation counter, or a semiconductor detector, among others. Conversely, a reporter (or a reporter moiety) may not emit a signal at all. Reporters (and reporter moieties) may selectively label specific amino acids by reacting with their side chains, or may detect a post-translational modification to an amino acid. In some examples, a plurality of amino acids may be contained within the peptide and/or the protein, of which many or all may be coupled to a label and/or a reporter (or a reporter moiety).

A peptide and/or a protein, composed of two or more amino acids, may have an N-terminus and a C-terminus. These termini may be separated by one or more amino acids. The N-terminus is a terminal amino acid and may contain a terminal amine. The terminal amine may be unsubstituted, or may be substituted. In some instances, the amine may be cleaved, blocked, functionalized, or otherwise modified. Naturally-occurring peptides and/or proteins generally contain an unsubstituted amine at the N-terminal position. Any amino acid can become an N-terminus following a bond cleaving event. Similarly, the C-terminus is a terminal amino acid and may contain a terminal carboxylic acid. The terminal carboxylic acid may be unsubstituted or substituted. In some instances, the carboxylic acid may be cleaved, blocked, functionalized, or otherwise modified. Naturally-occurring peptides and/or proteins generally contain an unsubstituted carboxylic acid at the C-terminal position. Any amino acid can become a C-terminus following a bond cleavage event. In some examples, as provided herein, the C-terminus may be any amino acid. In other examples, the C-terminus is an acidic amino acid (e.g., glutamate or aspartate). The present disclosure provides for specific cleavage of a first peptide and/or protein at a known site in order to yield a second peptide and/or protein with a specific C-terminal amino acid residue. The C-terminal amino acid, following cleavage, may be an acidic residue. The C-terminal amino acid, following cleavage, may be a non-acidic residue. Similarly, a first peptide and/or protein can be intentionally cleaved to yield a second peptide and/or with a specific N-terminal amino acid residue.

### Fluorosequencing

Various aspects of the present disclosure provide compositions and methods for peptide and/or protein fluorosequencing. A fluorosequencing method disclosed herein can provide peptide and/or protein sequence information at the single molecule level. Fluorosequencing methods are provided in U.S. Pat. No. 9,625,469, U.S. patent application Ser. No. 16/709,903, and U.S. patent application Ser. No. 15/510,962). A method of the present disclosure may subject a peptide and/or protein to fluorosequencing and an additional form of analysis. For example, a molecule of hemoglobin may be interrogated for glycation with immunostaining, and then subsequently digested and subjected to fluorosequencing for sequencing analysis. A method of the present disclosure may subject a peptide and/or a protein to fluorosequencing and an additional form of analysis.

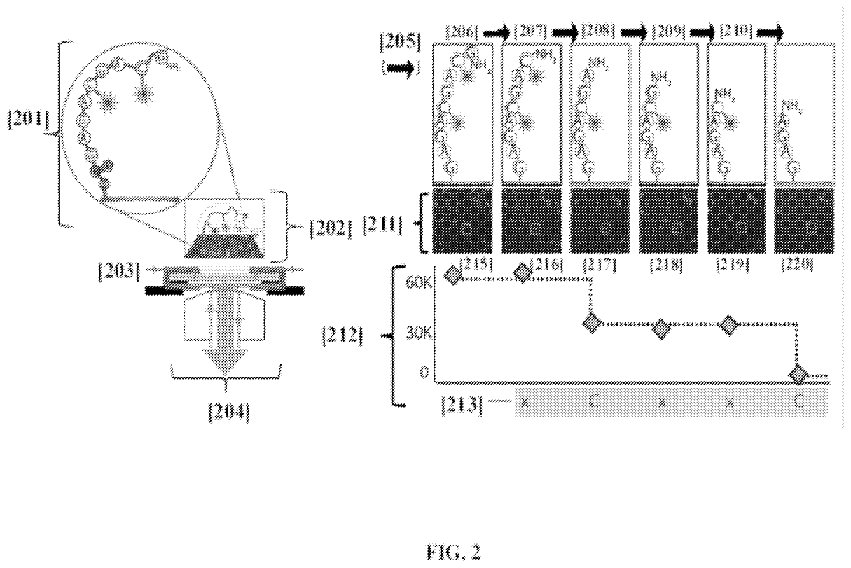

Fluorosequencing can be a parallelizable method for generating positional information of amino acid residues on one or more peptide molecules and/or one or more protein molecules and/or inferring protein identity by matching the positional information of the amino acid residues to a transcript generated reference protein database. In some cases, the transcript used as a reference transcript can be generated by fluorosequencing. Fluorosequencing can generate partial amino acid sequences across millions of peptides and/or proteins by fluorescent labeling of amino acid side chains (e.g., R groups), immobilizing the one or more peptides and/or one or more proteins on a support (e.g., imaging slide), and/or measuring the step decrease in fluorescent intensity on the one or more peptides and/or one or more proteins through cycles of degradation (e.g., Edman degradation). For example, as depicted in FIG. 2, a protein and/or peptide can be immobilized on a support 201. In some cases, one or more peptides and/or one or more proteins can be coupled to the support simultaneously 202. The peptide and/or protein can be coupled to one or more labels. The one or more labels can an amino acid specific-type label. For example, the one or more labels may couple to cysteine amino acid residues. The peptide and/or protein coupled to the one or more labels can be subjected to peptide and/or protein degradation solvents 203. The peptide and/or protein can undergo multiple rounds of degradation (e.g., Edman degradation). In some cases, the peptide and/or protein can undergo from about 2 to about 5 rounds, from about 5 to about 10 rounds, from about 10 rounds to about 15 rounds, from about 15 rounds to about 20 rounds, from about 20 rounds to about 25 rounds, from about 25 rounds to about 30 rounds, from about 30 rounds to about 35 rounds, from about 35 rounds to about 40 rounds, from about 40 rounds to about 45 rounds, from about 45 rounds to about 50 rounds, from about 50 rounds to about 55 rounds, from about 55 rounds to about 60 rounds, from about 60 rounds to about 65 rounds, from about 65 rounds to about 70 rounds, from about 70 rounds to about 75 rounds, from about 75 rounds to about 80 rounds, from about 80 rounds to about 85 rounds, from about 85 rounds to about 90 rounds, from about 90 rounds to about 95 rounds, or from about 95 rounds to about 100 rounds of degradation. In some cases, the peptide and/or protein can undergo at least about 2 rounds, at least about 5 rounds, at least about 10 rounds, at least about 15 rounds, at least about 20 rounds, at least about 25 rounds, at least about 30 rounds, at least about 35 rounds, at least about 40 rounds, at least about 45 rounds, at least about 50 rounds, at least about 55 rounds, at least about 60 rounds, at least about 65 rounds, at least about 70 rounds, at least about 75 rounds, at least about 80 rounds, at least about 85 rounds, at least about 90 rounds, at least about 95 rounds, at least about 100 rounds, or more rounds of degradation. In some cases, the peptide and/or protein can undergo at most about 100 rounds, at most about 95 rounds, at most about 90 rounds, at most about 85 rounds, at most about 80 rounds, at most about 75 rounds, at most about 70 rounds, at most about 65 rounds, at most about 60 rounds, at most about 55 rounds, at most about 50 rounds, at most about 45 rounds, at most about 40 rounds, at most about 35 rounds, at most about 30 rounds, at most about 25 rounds, at most about 20 rounds, at most about 15 rounds, at most about 10 rounds, at most about 5 rounds, at most about 2 rounds, or less rounds of Edman degradation. In some cases, the peptide and/or protein can undergo at about 2 rounds, about 5 rounds, about 10 rounds, about 15 rounds, about 20 rounds, about 25 rounds, about 30 rounds, about 35 rounds, about 40 rounds, about 45 rounds, about 50 rounds, about 55 rounds, about 60 rounds, about 65 rounds, about 70 rounds, about 75 rounds, about 80 rounds, about 85 rounds, about 90 rounds, about 95 rounds, or about 100 rounds of degradation. In each round of degradation 205, the N-terminal amino acids of the peptide and/or protein can be removed. For example, as shown in FIG. 2, in the first round of degradation, a unlabeled glycine is removed 206; in the second round of degradation, a labeled cysteine is removed 207; in the third round of degradation, an unlabeled alanine is removed 208; in the fourth round of degradation, an unlabeled glycine is removed 209; and in the fifth round of degradation, a labeled cysteine is removed 210.

While undergo rounds of degradation, the peptide and/or protein can be detected at a single molecule detection. In some cases, the peptide and/or protein can be detected by a microscopy (e.g., TIRF microscopy) 204. In some cases, each round of degradation can be detected. In some cases, each round of degradation can be detected by fluorescent microscopy 211. The level (e.g., intensity) of each of the one or more labels can be measured at each round of degradation 212. For example, as depicted in FIG. 2, in 212, the y-axis depicts the fluorescence intensity of the labels, while 213 depicts the predicted peptide and/or protein sequence based on the changes in fluorescence intensity after each round of degradation, with “x” representing in unlabeled amino acid and “C” representing a labeled amino acid (e.g., cysteine) When a labeled amino acid residue is removed, the level of the one or more labels can decrease 215-220. The detection of the Edman degradation rounds can generate a pattern of fluorescence. The pattern of fluorescence can be used to generate a predicted amino acid sequence for the protein and/or peptide [213]. Additional operations for sample processing can include N-terminal derivatization, C-terminal derivatization, and/or labeling of peptides and/or proteins on supports. Additionally, sample processing for the fluorosequencing can be high-throughput and/or automated.

A characteristic feature of many fluorosequencing methods is coupling amino acid labels to a peptide and/or protein to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels in the peptides and/or proteins. A fluorosequencing method may comprise labeling from about one to about two different types of amino acid residues, from about two to about three different types of amino acid residues, from about three to about four different types of amino acid residues, from about four to about five different types of amino acid residues, from about five to about six different types of amino acid residues, from about six to about seven different types of amino acid residues, from about seven to about eight different types of amino acid residues, from about eight to about nine different types of amino acid residues, from about nine to about ten different types of amino acid residues, from about ten to about 11 different types of amino acid residues, from about 11 to about 12 different types of amino acid residues, from about 12 to about 13 different types of amino acid residues, from about 13 to about 14 different types of amino acid residues, from about 14 to about 15 different types of amino acid residues, from about 15 to about 16 amino acid residues, from about 16 to about 17 different types of amino acid residues, from about 17 to about 18 different types of amino acid residues, from about 18 to about 19 different types of amino acid residues, or from about 19 to about 20 different types of amino acid residues. In some cases, the fluorosequencing method may comprises labeling at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, or more different types of amino acid residues. In some cases, the fluorosequencing method may comprise labeling at most about 20, at most about 19, at most about 18, at most about 17, at most about 16, at most about 15, at most about 14, at most about 13, at most about 12, at most about 11, at most about ten, at most about nine, at most about eight, at most about seven, at most about six, at most about five, at most about four, at most about three, at most about two, at most about one, or less different types of amino acid residues. In some cases, the fluorosequencing method may comprise labeling about one, about two, about three, about four, about five, about six, about seven, about eight, about nine, about ten, about 11, about 12, about 13, about 14, about 15, about 16, about 17, about 18, about 19, or about 20 different types of amino acid residues. A plurality of amino acid residues may include, for example, an N-terminal amino acid, cysteine, lysine, glutamic acid, aspartic acid, tryptophan, tyrosine, serine, threonine, arginine, histidine, methionine, or any combination thereof. Each of these amino acid residues may be labeled with a different labeling moiety. Multiple amino acid residues may be labeled with the same labeling moiety such as (i) aspartic acid and/or glutamic acid or (ii) serine and threonine.

A label may comprise a detectable moiety. The detectable moiety may be optically detectable (e.g., fluorescent, phosphorescent, luminescent, or light absorbing). The detectable moiety may be electrochemically detectable (e.g., a redox active moiety with a characteristic oxidation or reduction potential). The detectable moiety may comprise a mass tag (e.g., for identification with mass spectrometry. A detectable moiety may identify or determine a label to which it is attached. A plurality of labels may comprise a plurality of detectable moieties which identify or determine labels of the plurality of labels by their type. For example, a method may comprise a plurality of types of labels configured to couple to different amino acids, each comprising a different detectable moiety that uniquely identifies the label by its type.

A label may lack a detectable moiety. A detectable moiety-free label may be used to block an amino acid or amino acid type during a labeling operation, thereby preventing one or more types of amino acids from reacting with a label. For example, a method may comprise coupling a label to cysteine residues before providing a label with specificity for cysteine and lysine, thereby preventing the label from coupling to cysteine residues present in a system.

A label (e.g., a detectable moiety-free label) may reversibly or irreversibly bind to an amino acid type, and thus may be chemically (e.g., by addition of a cleavage reagent) or physically (e.g., by addition of heat or light) decoupled from a target peptide and/or a target protein. A method may thus comprise blocking a first amino acid type (e.g., coupling a detection moiety-free label to cysteine), labeling a second amino acid type (e.g., threonine), unblocking the first amino acid type (e.g., decoupling a label from cysteine), and labeling the first amino acid type. Examples of reversible labels include can include silanes (e.g., trimethylsilane), acetyl groups, benzoyl groups, unsaturated pyran and furan groups, urea-forming groups, carbamate-forming groups, carbonate-forming groups, thiourea-forming groups, thiocarbamate-forming groups, thiocarbonate-forming groups, and derivatives thereof. Examples of irreversible labels can include alkyl groups, oxo-groups, amide-forming groups (e.g., an acyl chloride configured to convert an amine into an amide), and derivatives thereof.

A label may comprise a reactive group. The reactive group may be configured to couple to a reporter moiety, a protecting group, or any combination thereof. A method may comprise coupling a label to an amino acid of a peptide and/or a protein (e.g., coupling a label to each amino acid of a particular type), and then coupling a reporter moiety or protecting group to the label. A method may comprise coupling a plurality of types of labels comprising reactive groups to a plurality of amino acids of a peptide and/or a protein, and coupling a plurality of reporter moieties, protecting groups, or combinations thereof to the labels based on their types. A method may comprise coupling a plurality of types of labels to a plurality of amino acids of a peptide and/or a protein, wherein the plurality of types of labels may comprise labels with reactive groups, labels with reporter moieties (e.g., a cysteine-reactive label coupled to a dye), labels lacking reactive groups and reporter moieties, or any combination thereof.

A label (e.g., a label comprising a reactive group configured to couple to a reporter moiety or protecting group) may reversibly or irreversibly bind to an amino acid type, and thus may be chemically (e.g., by addition of a cleavage reagent) or physically (e.g., by addition of heat or light) decoupled from a target peptide and/or a target protein. A method may thus comprise blocking a first amino acid, labeling a second amino acid type (e.g., threonine), unblocking the first amino acid type, and labeling the first amino acid type. Examples of reversible labels include can include silanes (e.g., trimethylsilane), acetyl groups, benzoyl groups, unsaturated pyran and furan groups, urea-forming groups, carbamate-forming groups, carbonate-forming groups, thiourea-forming groups, thiocarbamate-forming groups, thiocarbonate-forming groups, and derivatives thereof. Examples of irreversible labels can include alkyl groups, oxo-groups, amide-forming groups (e.g., an acyl chloride configured to convert an amine into an amide), and derivatives thereof.

Labeling specificity can be a major challenge for a fluorosequencing method. In many cases, a label may comprise reactivity toward a plurality of amino acid types. For example, some maleimide labels can react with cysteine, lysine, and N-terminal amines. A number of strategies may be employed to utilize or prevent such cross-reactivity. A method may comprise sequential amino acid labeling, for example to ensure that a multi-specific label is added to a system after one or more amino acid types with which the multi-specific label is configured to couple are chemically blocked or labeled, and therefore unable to react with the multi-specific label.

Discriminating between comparably reactive amino acid residues can require precise ordering of labeling operations. In the above maleimide example, lysine may be discriminated from cysteine by first reacting cysteine with a cysteine specific labeling operation (e.g., blocking cysteine in an iodoacetamide coupling operation performed at pH 7-8), thereby preventing further cysteine labeling in a subsequent lysine labeling operation. A method may comprise cysteine labeling prior to lysine labeling. A method may comprise cysteine labeling prior to glutamate labeling. A method may comprise cysteine labeling prior to aspartate labeling. A method may comprise cysteine labeling prior to tryptophan labeling. A method may comprise cysteine labeling prior to tyrosine labeling. A method may comprise cysteine labeling prior to serine labeling. A method may comprise cysteine labeling prior to threonine labeling. A method may comprise cysteine labeling prior to histidine labeling. A method may comprise cysteine labeling prior to arginine labeling. A method may comprise lysine labeling prior to glutamate labeling. A method may comprise lysine labeling prior to aspartate labeling. A method may comprise lysine labeling prior to tryptophan labeling. A method may comprise lysine labeling prior to tyrosine labeling. A method may comprise lysine labeling prior to serine labeling. A method may comprise lysine labeling prior to threonine labeling. A method may comprise lysine labeling prior to arginine labeling. A method may comprise carboxylate side chain (e.g., glutamate and aspartate side chain) labeling prior to tryptophan labeling. A method may comprise carboxylate side chain (e.g., glutamate and aspartate side chain) labeling prior to tyrosine labeling. A method may comprise carboxylate side chain (e.g., glutamate and aspartate side chain) labeling prior to serine labeling. A method may comprise carboxylate side chain (e.g., glutamate and aspartate side chain) labeling prior to threonine labeling. A method may comprise carboxylate side chain (e.g., glutamate and aspartate side chain) labeling prior to histidine labeling. A method may comprise carboxylate side chain (e.g., glutamate and aspartate side chain) labeling prior to arginine labeling. A method may comprise at least 2, at least 3, at least 4, at least 5, or at least 6 amino acid labeling operations performed in a sequence configured to minimize or prevent label cross-reactivity (e.g., labeling more than the intended type or types of amino acids).

Fluorosequencing may comprise removing peptides and/or proteins through techniques such as Edman degradation following or preceding subject peptide and/or protein detection. Sequential peptide and/or protein removal may generate sequence or position-specific information. For example, a reduction in fluorescence following an N-terminal amino acid removal operation may indicate that a labeled amino acid, and thus that a specific type of amino acid, was disposed at a peptide and/or protein N-terminal. Removal of each amino acid residue can be carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide and/or the protein chain. In situations where Edman degradation is used, the amino acid residue at the N-terminus of the peptide chain and/or the protein chain is removed.

A labeling moiety used in the instant application may be configured to withstand conditions for removing one or more of the amino acid residues. Some non-limiting examples of potential labeling moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5)6-napthofluorescein. The labeling moiety may be a fluorescent peptide or protein or a quantum dot.

Fluorosequencing may comprise removing peptides and/or proteins through techniques such as chemical cleavage, Edman degradation, or other forms of enzymatic cleavage following or preceding subject peptide and/or protein detection. Sequential peptide and/or protein removal may generate sequence or position-specific information. For example, a reduction in fluorescence following an N-terminal amino acid removal operation may indicate that a labeled amino acid, and thus that a specific type of amino acid, was disposed at a peptide and/or a protein N-terminal. Removal of each amino acid residue can be carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide and/or the protein chain. In situations where Edman degradation is used, the amino acid residue at the N-terminus of the peptide and/or the protein chain is removed.

A label, reporter moiety, or protecting group of the present disclosure may be configured to withstand conditions for removing one or more of amino acid residues from a peptide and/or a protein. Some non-limiting examples of potential reporter moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5) 6-napthofluorescein. A reporter moiety may comprise fluorescent peptide and/or protein (e.g., green fluorescent protein or a variant thereof) or an optically detectable material, such as a carbon nanotube, a nanorod, or a quantum dot. Peptide and/or protein detection or imaging may comprise immobilizing the peptide and/or the protein on a surface. The peptide and/or protein may be immobilized to the surface by coupling a peptide-derived and/or protein-derived cysteine residue, the peptide and/or the protein N-terminus, or the peptide and/or the protein C-terminus with the surface or with a reagent coupled to the surface. The peptide and/or protein may be immobilized by reacting the cysteine residue with the surface or with a capture reagent coupled to the surface.

A sequencing technique may comprise imaging the peptide and/or protein to determine the presence of one or more labels or reporter moieties (e.g., amino acid labels) coupled to the peptide and/or the protein. The sequencing technique may comprise imaging a plurality of peptides and/or proteins to determine the presence of one or more labels or reporter moieties on individual peptides and/or proteins from among the plurality of peptides and/or proteins. The sequencing technique may comprise imaging at least 103, at least 104, at least 105, at least 106, at least 107, at least 108 or more proteins and/or peptides (e.g., imaging a portion of a surface comprising at least 103 to at least 108 proteins and/or peptides). These images may be taken after each removal of an amino acid residue and thus may enable determination of the location of the specific amino acid in the peptide and/or protein sequence. For example, a C-terminal immobilized peptide and/or protein may comprise a sequence (from N-terminal to C-terminal) of KDDYAGGGAAGKDA (SEQ ID NO: 2, wherein ‘1(’ denotes lysine, ‘D’ denotes aspartate, ‘Y’ denotes tyrosine, ‘A’ denotes alanine, and ‘G’ denotes glycine), and may comprise labels coupled to each lysine and tyrosine residue. A first image comprising the C-terminal immobilized peptide and/or protein may indicate the presence of two lysines and one tyrosine in the peptide and/or protein. The N-terminal amino acid may be removed (e.g., by Edman degradation), such that a second image comprising the C-terminal immobilized peptide and/or protein may indicate the presence of one lysine and one tyrosine in the peptide and/or protein. This process may be repeated until a sequence of KXXXXXXXXXXKX (SEQ ID NO: 3) is identified for the peptide and/or protein, wherein ‘X’ indicates a non-lysine, non-tyrosine amino acid, ‘K’ indicates a lysine, and ‘Y’ indicates a tyrosine.

A method of the present disclosure can identify or determine the position of a specific amino acid in a peptide and/or a protein sequence. A method may be used to determine the locations of specific amino acid residues in the peptide and/or protein sequence or these results may be used to determine the entire list of amino acid residues in the peptide and/or protein sequence. A method may involve determining the location of one or more amino acid residues in the peptide and/or protein sequence and comparing these locations to known peptide and/or protein sequences, which may identify or determine the entire list of amino acid residues in the peptide and/or protein sequence. For example, identifying or determining the positions of the lysines and cysteines in a 40 amino acid fragment of a human protein and/or peptide may uniquely identify or determine the protein and/or peptide (e.g., one human protein contains the specific pattern of lysine and cysteine residues identified in the 40 amino acid fragment). A method of the present disclosure can indicate the presence of a native protein, non-native protein, non-native polypeptide, and/or non-native peptide in a sample. In some cases, the presence of the native protein, non-native protein, non-native polypeptide, and/or non-native peptide in the sample can indicate the presence of a diseases. The native protein, non-native protein, non-native polypeptide, and/or non-native peptide can be a biomarker for a disease. In some cases, the presence of the native protein, non-native protein, non-native polypeptide, and/or non-native peptide in the sample can diagnose a subject with a disease. A method of the present disclosure can quantify the number of native proteins, non-native protein, non-native polypeptide, and/or non-native peptide in a sample. The number of native proteins, non-native protein, non-native polypeptide, and/or non-native peptide in a sample can correlate with disease severity and/or disease progression. A method of the present disclosure can determine the structure of a native protein, non-native protein, non-native polypeptide, and/or non-native peptide. In some cases, the structure of the native protein, non-native protein, non-native polypeptide, and/or non-native peptide may be unknown and/or partial unknown. In some cases, the method can be used to determine if the native protein, non-native protein, non-native polypeptide, and/or non-native peptide is misfolded.

An imaging method may involve a variety of different spectrophotometric and microscopy methods, such as fluorimetry, diffuse reflectance, interferometric scattering, Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide and/or protein. In some cases, fluorescent microscopy can be the imaging method. In some cases, the fluorescent microscope may be coupled to a CMOS camera. In some cases, the imaging method may be a nanopore sequencing method. Such imaging methods may be used to determine the presence or absence of a label on a specific peptide and/or protein sequence. After repeated cycles of removing an amino acid residue and imaging a subject peptide and/or protein, the position of the labeled amino acid residue can be determined in the peptide and/or the protein.

### Support

Peptide and/or protein detection or imaging may comprise immobilizing the peptide and/or the protein on a surface. The peptide and/or the protein may be immobilized to the surface by coupling a peptide-derived and/or a protein-derived cysteine residue, the peptide and/or the protein N terminus, or the peptide and/or the protein C terminus with the surface or with a reagent coupled to the surface. The peptide and/or the protein may be immobilized by reacting the cysteine residue with the surface or with a capture reagent coupled to the surface. The peptide and/or the protein may be immobilized by coupling the peptide and/or the protein C-terminus or N-terminus with a capture moiety described herein. The peptide and/or the protein may be immobilized on a surface. Detecting the immobilized peptide and/or the immobilized protein may comprise capturing an image comprising the peptide and/or the protein. The image may comprise a spatial address specific to the peptide and/or the protein. A plurality of peptides and/or proteins may be detected in a single image, wherein one or more of the peptides and/or the proteins may comprise a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 μm thick, between 1 and 5 μm thick, between 2 and 10 μm thick, between 5 and 20 μm thick, between 20 and 50 μm thick, between 50 and 200 μm thick, between 200 and 500 μm thick, or greater than 500 μm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that may sequester peptides and/or proteins for sequencing and modified targets for selection. Alternatively, an aminosilane modified surfaces may be used in the methods described herein. The methods may comprise immobilizing the peptides and/or the protein on the surface of beads, resins, gels, quartz particles, glass beads, or combinations thereof. In some non-limiting examples, the methods contemplate using peptides and/or proteins that have been immobilized on the surface of Tentagel® beads, Tentagel® resins, or other similar beads or resins. The surface used herein may be coated with a polymer, such as polyethylene glycol. The surface may be amine functionalized or thiol functionalized.

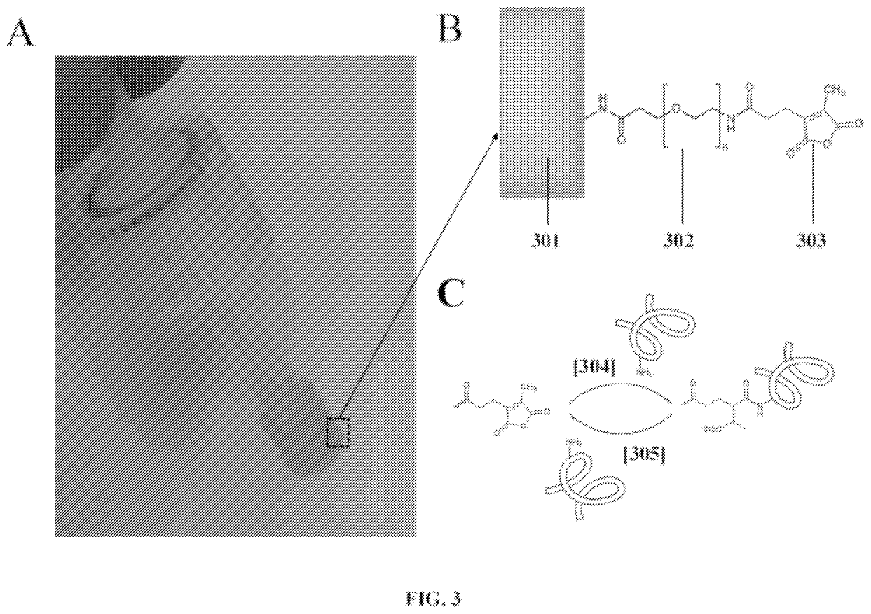

The support can be modified with a capture reagent. In some cases, the capture reagent is a maleic anhydride reagent (e.g., dialkyl anhydride). In some cases, the maleic anhydride reagent may be coupled to a support and/or semi-support. A maleic anhydride reagent may utilize a citraconic anhydride-like reaction with amines for protein-polymer conjugation. For example, a method, system, and/or kit of the present disclosure may provide a bifunctional dialkyl maleic anhydride for coupling to different supports using amide chemistry and/or capture and stabilization of natively folded proteins. As depicted in FIG. 3, for example, the support [301] may be functionalized with a PEG spacer [302] and a maleic anhydride moiety [303]. Such a system may be configured to covalently couple to a surface amine of a protein. The covalent coupling of the capture reagent and the proteins and/or peptides may be reversible. For example, a maleic anhydride reagent may covalently couple to a protein and/or peptide at pH 8 and may uncouple from (e.g., release) the protein and/or peptide at pH<4 [305]. A protein may retain a native folding state through coupling and/or uncoupling with a maleic anhydride reagent. A protein may retain enzymatic activity through coupling and/or uncoupling with a maleic anhydride reagent.

Fluorosequencing may comprise use of a support for the capture of proteins and/or peptides. As the method can comprise sequential labeling of amino acid side chains (e.g., R groups), the proteins and/or peptides can be sequentially contacted with reagents and then the reagents may be removed during the labeling process (e.g., enhance selectivity of the reactions). In some cases, a support may be configured for ease of use by an automated system. In some cases, a support may be configured to allow one or more labeling chemistries. A support may comprise a number of different polymers and architectures (e.g., porous resins and/or magnetic beads). In some cases, a support may be configured to prevent non-specific binding with analytes (e.g., peptides, proteins, nucleic acids, saccharides, lipids, metabolites, and/or isoprenoids) from a biological sample. In some cases, a support may be configured for high capture efficiency. In some cases, the support may be configured for high capture efficiency through modification with a capture reagent. In some cases, the support can have a capture efficiency from about 80% to about 100%. In some cases, the support can have a capture efficiency of at least about 80%, at least about 85%, at least about 90%, at least about 95%, at least about 98%, at least about 99%, or more. In some cases, the support can have a capture efficiency of at most about 99%, at most about 98%, at most about 95%, at most about 90%, at most about 85%, at most about 80%, or less. In some cases, the support can have a capture efficiency of about 80%, about 85%, about 90%, about 95%, about 98%, about 99%, or about 100%. A solid support may comprise a lantern. The lantern may provide advantages for solid-phase chemistry (e.g., increasing ease of sample handling and/or automation). The lantern may be functionalized with a spacer (e.g., PEG). The lantern may comprise a polymer (e.g., polyamide polymer). The lantern may comprise a pin (e.g., polypropylene pin). In some cases, the pin can be the location where the native protein, non-native protein, non-native polypeptide, and/or non-native peptide is coupled to on the support. In some cases, the polymer may be coupled to the pin. The polymer and/or the pin may be functionalized with a spacer. The polymer and/or pin may be functionalized for peptide and/or protein immobilization. The polymer and/or the pin may be configured to not bind (e.g., not absorb) non-target analytes (e.g., lipids, sugars, vitamins, minerals, nucleic acids). For example, the polymer and/or pin may be configured such that background analytes may be removed with a wash. Such a wash may comprise incubating a target species (e.g., a peptide and/or protein) with the lantern (e.g., in a cryovial) and washing the lantern by sequentially moving it into containers (e.g., vials) with different wash solvents (e.g., water and/or methanol). Furthermore, a target species captured on a support can be transported under dry conditions at room temperature and later recovered (e.g., by releasing the target species from the support. In some cases, residual enzymatic activities, such as those of proteases and phosphatases, are eliminated by immobilization of a target species. In some cases, the removal of metabolites (e.g., through a wash operation) improves the accuracy of analysis (e.g., identifying or determining post-translation modifications and protein forms of the target proteins).

In some cases, the support can be used to capture (e.g., bind to the proteins and/or peptides) and release (e.g., detach from the proteins and/or peptides). In some cases, the support can capture and release at least about 70%, at least about 75%, at least about 80%, at least about 85%, at least about 90%, at least about 95%, at least about 98%, at least about 99% of proteins and/or peptides. In some cases, the support can capture and release at most about 99%, at most about 98%, at most about 95%, at most about 90%, at most about 85%, at most about 80%, at most about 75%, at most about 70%, or less of proteins and/or peptides. In some cases, the support can capture and release about 70%, about 75%, about 80%, about 85%, about 90%, about 95%, about 98%, about 99%, or about 100% of proteins and/or peptides.

In some cases, the enzymatic activity of the proteins and/or peptides can be retained through the capture and release. In some cases, at least about 60%, at least about 65%, at least about 70%, at least about 75%, at least about 80%, at least about 85%, at least about 90%, at least about 95%, at least about 98%, at least about 99%, or more of proteins and/or peptides can retain enzymatic activity through the capture and release. In some cases, at most about 99%, at most about 98%, at most about 95%, at most about 90%, at most about 85%, at most about 80%, at most about 75%, at most about 70%, at most about 65%, at most about 60%, or less of proteins and/or peptides can retain enzymatic activity through the capture and release. In some cases, the enzymatic activity of the proteins and/or peptides can be retained through the capture and release. In some cases, about 60%, about 65%, about 70%, about 75%, about 80%, about 85%, about 90%, about 95%, about 98%, about 99%, or about 100% of proteins and/or peptides can retain enzymatic activity through the capture and release. In some cases, the native structure of the proteins can be retained through the capture and release. In some cases, at least about 60%, at least about 65%, at least about 70%, at least about 75%, at least about 80%, at least about 85%, at least about 90%, at least about 95%, at least about 98%, at least about 99%, or more of proteins can retain their native structure through the capture and release. In some cases, at most about 99%, at most about 98%, at most about 95%, at most about 90%, at most about 85%, at most about 80%, at most about 75%, at most about 70%, at most about 65%, at most about 60%, or less of proteins can retain their native structure through the capture and release. In some cases, about 60%, about 65%, about 70%, about 75%, about 80%, about 85%, about 90%, about 95%, about 98%, about 99%, or about 100% of proteins can retain their native structure through the capture and release.

### Sequencing

A sequencing technique described herein may involve imaging the peptide and/or protein to determine the presence of one or more labeling moieties (e.g., amino acid labels) coupled to the peptide and/or the protein. The sequencing technique may comprise imaging a plurality of peptides and/or proteins to determine the presence of one or more labeling moieties on individual peptides and/or proteins from among the plurality of peptides and/or proteins. The sequencing technique may comprise imaging at least 103, at least 104, at least 105, at least 106, at least 107, at least 108 or more proteins and/or peptides (e.g., imaging a portion of a surface comprising at least 103 to at least 108 proteins and/or peptides). These images may be taken after each removal of an amino acid residue and thus may enable determination of the location of the specific amino acid in the peptide and/or protein sequence. For example, a C-terminal immobilized peptide and/or protein may comprise a sequence (from N-terminal to C-terminal) of KDDYAGGGAAGKDA (wherein ‘K’ denotes lysine, ‘D’ denotes aspartate, ‘Y’ denotes tyrosine, ‘A’ denotes alanine, and ‘G’ denotes glycine), and may comprise labels coupled to each lysine and tyrosine residue. A first image comprising the C-terminal immobilized peptide and/or protein may indicate the presence of two lysines and one tyrosine in the peptide and/or the protein. The N-terminal amino acid may be removed (e.g., by Edman degradation), such that a second image comprising the C-terminal immobilized peptide and/or protein may indicate the presence of one lysine and one tyrosine in the peptide and/or the protein. This process may be repeated until a sequence of KXXYXXXXXXXKX is identified for the peptide and/or the protein, wherein ‘X’ indicates a non-lysine, non-tyrosine amino acid, ‘K’ indicates a lysine, and ‘Y’ indicates a tyrosine. A method of the present disclosure can identify or determine the position of a specific amino acid in a peptide and/or protein sequence. A method may be used to determine the locations of specific amino acid residues in the peptide and/or the protein sequence or these results may be used to determine the entire list of amino acid residues in the peptide and/or the protein sequence. A method may involve determining the location of one or more amino acid residues in the peptide and/or the protein sequence and comparing these locations to known peptide and/or protein sequences, which may identify or determine the entire list of amino acid residues in the peptide and/or the protein sequence. For example, identifying or determining the positions of the lysines and cysteines in a 40 amino acid fragment of a human protein and/or peptide may uniquely identify or determine the protein and/or peptide (e.g., one human protein and/or peptide contains the specific pattern of lysine and cysteine residues identified in the 40 amino acid fragment).

An imaging method may involve a variety of different spectrophotometric and microscopy methods, such as fluorimetry, diffuse reflectance, interferometric scattering, Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide and/or a protein. Such imaging methods may be used to determine the presence or absence of a label on a specific peptide and/or protein sequence. After repeated cycles of removing an amino acid residue and imaging a subject peptide and/or protein, the position of the labeled amino acid residue can be determined in the peptide and/or the protein.

### Selective Amino Acid Labeling

Various aspects of the present disclosure provide methods for selectively labeling types (e.g., lysine, tyrosine, or phosphotyrosine) or groups (e.g., carboxylate side chain-containing or aromatic side chain-containing) of amino acids. A composition, system, or method of the present disclosure may selectively label cysteine, lysine, histidine, glutamic acid, aspartic acid, tyrosine, tryptophan, threonine, phosphothreonine, serine, phosphoserine, methionine, arginine, N-terminal amines, C-terminal carboxyl-groups, or any combination thereof. A composition, system, or method may selectively label a group of amino acids, for example, a substituted maleimide reagent may couple to lysine and cysteine residues present in a sample.

The free thiol group of a cysteine side often the most nucleophilic group in a peptide and/or protein (Scheme 1), and thus may promiscuously react with a range of reagents. To prevent such cross-reactivity, thiol side chains are often reacted early in a labeling (e.g., a multi-labeling scheme), thereby blocking them from further reactions. An example of a thiol-selective reaction is an iodoacetamide coupling operation. Such a reaction may be performed in pH ranges which can prevent lysine cross reactivity, such as at a sufficiently low pH to ensure lysine protonation, which may diminish lysine reactivity.

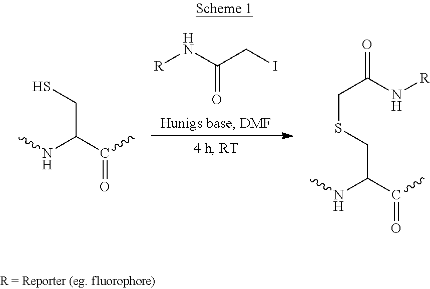

Scheme 2 provides an example of a lysine labeling reaction. The a lysyl amine (e.g., a lysyl butylamine sidechain) can be selectively labeled with an ester (e.g., an NHS ester). This operation may be performed after cysteine labeling in cases where cross-reactivity may be possible.

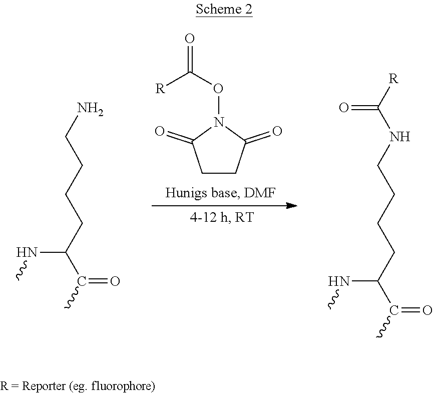

Peptide and/or protein carboxylates may be labeled through amine coupling, an example of which is provided in Scheme 3. Carboxyl-side chains (e.g., those of aspartic acid and glutamic acid) and C-terminal carboxyls can be converted to amides via amine-based nucleophilic substitution. The resulting amides may comprise detectable moieties, chemically inert groups, or reactive handles for further coupling. For example, an amine reagent for carboxylate amidation may comprise an alkyne suitable for a subsequent coupling operation. In some instances, a polypeptide is digested using GluC under pH 8 digestion buffer or a sufficiently similar protease/buffer system such that the cleavage site occurs on the C-terminal-side of an acidic residue (e.g., aspartic acid and glutamic acid). Such a digestion method can generate peptides and/or proteins wherein every carboxyl-residue (e.g., glutamic acid and aspartic acid) is disposed at a peptide and/or a protein C-terminus, thus enabling C-terminal selective amino acid immobilization. Whether the C-terminal carboxylic acid, the side chain carboxylic acid, or both are amidated and immobilized to the support may not affect the function of the systems, methods, and kits as disclosed herein. Alternate reactive groups can be used in place of an alkyne. However, for brevity, the alkyne example is discussed above.

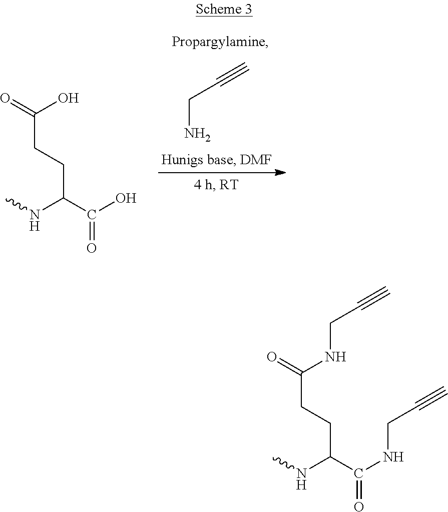

Scheme 4 provides an example of tyrosine-specific labeling. The position adjacent (e.g., ortho to) the tyrosine phenol hydroxyl carbon can be labeled through a two-operation labeling process using a bifunctional diazonium reagent. Following diazo-coupling to tyrosine, a second reagent (such as a dithiolane) may optionally be coupled to the diazo label (e.g., to selectively couple a detectable moiety to the labeled tyrosine). Alternatively, the diazonium reagent may comprise a detectable moiety or may lack chemically reactive handles for further coupling.

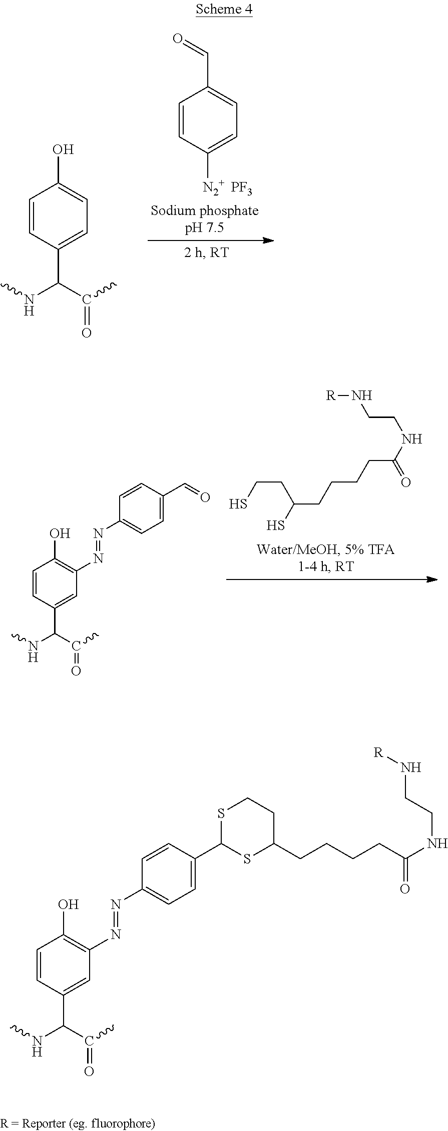

Scheme 5 provides an example of a histidine coupling scheme. A histidine imidazole nitrogen can be labeled through a two-operation labeling process using an alpha-beta unsaturated carbonyl compound, such as 2-cyclohexenone. The alpha-beta unsaturated carbonyl compound may react with histidine in a nucleophilic addition reaction. The alpha-beta unsaturated carbonyl may comprise a detectable moiety. Following histidine coupling, the alpha-beta unsaturated carbonyl may be further coupled to an additional label, such as a dithiolane. Histidine may alternatively be selectively coupled to an epoxide reagent.

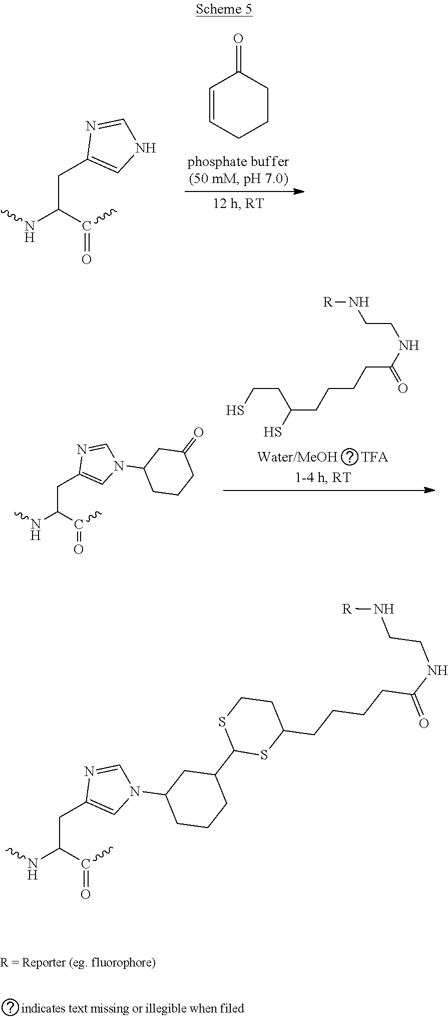

Scheme 6 provides an example of an arginine labeling mechanism. An arginine guanidinium can be acylated (e.g., labeled with an NHS ester with the aid of Barton's base). This example reaction may show cross-reactivity or interference by primary amines (e.g., N-terminus, lysine) or thiols (e.g., cysteine), and may be performed after N-terminal support immobilization and cysteine and lysine labeling in order to prevent or diminish cross-reactivity.

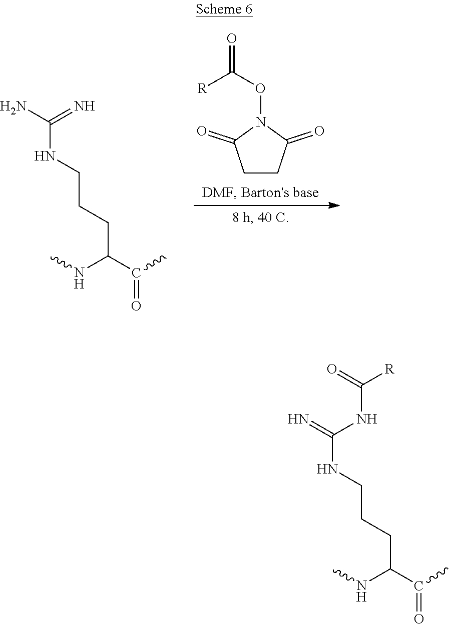

Methionine comprises a relatively low nucleophilicity and can often be selectively labeled by a redox based scheme where an oxaziridine group reacts specifically with a methionine thioether without cross-reacting with cysteine (Scheme 7). The bond formed is stable to reducing agents such as TCEP.

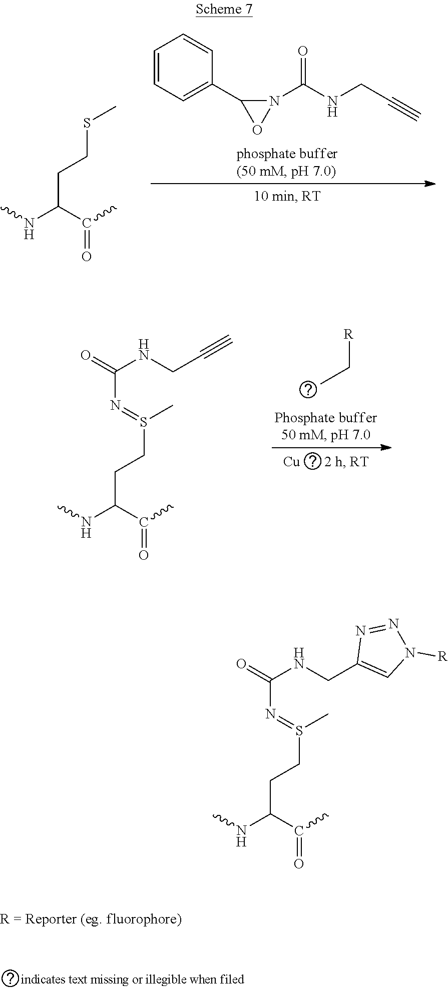

Scheme 8 provides an example of a tryptophan labeling scheme. A tryptophan indole may couple to a diazopropanoate ester, yielding a tertiary amine derivatized tryptophan, The coupling may be metal-catalyst mediated, for example by a dirhodamine (II) tetraacetate complex, which may enhance the selectivity for tryptophan over other amino acid types.

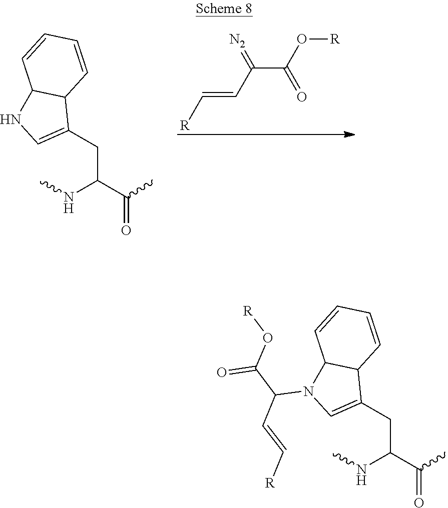

Phosphorylated amino acids such as phosphoserine, phosphotyrosine, or phosphothreonine can be selectively labeled. Such a labeling method may distinguish between types of phosphorylated amino acids. For example, Scheme 9 below provides a phosphoryl beta-elimination followed by a label conjugate addition (e.g., a Michael acceptor reaction) operation for selectively labeling of phosphoserine (pSer) and phosphothreonine (pThr) over other phosphorylated amino acids such as phosphotyrosine (pTyr). A subsequent pan-phospho labeling method can be implemented to label pTyr.

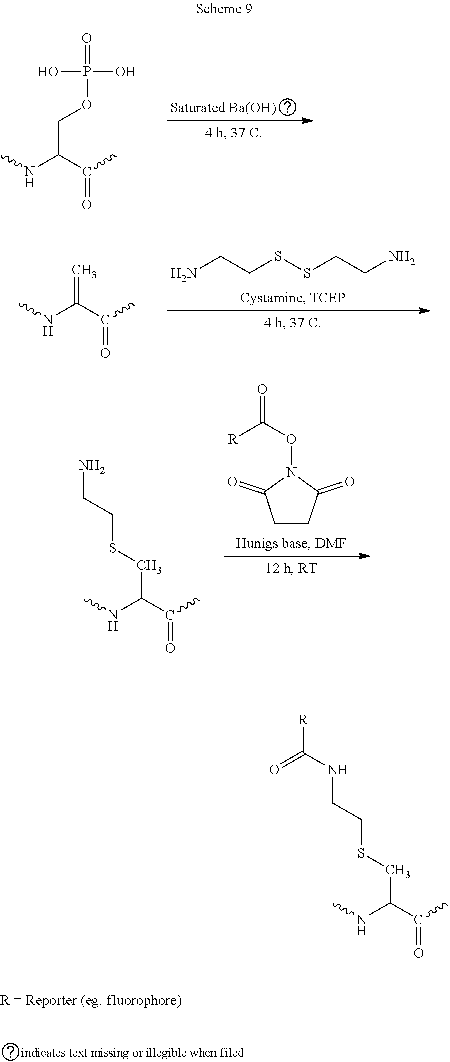

Peptide and/or Protein Degradation

Chemical techniques that allow for the mild and sequential protein degradation conditions can be important for proteomics. Degradation can be used as a method to sequence polymers (e.g., proteins and/or peptides) to determine the order and identity of the amino acids of a polymer. A peptide and/or protein may be subsequently subjected to additional cleavage conditions until the sequence of at least a portion of the peptide and/or protein is identified. The entire sequence of a peptide and/or a protein may be determined using the methods and compositions described herein. Removal of each amino acid residue may be carried out through a variety of techniques including, for example, Edman degradation, organophosphate degradation, or proteolytic cleavage. In some aspects, Edman degradation may be used to remove a terminal amino acid residue. These terminal amino acid residues may be removed from either the C-terminus or the N-terminus of the peptide and/or the protein chain. In some instances, the amino acid residue at the N-terminus of the peptide and/or the protein chain may be removed. A chemical or enzymatic technique for removing a terminal amino acid may remove a defined number of (e.g., exactly one) amino acid. Accordingly, a method for analyzing a peptide and/or a protein may comprise successive degradation and analysis operations, such that the removal of a defined number of amino acids from an N-terminus or C-terminus per operation provides position and sequence specific amino acid identifications during analysis. A chemical or enzymatic technique for removing a terminal amino acid may cleave a peptide and/or a protein at a defined location (e.g., in between two alanine residues).

An Edman degradation method may comprise chemically functionalizing a peptide and/or a protein N-terminus or C-terminus (e.g., to form a thiourea or a guanidinium derivative of an N-terminal amine), and then contacting the functionalized terminal amino acid with a reagent (e.g., a hydrazine), a condition (e.g., a high or low pH or temperature), or an enzyme (e.g., an Edmanase with specificity for the functionalized terminal amino acid) to remove the functionalized terminal amino acid.

A diactivated phosphate or phosphonate may be used for peptide and/or protein cleavage. Such a method may utilize an acid to remove a functionalized amino acid. The diactivated phosphate or phosphonate may be a dihalophosphate ester. In other embodiments, the techniques involve using an enzyme to remove the terminal amino acid residue, such as, for example, an exopeptidase or an Edmanase. For example, a method may comprise derivatizing an N-terminal amino acid of a peptide and/or a protein with a diactivated phosphate, and contacting the peptide and/or the protein with an Edmanase with cleavage activity toward phosphate-functionalized N-terminal amino acids.

Peptide and/or protein cleavage conditions may be achieved with a solvent. The solvent may be an aqueous solvent, organic solvent, or a combination thereof. The solvent may be a mixture of solvents. The solvent may be an organic solvent. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or a polar protic solvent (e.g., isopropanol (IPA), ethanol, methanol, acetic acid, water, etc.). The solvent may be a polar aprotic solvent. The solvent may be DMF. The solvent may be a C₁-C₁₂haloalkane. The C₁-C₁₂haloalkane may be DCM. The solvent may be a mixture of two or more solvents. The mixture of two or more solvents may be a mixture of a polar aprotic solvent and a C₁-C₁₂haloalkane. The mixture of two or more solvents may be a mixture of DMF and DCM. The mixture of solvents may be any combination thereof.

A degradation process may comprise a plurality of operations. For example, a method may comprise an initial operation for derivatizing a terminal amino acid of a peptide and/or a protein, and a subsequent operation for cleaving the derivatized terminal amino acid from the peptide and/or the protein. One such method comprises organophosphorus compound-mediated N-terminal functionalization and removal, and thus provides an alternative to the isothiocyanate (e.g., phenyl isothiocyanate) based processes of some Edman degradation schemes.

An organophosphate-based degradation scheme may comprise dissolving the peptide and/or the protein in an organic solvent or organic solvent mixture (e.g., a mixture of dichloromethane and dimethylformamide) in the presence of an organic base (e.g., triethylamine, N, N-diisopropylethylamine (DIPEA), 1,8-diazabicyclo [5.4.0]undec-7-ene (DBU), pyridine, 1,5-diazabicyclo(4.3.0) non-5-ene, 2,6-di-tert-butylpyridine, imidazole, histidine, sodium carbonate, etc.). The peptide and/or the protein may then be contacted with at least one organophosphorus compound. The cleavage of the peptide and/or protein N-terminus may be initiated through the addition of a weak acid (e.g., formic acid in water). The cleavage of the peptide and/or protein N-terminus may also be initiated with water. The resulting products may include the terminal amino acid of the peptide and/or protein released from the peptide and/or the protein as a phosphoramide and the peptide and/or protein that is shortened by the terminal amino acid residue, which comprises a free N-terminus that can be used to perform a subsequent cleavage reaction.

The reaction mixture may comprise a stoichiometric or an excess concentration of the cleavage compound (e.g., relative to the concentration of peptides and/or proteins to be cleaved). The reaction mixture may comprise at least about 0.001% v/v, about 0.01% v/v, about 0.1% v/v, about 1% v/v, about 5% v/v, about 10% v/v, about 15% v/v, about 20% v/v, about 30% v/v, about 40% v/v, about 50% v/v, or more of the cleavage compound. The reaction mixture may comprise at most about 50% v/v, about 40% v/v, about 30% v/v, about 20% v/v, about 15% v/v, about 10% v/v, about 5% v/v, about 1% v/v, about 0.1% v/v, about 0.01% v/v, about 0.001% v/v, or less of the cleavage compound. The reaction mixture may comprise from about 0.1% v/v to about 20% v/v, about 0.5% v/v to about 10% v/v, or about 1% v/v to about 10% v/v of the cleavage compound. The reaction mixture may comprise about 5% v/v of the cleavage compound.

The reaction may be performed at a temperature of at least about 0° C., about 5° C., about 10° C., about 15° C., about 20° C., about 25° C., about 30° C., about 40° C., about 50° C., about 60° C., about 70° C., or more. The reaction may be performed at a temperature of at most about 70° C., about 60° C., about 50° C., about 40° C., about 30° C., about 25° C., about 20° C., about 15° C., about 10° C., about 5° C., about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature.

The peptide and/or the protein and the cleavage compound may be mixed or incubated for at least about 1 minute, about 5 minutes, about 10 minutes, about 20 minutes, about 30 minutes, about 40 minutes, about 50 minutes, about 60 minutes, about 2 hours, about 3 hours, about 4 hours, about 6 hours, about 8 hours, about 10 hours, about 12 hours, about 16 hours, about 20 hours, about 24 hours, or more. The peptide and/or the protein and the cleavage compound may be mixed or incubated for at most about 24 hours, about 20 hours, about 16 hours, about 12 hours, about 10 hours, about 8 hours, about 6 hours, about 4 hours, about 3 hours, about 2 hours, about 1 hour, about 50 minutes, about 40 minutes, about 30 minutes, about 20 minutes, about 10 minutes, about 5 minutes, about 1 minute, or less. The peptide and/or the protein and the cleavage compound may be mixed or incubated from about 1 minute to about 24 hours, 5 minutes to about 6 hours, 5 minutes to about 2 hours, or 5 minutes to about 30 minutes.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition of synthetic peptides and/or proteins. A sample may comprise a single species or a mixture of species. A biological sample may comprise biomaterial from a single organism, from a colony of genetically near-identical organisms, or from multiple organisms (e.g., enterocytes and microbiota from a human digestive tract). A biological sample may be fractionated (e.g., plasma separated from whole blood), filtered, or depleted (e.g., high abundance proteins such as albumin and ceruloplasmin removed from plasma).

A sample may comprise all or a subset of the biomolecules from the subject, tissue sample, cell culture, cell, or portion thereof. For example, a sample from a subject may comprise the majority of proteins present in that subject, or may comprise a small subset of the proteins from that subject. A biological sample may comprise a bodily fluid such as cerebral spinal fluid, saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which it was sourced) or purified, such that the macromolecule comprises at least 0.5%, at least 1%, at least 2%, at least 3%, at least 4%, at least 5%, at least 7.5%, at least 10%, at least 15%, at least 20%, at least 25%, at least 30%, at least 40%, at least 50%, at least 60%, at least 70%, at least 75%, at least 80%, at least 90%, at least 95%, at least 98%, or at least 99% of a composition by weight (e.g., by dry weight or including solvent). The biological sample may be complex, and may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (polypeptides, peptides, nucleic acids, lipids, small molecules). A biological sample may comprise a plurality of peptides and/or proteins configured for a method of the present disclosure (e.g., digestion, C-terminal labeling, or fluorosequencing).

Methods of the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and buffy coat proteins may be partitioned and separately analyzed) or pooled for common analysis. A biological species may be homogenized, fragmented, or lysed prior to analysis. In some instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides and/or proteins from the resulting lysate, and analyzing the peptides and/or the protein by a fluorosequencing method of the present disclosure. A method may comprise capturing peptides and/or proteins from a sample using a C-terminal capture reagent, and analyzing the peptides and/or the protein (e.g., by a fluorosequencing method).

Methods of the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and the subject of the second analytical method may be derived from the same subject or the same sample. For example, a method may comprise collecting cell free DNA and peptides and/or proteins from a human plasma sample, sequencing the cell free DNA (e.g., to identify or determine a cancer marker), and performing proteomic analysis on the plasma proteins.

### Computer Systems

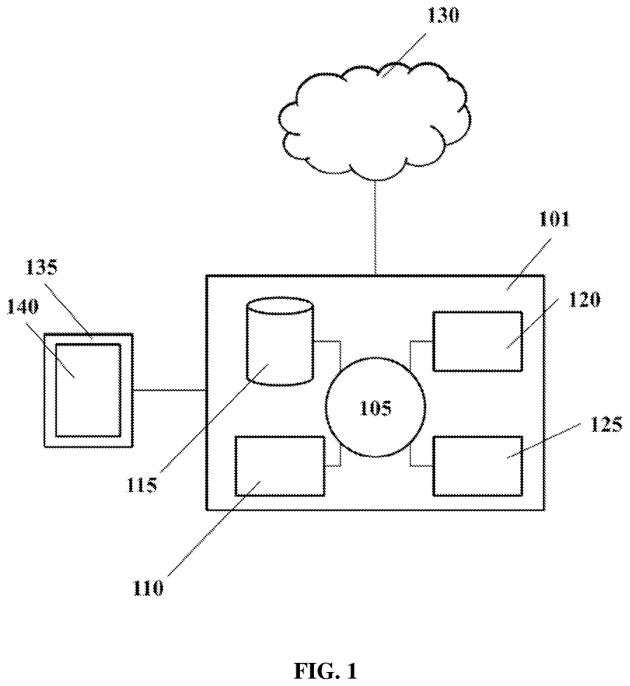

The present disclosure provides computer systems that are programmed to implement methods of the disclosure. FIG. 1 shows a computer system 101 that is programmed or otherwise configured to implement methods or parts of methods disclosed herein, including compiling, analyzing, and displaying data obtained through the present methods. The computer system 101 may regulate various aspects of the present disclosure, such as, for example, detecting and/or quantifying optical signals. The computer system 101 may be an electronic device of a user or a computer system that is remotely located with respect to the electronic device. The electronic device may be a mobile electronic device.

The computer system 101 includes a central processing unit (CPU, also “processor” and “computer processor” herein) 105, which may be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some cases is a telecommunication and/or data network. The network 130 may include one or more computer servers, which may enable distributed computing, such as cloud computing. The network 130, in some cases with the aid of the computer system 101, may implement a peer-to-peer network, which may enable devices coupled to the computer system 101 to behave as a client or a server.

The CPU 105 may execute a sequence of machine-readable instructions, which may be embodied in a program or software. The instructions may be stored in a memory location, such as the memory 110. The instructions may be directed to the CPU 105, which may subsequently program or otherwise configure the CPU 105 to implement methods of the present disclosure. Examples of operations performed by the CPU 105 may include fetch, decode, execute, and writeback.

The CPU 105 may be part of a circuit, such as an integrated circuit. One or more other components of the system 101 may be included in the circuit. In some cases, the circuit is an application specific integrated circuit (ASIC).

The storage unit 115 may store files, such as drivers, libraries and saved programs. The storage unit 115 may store user data, e.g., user preferences and user programs. The computer system 101 in some cases may include one or more additional data storage units that are external to the computer system 101, such as located on a remote server that is in communication with the computer system 101 through an intranet or the Internet.

The computer system 101 may communicate with one or more remote computer systems through the network 130. For instance, the computer system 101 may communicate with a remote computer system of a user (e.g., a fluorosequencing device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine executable or machine readable code may be provided in the form of software. During use, the code may be executed by the processor 105. In some cases, the code may be retrieved from the storage unit 115 and stored on the memory 110 for ready access by the processor 105. In some situations, the electronic storage unit 115 may be precluded, and machine-executable instructions are stored on memory 110.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 may include or be in communication with an electronic display 135 that comprises a user interface (UI) 140 for providing, for example, signal readouts for fluorosequencing. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 105. The algorithm may, for example, determine a correlation using linear and quadratic discriminant analysis (LDA and QDA), Support Vector Machine (SVM), linear discriminant analysis (LDA), quadratic discriminant analysis (QDA), Naive Bayes, Random Forest, or any other suitable method.

### Kits

In another aspect, provided herein is a kit for analyzing a peptide and/or a protein. In some aspects, the kit is for assaying a sequence of a peptide and/or protein. In some aspects, the kit is for determining structure of a protein.

In another aspect, provided herein is a kit for assaying a sequence of a peptide and/or a protein in a sample, comprising a label comprising a first reactive group and/or (i) a second reactive group or (ii) a protecting group, wherein the first reactive group is configured to couple to an amino acid of an amino acid type, wherein the second reactive group is configured to couple to a reporter moiety, comprising a reporter moiety configured to emit a signal, and wherein the protecting group is configured to prevent coupling between the label and the reporter moiety; and instructions for using the label to process the peptide and/or the protein to provide the peptide and/or the protein comprising the amino acid coupled to the label. The first reactive group may be configured to couple to an amino acid of an amino acid type. The second reactive group may be configured to couple to the reporter. The reporter may be coupled to a third reactive group configured to the second reactive group. The reporter may comprise a spacer. The reporter may be configured to emit a signal upon excitation. The reporter may comprise a fluorescent dye. The protecting group may be configured to prevent coupling between the label and the reporter. The protecting group may not emit an optically detectable signal.

The kit may comprise a protein capture agent. The protein capture agent may be configured to couple to an N-terminus, a C-terminus, and/or a non-terminal amino acid residue of the peptide and/or the protein. The protein capture agent may be coupled to a support. The protein capture agent may be a substituted anhydride. The protein capture agent may comprise a support coupled to a cleavable linker. The protein capture agent may be coupled to a support by a cleavable linker. The support may comprise a bead, an array, a slide, a polymer matrix, or any combination thereof. The cleavable linker may be cleavable by an enzyme. The cleavable linker may be a chemically cleavable linker. The cleavable linker may be a photocleavable linker. The cleavable linker may be capable of being cleaved by a change in pH. The cleavable linker may comprise an aldehyde. The aldehyde may be pyridinecarbaldehyde (PCA) or a derivative of PCA.

A capture reagent may react with at least one peptide and/or protein. A capture reagent may react with the N-terminus of at least one peptide and/or protein. A capture reagent may react with the C-terminus of at least one peptide and/or protein. A capture reagent may react with a non-terminal amino acid residue of at least one peptide and/or protein. A capture reagent may react with one peptide and/or protein. A capture reagent may react with the N-terminus of one peptide and/or protein. A capture reagent may react with the C-terminus of one peptide and/or protein. A capture reagent may react with a non-terminal amino acid residue of one peptide and/or protein. Each peptide and/or protein of a cell may be captured by a plurality of capture reagents. The reporter (or reporter moiety) may be configured to emit a signal. The reporter (or reporter moiety) may comprise a dye. The dye may be selected from the group consisting of fluorescent dyes, phosphorescent dyes, chemiluminescent dyes, pigments, and photoswitchable reporters. The reporter (or reporter moiety) may comprise a fluorescent dye. The reporter may be configured to emit the signal upon excitation. The reporter may be a fluorescent protein molecule.

The kit may comprise a surface attachment agent. The surface attachment agent may comprise an alkyne or an azide. The surface attachment agent may be configured to couple to a C-terminus of a peptide and/or a protein. The surface attachment agent may be configured to couple a N-terminus of a peptide and/or a protein. The surface attachment may be configured to couple to a non-terminal amino acid residue of a peptide and/or protein. A kit may comprise a support to which the surface attachment agent attaches. In some cases, the support is a slide. The slide may be a glass slide. The slide may be a microscopic slide. In some cases, the support may be a lantern. The kit may comprise additional agents useful for carrying out a reaction, handling a peptide and/or a protein, or any of the reagents described herein, or performing analysis. A kit may comprise one or more species from the group consisting of proteases, digestion reagents, supports, or any combination thereof. A kit may also comprise small molecules, buffers, and/or solvents useful for carrying out a reaction. A kit may come pre-packaged in a container set. The prepackaging may be a cassette configured to be used in any sequencing platform.

Reagents may be optimized for sample compatibility. A method may comprise selecting a reagent with a low cross-reactivity within a sample. For example, some fluorophores can intercalate into nucleic acids (for example, flavonoid-based dyes can rapidly intercalate into B-DNA present in many samples) and may therefore be excluded from methods which utilize nucleic acid-containing samples. A method may comprise selecting a reagent (e.g., a reporter moiety) which comprises a half-life from about a hour to about 12 hours, from about 12 hours to about 24 hours, from about 24 hours to about 48 hours, from about 48 hours to about 72 hours, or from about 72 hours to about 120 hours at 25° C. in a sample from which an analyte was derived. A method may comprise selecting a reagent which comprises a half-life of at least 1 hour, at least 2 hours, at least 4 hours, at least 6 hours, at least 8 hours, at least 12 hours, at least 16 hours, at least 24 hours, at least 36 hours, at least 48 hours, at least 60 hours, at least 72 hours, at least 90 hours, at least 120 hours, or more at 25° C. in a sample from which an analyte was derived. A method may comprise selecting a reagent which comprises a half-life of at most about 120 hours, at most about 90 hours, at most about 72 hours, at most about 60 hours, at most about 48 hours, at most about 36 hours, at most about 24 hours, at most about 16 hours, at most about 12 hours, at most about 8 hours, at most about 6 hours, at most about 4 hours, at most about 2 hours, at most about 1 hour, or less at 25° C. in a sample from which an analyte was derived. A method may comprise selecting a reagent which comprises a half-life of about 1 hour, about 2 hours, about 4 hours, about 6 hours, about 8 hours, about 12 hours, about 16 hours, about 24 hours, about 36 hours, about 48 hours, about 60 hours, about 72 hours, about 90 hours, or about 120 hours at 25° C. in a sample from which an analyte was derived. A method may comprise selecting a reagent (e.g., a reporter moiety) which comprises a half-life from about a hour to about 12 hours, from about 12 hours to about 24 hours, from about 24 hours to about 48 hours, from about 48 hours to about 72 hours, or from about 72 hours to about 120 hours at 25° C. in a sample in which an analyte is interrogated (e.g., a buffer for fluorosequencing). A method may comprise selecting a reagent which comprises a half-life of at least 1 hour, at least 2 hours, at least 4 hours, at least 6 hours, at least 8 hours, at least 12 hours, at least 16 hours, at least 24 hours, at least 36 hours, at least 48 hours, at least 60 hours, at least 72 hours, at least 90 hours, at least 120 hours, or more at 25° C. in a sample in which an analyte is interrogated (e.g., a buffer for fluorosequencing). A method may comprise selecting a reagent which comprises a half-life of at most 120 hours, at most about 90 hours, at most about 72 hours, at most about 60 hours, at most about 48 hours, at most about 36 hours, at most about 24 hours, at most about 16 hours, at most about 12 hours, at most about 8 hours, at most about 6 hours, at most about 4 hours, at most about 2 hours, at most about 1 hour, or less at 25° C. in a sample in which an analyte is interrogated. A method may comprise selecting a reagent which comprises a half-life of about 1 hour, about 2 hours, about 4 hours, about 6 hours, about 8 hours, about 12 hours, about 16 hours, about 24 hours, about 36 hours, about 48 hours, about 60 hours, about 72 hours, about 90 hours, or about 120 hours at 25° C. in a sample in which an analyte is interrogated. A method may comprise selecting a reagent which comprises tolerance for a pH, a temperature, a salinity, or a reactive species of a sample.

A set of reagents or a kit may also be optimized so that multiple reactions may be performed in a single operation. A kit of the present disclosure may comprise a plurality of “click” labels and/or corresponding “clack” reporter moieties or protecting groups which reactive group pairs amenable for single operation coupling reactions. For example, a kit may be configured for a method in which cysteine residues present in a sample are coupled to a first label, lysine residues present in the sample are coupled to a second label, tryptophan residues present in the sample are coupled to a third label, and then all three labels are coupled to distinct “clack” reporter moieties (e.g., cystine labels are coupled to blue reporter moieties, lysine labels are coupled to red reporter moieties, and tryptophan labels are coupled to green reporter moieties) in a single operation. A method may comprise coupling at least two “clack” reporter moieties or protecting groups to at least two “click” labels in a single operation. A method may comprise coupling at least three “clack” reporter moieties or protecting groups to at least three “click” labels in a single operation. A method may comprise coupling at least four “clack” reporter moieties or protecting groups to at least four “click” labels in a single operation. A method may comprise coupling at least five “clack” reporter moieties or protecting groups to at least five “click” labels in a single operation. The single operation may be an operation in which all “clack” reporter moieties are added simultaneously, react with labels simultaneously, or any combination thereof.

A method may comprise computationally designing a kit or set of reagents of the present disclosure. A user may input the type of method, the required sensitivity, the sample conditions (e.g., in vivo, ex vivo, or in vitro; pH, temperature, target concentration, etc.) into a program configured to optimize the set of reagents. The program may search a database of known proteomes, reactive species (e.g., glutathione concentration), and cytosolic conditions for a plurality of samples and organisms to generate an optimal reagent set. For example, the program may select a protease and a set of amino acid specific labels based on the amino acid abundances of a target organism.

In some cases, the kits may comprise a buffer. A buffer may refer to a species that can diminish the degree of pH change in a composition relative to the pH change that may occur if the buffer were not present. In some cases, a system of the present disclosure may comprise a buffer selected from the group consisting of sodium phosphate, HEPES, MES, and citrate. In some cases, a buffer may be in the form of a solid. In some cases, a buffer may be dissolved in a liquid. In some cases, a buffer may be adsorbed onto solid.

A solution of the present disclosure may comprise a buffer. The buffer may be present at 1 nM or higher concentrations. In some cases, the buffer may be present at 10 nM or higher concentrations. In some cases, the buffer may be present at 100 nM or higher concentrations. In some cases, the buffer is present at 1 μM or higher concentrations. The buffer may be present at 10 μM or higher concentrations. In some cases, the buffer may be present at 100 μM or higher concentrations. In some cases, the buffer may be present at 1 mM or higher concentrations. In some cases, the buffer may be present at 10 mM or higher concentrations. In some cases, the buffer may be present at 100 mM or higher concentrations. In some cases, the buffer may be present at 200 mM or higher concentrations. In some cases, the buffer may be present at 500 mM or higher concentrations.

In some cases, the solution comprising a buffer may have a pH of between 2 and 12. In some cases, the solution comprising a buffer may have a pH of between 2 and 6. In some cases, the solution comprising a buffer may have a pH of between 3 and 7. In some cases, the solution comprising a buffer may have a pH of between 4 and 8. In some cases, the solution comprising a buffer may have a pH of between 5 and 9. In some cases, the solution comprising a buffer may have a pH of between 6 and 10. In some cases, the solution comprising a buffer may have a pH of between 7 and 11. In some cases, the solution comprising a buffer may have a pH of between 8 and 12. In some cases, the solution comprising a buffer may have a pH of around 7.

### EXAMPLES

### Example 1: Capture of Native Protein, Non-Native Protein, Non-Native Polypeptide, and/or Non-Native Peptide on Support

A method for covalent and reversible binding of proteins to functionalized solid-support is performed as follows. A solid-support is modified with a polymer by grafting different PEGylated spacers, and then tested for protein capturing and releasing efficiency. The protein capture and release assay uses 10 μg standard proteins (consisting of bovine serum albumin and lysozyme). Protein yields from the capture and release assay are quantified for each functionalized solid-support using a bicinchoninic acid assay. To determine the lowest feasible concentration of standard proteins, the concentration of the standard proteins is varied from 1 μg to 10 μg in triplicate within the limits of detection of the bicinchoninic acid assay. The assay aims to determine lowest concentration of standard protein, which is detected with a bicinchoninic acid assay following capture and release with the solid-support.

The baseline assay uses, for example, 10 μg bovine serum albumin, pH 8 sodium phosphate for protein capture, and pH 4 citrate for protein release. Replicates of the assay are run with (1) altered PEGylated spacers and solid-support polymer matrices, and fixed amounts of bovine serum albumin (10 μg); (2) varying concentrations of bovine serum albumin and lysozyme (1 μg to 10 μg); (3) varying capture buffers; (4) varying release buffers (spanning a pH range of 4 to 6); and (5) varying temperature.

### Example 2: Native Protein, Non-Native Protein, Non-Native Polypeptide, and/or Non-Native Peptide Retention and Labeling

The structural integrity of the immobilized protein during the capturing and first labeling operation is tested using two channel Tandem mass tag (TMT)-based approach.

BSA is used as the standard protein in this experiment. BSA is immobilized on the support and then reacted with TMT (6)-126 dissolved in HEPES buffer to quantify the surface lysines. The labeled BSA is released from the support and the BSA is then denatured with 6M guanidine-HCl, which reduces and alkylates the cysteines on the denatured BSA. Following denaturation, the remaining lysines on the denatured BSA is labelled with TMT (6)-127. The labeled proteins are then digested with ArgC, and peptide level analysis is performed by nanoLC-MS/MS on Orbitrap Fusion. Additional experiments are performed that vary the capturing operations by: (a) addition of different salts (e.g., KCl, NaCl, MgCl₂) at (b) different concentrations (e.g., 1 millimolar (mM) and 100 mM) in pH 8 Na₃PO₄ buffer. 20/59 lysines are predicted to be solvent accessible through the prediction model generated by PISA-PDB. The lysines are roughly uniformly distributed between the solvent accessible regions and the buried regions. Some surface-exposed lysine is captured on the support, making these residues inaccessible for labeling

A positive and negative control are included in the experiment. The positive control is a BSA protein that is first labeled in solution phase with TMT (6)-126, precipitated in cold acetone, denatured with 6M guanidine-HCl, alkylated, and labeled with TMT (6)-127 in HEPES buffer. The negative control is a BSA protein that is first denatured, alkylated, and then labeled with TMT (6)-126 in HEPES buffer solution followed by quenching and TMT (6)-127 labeling.

For analysis, the extent of protein structure retention is determined by measuring the Cohen kappa value (e.g., concordant analysis) with predicted accessible surface area (ASA). This analysis determines which buffer conditions improves protein structure retention.

### Example 3: Discrimination Between Buried and Surface-Exposed Residues

BSA is incubated on a support modified with dialkyl maleic anhydride. The dialkyl maleic anhydride is attached to the surface via flexible PEG molecules. The surface-exposed amine groups (e.g., ε-amine on lysines or α-amine on the N-termini) covalently coupled with the dialkyl maleic anhydride on the support. Once the BSA is immobilized on the support, the surface lysine residues are blocked with NHS-propionate acetate, followed by labeling surface acidic residues through amine coupling (e.g., Click 1, amine azide) and EDC in pH 7.5 Na₃PO₄ buffer (e.g., pH between 7 to 8). The EDC functions as a coupling reagent. The surface tyrosines are labeled next through diazo coupling (e.g., Click 2, diazo-norborene) in pH 7.5 Na₃PO₄ buffer (e.g., pH between 7 to 8) as shown in Scheme 10. Acidic amino acid residues and tyrosine residues are chosen for labeling due to their relative equivalent expression on surface-exposed regions of proteins and buried regions of proteins, as shown in Table 3. Table 3 shows the computed weighted average surface accessibility (ASA) of the 20 amino acids across the PISA-PDB database; amino acids completely buried equals 0 and amino acids completely surface-accessible equals 1. A classification of amino acids to core, intermediate, or surface preponderance is denoted as class.

TABLE 3
Computed weighted average ASA.

| Amino acid | | ASA | Class |
|---|---|---|---|
| Cysterine | C | 0.268 | Core |
| Isoleucine | I | 0.273 | Core |
| Tryptophan | W | 0.279 | Core |
| Phenylalanine | F | 0.290 | Core |
| Valine | V | 0.306 | Core |
| Tyro | Y | 0.319 | Core |
| Leucine | L | 0.321 | Core |
| Methionine | M | 0.364 | Core |
| Alanine | A | 0.405 | Core |
| Histidine | H | 0.425 | Intermediate |
| Threonine | T | 0.480 | Intermediate |
| Proline | P | 0.502 | Intermediate |
| Arginine | R | 0.539 | Intermediate |
| Asparagine | N | 0.568 | Intermediate |
| Serine | S | 0.568 | Surface |
| Glutamine | Q | 0.573 | Surface |
| Glutamic Acid | E | 0.586 | Surface |
| Glycine | G | 0.588 | Surface |
| Lysine | K | 0.607 | Surface |
| Aspartic Acid | D | 0.615 | Surface |

The selected labeling molecules are small molecules that result in minimal disruption of the protein structure. Following labeling, the surface-exposed lysines are blocked to prevent cross-reactivity and the surface is washed to remove excess reagents.

Following washing, the labeled protein is released from the support in an acidic pH (e.g., pH<4) and denatured (e.g., denatured with guanidine-HCl). During the denaturing, the cysteine residues in the labeled protein are reduced and alkylated. The labeled protein is then digested by proteases (e.g., ArgC and ProAlanase) and the resulting peptides are captured on a pyridinecarboxaldehyde (PCA)-modified support.

Once recaptured, the internal acidic and tyrosine residues on the peptides are labeled; Acidic residues are labeled through amine coupling (e.g., Click 3, amine-aryl, aryliodide) and tyrosine residues are labeled through diazo coupling (e.g., Click 4, diazo-aldehyde), as shown in Scheme 11.

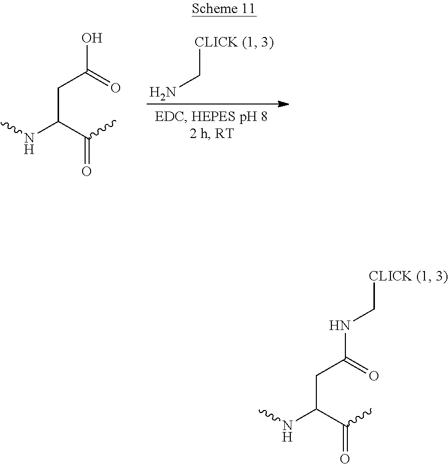

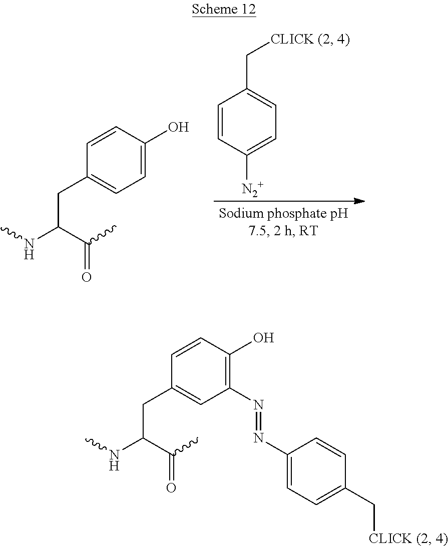

5 The partnering four fluorophores to the corresponding Click molecules are then added. Table 4 shows the first reactive groups (e.g., Click Partner) and second reactive groups (e.g., Clack Partner) for labeling the internal residues (e.g., Click 1 and Click 2) and the external residues (e.g., Click 3 and Click 4) and their corresponding fluorophores (e.g., Clack partner).

TABLE 4
First Reactive Groups and Second Reactive Groups for External and Internal
Amino Acid Labeling.

| Bioorthogonal | Click Partner | Clack Partner |
|---|---|---|
| chemistry | (labels amino-acids) | (Fluorophore conjugated) |
| Azide- DBCO CLICK 1 | 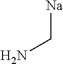 | 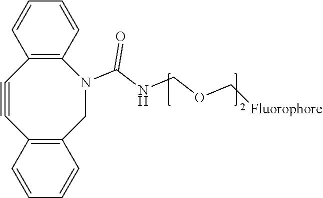 |
| Aryliodide- boronic acid CLICK 3 | 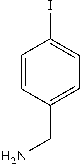 | 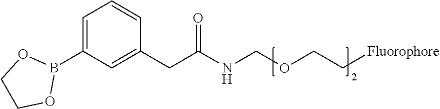 |
| aldehyde (protected)- dithiolane CLICK 2 | 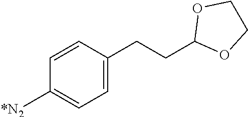 | 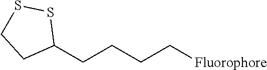 |
| norbornene- tetrazine CLICK 4 | 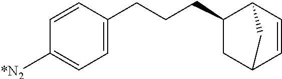 | 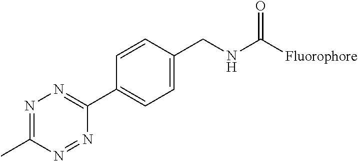 |

Additionally, C-terminal differentiation is performed using a photocatalyzed C-terminal decarboxylative alkylation using a Michael acceptor, such as, for example, as described in Zhang, L., et al. “Photoredox-catalyzed decarboxylative C-terminal differentiation for bulk and single molecule proteomics.” ACS Chemical Biology, 2021, 16, 11, 2595-2603, which is hereby incorporated by reference. The labeled peptides are released from the PCA-modified surface using dimethylaminoethylhydrazine.

Once the internal residues are labeled, the peptides are analyzed with fluorosequencing and the pattern of surface-exposed acidic and tyrosine residues and the pattern of buried acidic and tyrosine residues.

The experiment is performed in triplicate and under two conditions: (1) incubating the BSA in PBS prior to the capture of the BSA on the dialkyl maleic anhydride-modified surface and (2) heat-denaturing the BSA (e.g., incubating BSA at 95° C. for 10 minutes).

Analysis of the fluorosequencing data generates individual profiles of acidic residues and tyrosine residues labeled as exposed (1) or buried (0). The individual profiles are compared to reference protein sequences (e.g., RNA translated sequences). The comparison data is then combined with data from the different proteases used in the digesting operation. By mapping to the reference sequence and then incorporating the protease data, the binary heatmap is produced that shows a binary observation of exposed/buried residues for individual observations. Clustering analysis is then used to indicate the heterogeneity of the protein structures.

Additionally, validating measures with other reference studies or tools (e.g., PISA-PDB) calculates intraclass correlation and concordance correlation coefficient (e.g., Cohen's kappa, k) statistical values. The correlation values measure lysines identified as the solvent accessible class (e.g., through reference datasets) with continuous lysine frequency tagged as labeled susceptible class (e.g., through the solid-phase labeling chemistry experiments. A measure of k>0.8 is considered as positively concordant, and a measure of k<0.4 is considered as negatively concordant.

ASA prediction models of BSA (e.g., PISA-PDB) of the labeled amino acids predicts that there are 59 glutamic acid residues (20 inaccessible), 39 aspartic acid residues (13 inaccessible), and 20 tyrosine residues (10 inaccessible). The results from the fluorosequencing analysis are compared with the existing BSA structure and the ASA prediction tool for concordance.

### Example 4: Enzymatic Activity and Structural Retention Following Solid-Support Capture and Release

The retention of enzymatic and 2D structures of the captured/released proteins can be demonstrated as follows. As mild conditions for capturing (pH 8) and releasing (pH 4) of intact proteins can have minimal impact on protein structure and enzymatic activity, circular dichroism (CD) may be used for structural determination of bovine serum albumin before capturing and after releasing from the solid-support. In parallel, the enzymatic activity of lysozymes can be measured before capturing and after releasing from the solid-support. In each assay, similar results before capturing and after releasing can demonstrate the structural integrity of proteins through the process.

To alter solvation of a support, the support surface may be modified with a polymer by grafting different types of PEGylated spacers (e.g., PEG4 and PEG16), and the capturing and releasing efficiency of proteins (e.g., 10 μg) may be analyzed on these functionalized supports. An assay for determining protein and/or peptide capture and release efficiency by a solid-support may comprise: (1) solubilizing 20 μg standard proteins (e.g., BSA and lysozyme) in 200 μL of 100 mM pH 8 sodium phosphate buffer, and quantifying protein capture and release yields on the variety of functionalized solid supports, using the concentrations as calibration standards (100 μg/mL). (2) Next, the standard proteins may be incubated with the different functionalized lanterns for 1, 2, 4 and 8 hours. (3) Following the incubation, the flow-through solution may be collected, and uncaptured proteins may be quantified. (4) A series of wash operations (e.g., with pH 7 sodium phosphate buffer) may be performed, and proteins may be released from the supports (e.g., by incubating a dimethylmaleimide functionalized solid-support in 200 μL of pH 4 citrate buffer). The protein released from the solid-support may be quantified.

The pH of the release buffer can be varied to determine the optimal conditions to reasonably maintain protein structure. Enzymatic activity assay for lysozyme can include multiple capture and release buffers. The ‘before capture’ and ‘after release’ samples can be diluted to ensure similar conditions across replicates. Lysozyme enzymatic activity can be measured by monitoring the disappearance of a 450 nm absorbance band, using Micrococcus lysodeikticus cells as a substrate. Bovine serum albumin structural integrity before capture and after release can be measured with circular dichroism.

An example of an assay may comprise: (1) establishing conditions for capturing and maintenance of protein fold; (2) incubating 20 μg of BSA with a dialkyl maleic anhydride-functionalized lantern (e.g., the lantern may comprise a protein and/or peptide loading capacity of 4 μmol); (3) blocking surface lysine residues with NHS-propionate acetate; (4) labeling acidic residues with 200 μmole of an amine-azide (5) functionalizing the amine-azide with 400 μmol of EDC in 200 μL pH 8 sodium bicarbonate buffer; (6) labeling tyrosines with a micromolar quantity of a diazo-norbornene in pH 7.5 sodium phosphate buffer; (7) following the labeling of surface exposed side chains of acidic residues and tyrosine, releasing the proteins from the solid-support with an acidic pH; (8) denaturing the proteins with guanidine-HCl; (9) reducing and alkylating cysteine residues; (10) digesting with argC and proalanase; (11) capturing peptides pyridine carboxaldehyde-functionalized lanterns; (12) labeling internal acidic residues with amine-aryl iodide; (13) labeling internal tyrosine residues with a diazo-aldehyde; (14) fluorosequencing and analyzing the pattern of labels to determine whether acidic residues and tyrosine residues surface-exposed in the intact protein; (15) repeating the assay in triplicate under two conditions prior to the fluorosequencing process, comprising (a) incubating BSA in PBS, and (b) heat-denaturation by heating the protein 95° C. for 10 minutes. The labeling scheme and reagents outlined in this example are shown in FIG. 5.

The analysis from fluorosequencing may produce different profiles for each type of sample treatment. Through crystal structure analysis, BSA may comprise 59 Glutamate residues, of which 20 may be buried; 39 aspartate residues, of which 13 may be buried; and 20 tyrosine residues, of which 10 may be buried. As BSA may comprise 23 arginine residues, 47 alanine residues, and 20 proline residues, digestion with both argC and proalanase protease may generate peptide fragments of different and complementary lengths.

### Example 5: Discriminating Buried and Surface Regions of Bovine Serum Albumin

Buried regions and surface regions of bovine serum albumin can be distinguished with a fluorosequencing assay. Bovine serum albumin can be captured on a solid-support, and its solvent exposed lysines and acidic residues can be coupled to a first set of labels. The bovine serum albumin can then be released from the solid support, and unlabeled lysines and acidic residues (not coupled to the first set of labels) of the peptide fragments can be coupled to a second set of labels. The bovine serum albumin can then be digested with clostripain (ArgC) to yield peptide fragments. Positions of the first set of labels and second set of labels in the peptide fragments can be determined with fluorosequencing, such that different regions of the bovine serum albumin can be profiled. Residues which are identified as belonging to buried and surface regions can be compared to existing structural models of bovine serum albumin.

The assay can comprise: (1) labeling the bovine serum albumin surface lysines and acidic residues while the bovine serum albumin is coupled to a solid-support; (2) release the bovine serum albumin; (3) denature the bovine serum; (4) label buried lysines and acidic residues; (5) perform clostripain (ArgC) digestion on the labeled bovine serum albumin. As a control, the frequency of lysine, aspartate and glutamate, arginine, and tyrosine can also be measured and compared to bovine serum albumin's known composition.

To test the structural integrity of proteins during capturing and labeling operations, a two channel Tandem mass tag (TMT)-based approach and tandem mass-spectrometry may be used as an orthogonal test. For example, a specific assay may comprise: Immobilize (20 μg) BSA on a lantern, react 0.8 mg of a first tandem mass tag dissolved in 200 μL HEPES buffer to quantify the susceptible or surface lysines. Following the release, denature the protein with 6M guanidine-HCl, reduce and alkylate the cysteines, and then label all remaining amines with a second tandem mass tag. As a positive control: first label BSA in solution phase with the first tandem mass tag, precipitate the proteins in cold acetone, denature the proteins with 6M guanidine-HCl, alkylate, and then label with the second tandem mass tag in 0.1M HEPES buffer. As a negative control: denature BSA, alkylate and label with the first tandem mass tag in HEPES buffer solution, quench, and then label with the second tandem mass tag. As a screen of buffer conditions to maintain BSA protein structure, the capturing operations may be varied by—(a) adding different salts, such as KCl, NaCl, MgCl₂, (b) vary protein concentrations (e.g., 1 mM to 100 mM) in sodium phosphate buffer (pH 8). The tandem mass tag labeled protein samples may then be digested (e.g., with ArgC) and peptide level analysis may be performed by tandem mass spectrometry. Results may be compared with a predicted structural model (e.g., a model generated by PISA-PDB).

From analysis of a crystal structure of BSA, 20 of 59 lysines are predicted to be solvent accessible. With such a large number of available lysine residues to characterize and roughly uniform distribution of lysines between solvent accessible/buried regions, the analysis proposed above may in part represent the extent to which the crystal structure conformation was retained when BSA is coupled to solid support. It may be noted that some of the surface exposed lysines may be covalently captured by the dialkyl maleic anhydride group on lanterns, which may render them inert towards tandem mass tag labels. However, which lysine(s) of the BSA bind to the solid-support may be random, and therefore solid-support blocked lysines may be averaged.

The above assay may assess the extent of protein structure retention by identifying or determining the Cohen kappa value (concordant analysis) with predicted ASA. A measure of kappa-cohen may be (k)>0.8 through the process. The pH of the release buffer may be varied, and (b) the buffer conditions which promote protein structural retention may be identified. As a control, the labeling and digestion process is verified with mass spectrometry. In this control assay, a small dummy label can be coupled to surface region lysine residues, and different labels can be coupled to buried lysines. The bovine serum albumin can then be digested with clostripain (ArgC) and analyzed with tandem mass spectrometry.

Additional control assays may include:
Perform fluorophore labeling; fluorosequencing between the bovine serum albumin exposed and buried residues.
Look for concordance between fluorosequencing of the amino acids and the composition and sequence of bovine serum albumin listed in the Protein Data Bank.
The assay determines greater than 75% concordance with existing bovine serum albumin structure and workflow.

### Example 6: Discriminate Structure of Related Isoforms and Oligomeric Forms of Proteins

The recombinant forms of full-length (758AA) and truncated (441 AA) forms of Tau protein can be analyzed by the workflow outlined in Example 3, and the results can be used to discriminate the two isoforms. Similarly, monomeric and oligomeric forms of alpha-synuclein may be discriminated using the same workflow. The assay can include triplicate measurements of the Tau protein isoforms, triplicate measurements of the alpha-synuclein oligomers and monomers, as well as statistical analyses to discriminate between the different forms of Tau protein and alpha-synuclein. The assay is used to generate a computational algorithm to describe the expected model (alpha-fold) and the labeling scheme to maximize answer. 1. Pull-down purification can be performed on multiple proteins, including Tau protein and alpha-synuclein from biological sources. Perform labeling and characterization. The assay can also be automated. The assay can establish the discriminatory power to distinguish the different proteoforms of Tau protein and alpha-synuclein. Test proteins may be selected to establish a method for profiling physiological and disease associated variants of proteins. Apart from differences in folded protein structures caused through underlying mutated transcripts, the effect of aggregation and masking of the interface residues is often unclear.

### EMBODIMENTS

The following are illustrative examples of embodiments of the present disclosure and are not meant to be limiting in any way.

1. A method, comprising: providing a native protein coupled to a support, wherein the native protein comprises a native structure, wherein the native protein comprises one or more internal amino acids and one or more external amino acids, wherein the native protein comprises one or more labels coupled to the one or more external amino acids.

2. The method of embodiment 1, further comprising detecting the one or more labels coupled to the one or more external amino acids to determine the native structure of said native protein using the detected one or more labels.

3. The method of embodiment 2, wherein the detecting comprises detecting a sequence pattern of the one or more labels coupled to the one or more external amino acids.

4. The method of embodiment 2, wherein determining the native structure of the native protein comprises identifying a misfolded protein.

5. The method of any one of embodiments 1-5, wherein the one or more external amino acids comprises a surface-exposed amino acids residue.

6. The method of any one of embodiments 1-5, further comprising coupling the one or more labels to the one or more external amino acids.

7. The method of embodiment 6, further comprising coupling the native protein to the support prior to coupling the one or more labels to the one or more external amino acids.

8. The method of any one of embodiments 1-7, further comprising detecting the one or more labels coupled to the one or more external amino acids to quantify the native protein.

9. The method of any one of embodiments 1-8, further comprising releasing the native protein from the support.

10. The method of embodiment 9, further comprising digesting the native protein into one or more peptides.

11. The method of embodiment 10, wherein each of the one or more peptides has from about 5 amino acids to about 50 amino acids.

12. The method of embodiment 9, further comprising coupling one or more additional labels to the one or more internal amino acids.

13. The method of embodiment 12, further comprising detecting the one or more labels coupled to the one or more external amino acids.

14. The method of embodiment 9, further comprising coupling the one or more labels to the one or more external amino acids.

15. The method of embodiment 14, further comprising detecting one or more additional labels coupled to the one or more internal amino acids.

16. The method of any one of embodiments 1-15, further comprising using the one or more labels and one or more additional labels to determine the native structure of the native protein.

17. The method of any one of embodiments 1-16, wherein the native structure of the native protein comprises a tertiary structure of the native protein.

18. The method of any one of embodiments 1-17, wherein the one or more labels comprise an optical label.

19. The method of embodiment 18, wherein the optical label comprises a fluorescent dye.

20. The method of any one of embodiments 1-19, further comprising detecting the native structure of the native protein while the native protein is coupled to the support.

21. The method of any one of embodiments 1-20, wherein the native protein comprising the native structure is immobilized to the support.

22. The method of any one of embodiments 1-21, wherein the native protein is covalently-coupled to the support.

23. The method of any one of embodiments 1-22, wherein the native protein is reversibly-coupled to the support.

24. The method of any one of embodiments 1-23, wherein a surface of the support comprises one or more maleic anhydride groups.

25. The method of any one of embodiments 1-24, wherein the one or more labels comprise an amino acid type-specific label.

26. The method of embodiment 25, wherein the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

27. A system, comprising: a native protein coupled to a support, wherein the native protein comprises a native structure, wherein the native protein comprises one or more internal amino acids and one or more external amino acids, wherein the native protein comprises one or more labels coupled to the one or more external amino acids.

28. The system of embodiment 27, wherein the native structure comprises a tertiary structure of the native protein.

29. The system of embodiment 27 or embodiment 28, wherein the one or more labels are covalently-coupled to the one or more external amino acids.

30. The system of any one of embodiments 27-29, wherein the one or more labels comprise an optical label.

31. The system of embodiment 30, wherein the optical label comprises a fluorescent dye.

32. The system of any one of embodiments 27-31, wherein the native protein is covalently-coupled to the support.

33. The system of any one of embodiments 27-32, wherein the native protein is reversibly-coupled to the support.

34. The system of any one of embodiments 27-33, wherein a surface of the support comprises one or more maleic anhydride groups.

35. The system of any one of embodiments 27-34, wherein the one or more labels comprise an amino acid type-specific label.

36. The system of embodiment 35, wherein the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

37. The system of any one of embodiments 27-36, wherein the one or more external amino acids comprises a surface-exposed amino acid residue.

38. The system of any one of embodiments 27-37, further comprising a detector configured to detect the one or more labels.

39. The system of embodiment 38, wherein the detector comprises an intensified charge-couple device (CCD) detector or a complementary metal-oxide semiconductor (CMOS) detector.

40. The system of embodiment 38, further comprising a computer processor communicatively coupled to the detector, wherein the computer processor is programmed to detect one or more signals from the detector.

41. The system of embodiment 40, wherein the one or more signals are from the one or more labels coupled to the one or more external amino acids.

42. The system of embodiment 40, wherein the computer processor is programmed to determine the native structure of the native protein using the one or more labels detected by the detector.

43. The system of embodiment 40, wherein the computer processor is programmed to detect fluorescence signals.

44. The system of embodiment 40, wherein the computer processor is programmed to distinguish each of the one or more signals from the detector.

45. The system of embodiment 40, wherein the computer processor is programmed to quantify the one or more signals from the detector.

46. A method, comprising: providing a native protein, wherein the native protein comprises a native structure, wherein the native protein comprises one or more internal amino acids and one or more external amino acids, wherein the native protein comprises one or more optically detectable labels coupled to the one or more external amino acids.

47. The method of embodiment 46, further comprising detecting the one or more optically detectable labels coupled to the one or more external amino acids to identify the native structure of the native protein using the detected one or more optically detectable labels.

48. The method of embodiment 47, wherein the detecting comprises detecting a sequence pattern of the one or more labels coupled to the one or more external amino acids.

49. The method of embodiment 47, wherein determining the native structure of the native protein comprises identifying a misfolded protein.

50. The method of any one embodiments 46-49, wherein the one or more external amino acids comprises a surface-exposed amino acid residue.

51. The method of any one of embodiments 46-50, further comprising coupling the one or more labels to the one or more external amino acids.

52. The method of embodiment 51, further comprising coupling the native protein to the support prior to coupling the one or more labels to the one or more external amino acids.

53. The method of any one of embodiments 46-52, further comprising detecting the one or more labels coupled to the one or more external amino acids to quantify the native protein.

54. The method of any one of embodiments 46-53, further comprising releasing the native protein from the support.

55. The method of embodiment 54, further comprising digesting the native protein into one or more peptides.

56. The method of embodiment 55, wherein each of the one or more peptides has from about 5 amino acids to about 50 amino acids.

57. The method of embodiment 54, further comprising coupling one or more additional labels to the one or more internal amino acids.

58. The method of embodiment 57, further comprising detecting the one or more labels coupled to the one or more external amino acids.

59. The method of embodiment 54, further comprising coupling the one or more labels to the one or more external amino acids.

60. The method of embodiment 59, further comprising detecting one or more additional labels coupled to the one or more internal amino acids.

61. The method of any one of embodiments 46-60, further comprising using the one or more labels and one or more additional labels to determine the native structure of the native protein.

62. The method of any one of embodiments 46-61, wherein the native structure of the native protein comprises a tertiary structure of the native protein.

63. The method of any one of embodiments 46-62, wherein the one or more labels comprise an optical label.

64. The method of embodiment 63, wherein the optical label comprises a fluorescent dye.

65. The method of any one of embodiments 46-64, further comprising detecting the native structure of the native protein while the native protein is coupled to the support.

66. The method of any one of embodiments 46-65, wherein the native protein comprising the native structure is immobilized to the support.

67. The method of any one of embodiments 46-66, wherein the native protein is covalently-coupled to the support.

68. The method of any one of embodiments 46-67, wherein the native protein is reversibly-coupled to the support.

69. The method of any one of embodiments 46-68, wherein a surface of the support comprises one or more maleic anhydride groups.

70. The method of any one of embodiments 46-69, wherein the one or more labels comprise an amino acid type-specific label.

71. The method of embodiment 70, wherein the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

72. A system, comprising: a native protein, wherein the native protein comprises a native structure, wherein the native protein comprises one or more internal amino acids and one or more external amino acids, wherein the native protein comprises one or more optically detectable labels coupled to one or more external amino acids.

73. The system of embodiment 72, wherein the native structure comprises a tertiary structure of the native protein.

74. The system of embodiment 72 or embodiment 73, wherein the one or more labels are covalently-coupled to the one or more external amino acids.

75. The system of any one of embodiments 72-74, wherein the one or more labels comprise an optical label.

76. The system of embodiment 75, wherein the optical label comprises a fluorescent dye.

77. The system of any one of embodiments 72-76, wherein the native protein is covalently-coupled to the support.

78. The system of any one of embodiments 72-77, wherein the native protein is reversibly-coupled to the support.

79. The system of any one of embodiments 72-78, wherein a surface of the support comprises one or more maleic anhydride groups.

80. The system of any one of embodiments 72-79, wherein the one or more labels comprise an amino acid type-specific label.

81. The system of embodiment 80, wherein the amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

82. The system of any one of embodiments 72-81, wherein the one or more external amino acids comprises a surface-exposed amino acid residue.

83. The system of any one of embodiments 72-82, further comprising a detector configured to detect the one or more labels.

84. The system of embodiment 83, wherein the detector comprises an intensified charge-couple device (CCD) detector or a complementary metal-oxide semiconductor (CMOS) detector.

85. The system of embodiment 83, further comprising a computer processor communicatively coupled to the detector, wherein the computer processor is programmed to detect one or more signals from the detector.

86. The system of embodiment 85, wherein the one or more signals are from the one or more optically detectable labels coupled to the one or more external amino acids.

87. The system of embodiment 85, wherein the computer processor is programmed to determine the native structure of the native protein using the one or more optically detectable labels detected by the detector.

88. The system of embodiment 85, wherein the computer processor is programmed to detect fluorescence signals.

89. The system of embodiment 85, wherein the computer processor is programmed to distinguish each of the one or more signals from the detector.

90. The system of system 85, wherein the computer processor is programmed to quantify the one or more signals from the detector.

91. A method of determining the native structure of a protein, comprising:
a) providing the protein in its native structure coupled to a solid surface;
b) subsequent to a), coupling one or more labels to the protein;
c) releasing the protein from said solid surface;
d) digesting the protein to form one or more peptides;
e) coupling one or more additional labels to the one or more peptides;
f) detecting signals or signal changes from the one or more labels and the one or more additional labels; and
g) using the signals or signal changes to determine the native structure of the protein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method, comprising:
providing a native protein coupled to a support, wherein said native protein comprises a native structure, wherein said native protein comprises one or more internal amino acids and one or more external amino acids, wherein said native protein comprises one or more labels coupled to said one or more external amino acids.

2. The method of claim 1, further comprising detecting said one or more labels coupled to said one or more external amino acids to determine said native structure of said native protein using said detected one or more labels.

3. The method of claim 2, wherein said detecting comprises detecting a sequence pattern of said one or more labels coupled to said one or more external amino acids.

4. The method of claim 2, wherein determining said native structure of said native protein comprises identifying a misfolded protein.

5. The method of claim 1, wherein said one or more external amino acids comprises a surface-exposed amino acid residue.

6. The method of claim 1, further comprising coupling said one or more labels to said one or more external amino acids.

7. The method of claim 6, further comprising coupling said native protein to said support prior to coupling said one or more labels to said one or more external amino acids.

8. The method of claim 1, further comprising detecting said one or more labels coupled to said one or more external amino acids to quantify said native protein.

9. The method of claim 1, further comprising releasing said native protein from said support.

10. The method of claim 9, further comprising digesting said native protein into one or more peptides.

11. The method of claim 10, wherein each of said one or more peptides has from about 5 amino acids to about 50 amino acids.

12. The method of claim 9, further comprising coupling one or more additional labels to said one or more internal amino acids.

13. The method of claim 12, further comprising detecting said one or more labels coupled to said one or more external amino acids.

14. The method of claim 9, further comprising coupling said one or more labels to said one or more external amino acids.

15. The method of claim 14, further comprising detecting one or more additional labels coupled to said one or more internal amino acids.

16. The method of claim 1, further comprising using said one or more labels and one or more additional labels to determine said native structure of said native protein.

17. The method of claim 1, wherein said native structure of said native protein comprises a tertiary structure of said native protein.

18. The method of claim 1, wherein said one or more labels comprise an optical label.

19. The method of claim 18, wherein said optical label comprises a fluorescent dye.

20. The method of claim 1, further comprising detecting said native structure of said native protein while said native protein is coupled to said support.

21. The method of claim 1, wherein said native protein comprising said native structure is immobilized to said support.

22. The method of claim 1, wherein said native protein is covalently-coupled to said support.

23. The method of claim 1, wherein said native protein is reversibly-coupled to said support.

24. The method of claim 1, wherein a surface of said support comprises one or more maleic anhydride groups.

25. The method of claim 1, wherein said one or more labels comprise an amino acid type-specific label.

26. The method of claim 25, wherein said amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

27. A system, comprising: a native protein coupled to a support, wherein said native protein comprises a native structure, wherein said native protein comprises one or more internal amino acids and one or more external amino acids, wherein said native protein comprises one or more labels coupled to said one or more external amino acids.

28. The system of claim 27, wherein said native structure comprises a tertiary structure of said native protein.

29. The system of claim 27, wherein said one or more labels are covalently-coupled to said one or more external amino acids.

30. The system of claim 27, wherein said one or more labels comprise an optical label.

31. The system of claim 30, wherein said optical label comprises a fluorescent dye.

32. The system of claim 27, wherein said native protein is covalently-coupled to said support.

33. The system of claim 27, wherein said native protein is reversibly-coupled to said support.

34. The system of claim 27, wherein a surface of said support comprises one or more maleic anhydride groups.

35. The system of claim 27, wherein said one or more labels comprise an amino acid type-specific label.

36. The system of claim 35, wherein said amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

37. The system of claim 27, wherein said one or more external amino acids comprises a surface-exposed amino acid residue.

38. The system of claim 27, further comprising a detector configured to detect said one or more labels.

39. The system of claim 38, wherein said detector comprises an intensified charge-couple device (CCD) detector or a complementary metal-oxide semiconductor (CMOS) detector.

40. The system of claim 38, further comprising a computer processor communicatively coupled to said detector, wherein said computer processor is programmed to detect one or more signals from said detector.

41. The system of claim 40, wherein said one or more signals are from said one or more labels coupled to said one or more external amino acids.

42. The system of claim 40, wherein said computer processor is programmed to determine said native structure of said native protein using said one or more labels detected by said detector.

43. The system of claim 40, wherein said computer processor is programmed to detect fluorescence signals.

44. The system of claim 40, wherein said computer processor is programmed to distinguish each of said one or more signals from said detector.

45. The system of claim 40, wherein said computer processor is programmed to quantify said one or more signals from said detector.

46. A method, comprising:
providing a native protein, wherein said native protein comprises a native structure, wherein said native protein comprises one or more internal amino acids and one or more external amino acids, wherein said native protein comprises one or more optically detectable labels coupled to said one or more external amino acids.

47. The method of claim 46, further comprising detecting said one or more optically detectable labels coupled to said one or more external amino acids to identify said native structure of said native protein using said detected one or more optically detectable labels.

48. The method of claim 47, wherein said detecting comprises detecting a sequence pattern of said one or more labels coupled to said one or more external amino acids.

49. The method of claim 47, wherein determining said native structure of said native protein comprises identifying a misfolded protein.

50. The method of claim 46, wherein said one or more external amino acids comprises a surface-exposed amino acid residue.

51. The method of claim 46, further comprising coupling said one or more labels to said one or more external amino acids.

52. The method of claim 51, further comprising coupling said native protein to said support prior to coupling said one or more labels to said one or more external amino acids.

53. The method of claim 46, further comprising detecting said one or more labels coupled to said one or more external amino acids to quantify said native protein.

54. The method of claim 46, further comprising releasing said native protein from said support.

55. The method of claim 54, further comprising digesting said native protein into one or more peptides.

56. The method of claim 55, wherein each of said one or more peptides has from about 5 amino acids to about 50 amino acids.

57. The method of claim 54, further comprising coupling one or more additional labels to said one or more internal amino acids.

58. The method of claim 57, further comprising detecting said one or more labels coupled to said one or more external amino acids.

59. The method of claim 54, further comprising coupling said one or more labels to said one or more external amino acids.

60. The method of claim 59, further comprising detecting one or more additional labels coupled to said one or more internal amino acids.

61. The method of claim 46, further comprising using said one or more labels and one or more additional labels to determine said native structure of said native protein.

62. The method of claim 46, wherein said native structure of said native protein comprises a tertiary structure of said native protein.

63. The method of claim 46, wherein said one or more labels comprise an optical label.

64. The method of claim 63, wherein said optical label comprises a fluorescent dye.

65. The method of claim 46, further comprising detecting said native structure of said native protein while said native protein is coupled to said support.

66. The method of claim 46, wherein said native protein comprising said native structure is immobilized to said support.

67. The method of claim 46, wherein said native protein is covalently-coupled to said support.

68. The method of claim 46, wherein said native protein is reversibly-coupled to said support.

69. The method of claim 46, wherein a surface of said support comprises one or more maleic anhydride groups.

70. The method of claim 46, wherein said one or more labels comprise an amino acid type-specific label.

71. The method of claim 70, wherein said amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

72. A system, comprising: a native protein, wherein said native protein comprises a native structure, wherein said native protein comprises one or more internal amino acids and one or more external amino acids, wherein said native protein comprises one or more optically detectable labels coupled to one or more external amino acids.

73. The system of claim 72, wherein said native structure comprises a tertiary structure of said native protein.

74. The system of claim 72, wherein said one or more labels are covalently-coupled to said one or more external amino acids.

75. The system of claim 72, wherein said one or more labels comprise an optical label.

76. The system of claim 75, wherein said optical label comprises a fluorescent dye.

77. The system of claim 72, wherein said native protein is covalently-coupled to said support.

78. The system of claim 72, wherein said native protein is reversibly-coupled to said support.

79. The system of claim 72, wherein a surface of said support comprises one or more maleic anhydride groups.

80. The system of claim 72, wherein said one or more labels comprise an amino acid type-specific label.

81. The system of claim 80, wherein said amino acid type-specific label comprises a lysine-specific label, a cysteine-specific label, a tyrosine-specific label, a tryptophan-specific label, a histidine-specific label, a serine-specific label, a threonine-specific label, an arginine-specific label, a glutamic acid-specific label, an aspartic acid-specific label, or any combination thereof.

82. The system of claim 72, wherein said one or more external amino acids comprises a surface-exposed amino acid residue.

83. The system of claim 72, further comprising a detector configured to detect said one or more optically detectable labels.

84. The system of claim 83, wherein said detector comprises an intensified charge-couple device (CCD) detector or a complementary metal-oxide semiconductor (CMOS) detector.

85. The system of claim 83, further comprising a computer processor communicatively coupled to said detector, wherein said computer processor is programmed to detect one or more signals from said detector.

86. The system of claim 85, wherein said one or more signals are from said one or more optically detectable labels coupled to said one or more external amino acids.

87. The system of claim 85, wherein said computer processor is programmed to determine said native structure of said native protein using said one or more optically detectable labels detected by said detector.

88. The system of claim 85, wherein said computer processor is programmed to detect fluorescence signals.

89. The system of claim 85, wherein said computer processor is programmed to distinguish each of said one or more signals from said detector.

90. The system of claim 85, wherein said computer processor is programmed to quantify said one or more signals from said detector.
